SEQUENCE LISTING (62 sequences)

SEQ ID NO: 1 - DNA, 21 bp, Artificial Sequence
  shRNA against TGFBR2
ggagaaagaatgacgagaaca

SEQ ID NO: 2 - DNA, 21 bp, Artificial Sequence
  shRNA against TGFBR2
gctccagaagtcctagaatcc

SEQ ID NO: 3 - DNA, 21 bp, Artificial Sequence
  shRNA against TGFBR2
ggaggagaagattcctgaaga

SEQ ID NO: 4 - DNA, 21 bp, Artificial Sequence
  shRNA against TGFBR2
gcatagaggcgcctagaaatt

SEQ ID NO: 5 - DNA, 21 bp, Artificial Sequence
  shRNA against TGFBR2
ggttcctgtgtgcccttattt

SEQ ID NO: 6 - DNA, 21 bp, Artificial Sequence
  shRNA against TGFBR2
gcgcctagaaattccacttgc

SEQ ID NO: 7 - DNA, 19 bp, Artificial Sequence
  shRNA against TGFBR2
tcctgcatgagcaactgca

SEQ ID NO: 8 - DNA, 19 bp, Artificial Sequence
  shRNA against TGFBR2
gcttctccaaagtgcatta

SEQ ID NO: 9 - DNA, 19 bp, Artificial Sequence
  shRNA against TGFBR2
ccacgtgtgccaacaacat

SEQ ID NO: 10 - DNA, 19 bp, Artificial Sequence
  shRNA against TGFBR2
gctccaatatcctcgtgaa

SEQ ID NO: 11 - DNA, 19 bp, Artificial Sequence
  shRNA against TGFBR2
cctgtgtcgaaagcatgaa

SEQ ID NO: 12 - DNA, 19 bp, Artificial Sequence
  shRNA against TGFBR2
gctccctaaacactaccaa

SEQ ID NO: 13 - DNA, 21 bp, Artificial Sequence
  shRNA against PD1
tggaacccattcctgaaatta

SEQ ID NO: 14 - DNA, 21 bp, Artificial Sequence
  shRNA against PD1
ggaacccattcctgaaattat

SEQ ID NO: 15 - DNA, 21 bp, Artificial Sequence
  shRNA against PD1
gaacccattcctgaaattatt

SEQ ID NO: 16 - DNA, 21 bp, Artificial Sequence
  shRNA against PD1
acccattcctgaaattattta

SEQ ID NO: 17 - DNA, 21 bp, Artificial Sequence
  shRNA against PD1
cccattcctgaaattatttaa

SEQ ID NO: 18 - DNA, 21 bp, Artificial Sequence
  shRNA against PD1
ccttccctgtggttctattat

SEQ ID NO: 19 - DNA, 21 bp, Artificial Sequence
  shRNA against PD1
cttccctgtggttctattata

SEQ ID NO: 20 - DNA, 21 bp, Artificial Sequence
  shRNA against PD1
ttccctgtggttctattatat

SEQ ID NO: 21 - DNA, 21 bp, Artificial Sequence
  shRNA against PD1
tccctgtggttctattatatt

SEQ ID NO: 22 - DNA, 21 bp, Artificial Sequence
  shRNA against PD1
ccctgtggttctattatatta

SEQ ID NO: 23 - DNA, 21 bp, Artificial Sequence
  shRNA against PD1
cctgtggttctattatattat

SEQ ID NO: 24 - DNA, 19 bp, Artificial Sequence
  TGFBR2 primer Forward
acgtgttgagagatcgagg

SEQ ID NO: 25 - DNA, 20 bp, Artificial Sequence
  TGFBR2 primer Reverse
cccagcactcagtcaacgtc

SEQ ID NO: 26 - DNA, 20 bp, Artificial Sequence
  PD-1 primer Forward
cctccacctttacacatgcc

SEQ ID NO: 27 - DNA, 20 bp, Artificial Sequence
  PD-1 primer Reverse
cttactgcctcagcttccct

SEQ ID NO: 28 - DNA, 47 bp, Artificial Sequence
  Cloning primers
ggtatactctagacatatggctagcactagtcaaaaacctgtggttc

SEQ ID NO: 29 - DNA, 26 bp, Artificial Sequence
  Cloning primers
ttgtaccgttaacgatccgacgccgc

SEQ ID NO: 30 - DNA, 102 bp, Artificial Sequence
  Cloning primers
tgactagtcaaaaacctgtggttctattatattattctcttgaaataatataatagaaccacaggcggtgtttcgtcctttccacaagatatataaagccaa

SEQ ID NO: 31 - DNA, 50 bp, Artificial Sequence
  Cloning primers
gtaccgttaacaaggtcgggcaggaagagggcctatttcccatgattcct

SEQ ID NO: 32 - DNA, 39 bp, Artificial Sequence
  Cloning primers
aggactagtcaaaaaggaattcgctcagaagaaatctct

SEQ ID NO: 33 - DNA, 27 bp, Artificial Sequence
  Cloning primers
ctagctagcgatccgacgccgccatct

SEQ ID NO: 34 - DNA, 42 bp, Artificial Sequence
  Cloning primers
atgttaaccaaaaacctgtggttctattatattattctcttg

SEQ ID NO: 35 - DNA, 34 bp, Artificial Sequence
  Cloning primers
tcactagtaaggtcgggcaggaagagggcctatt

SEQ ID NO: 36 - DNA, 29 bp, Artificial Sequence
  Cloning primers
taggccctcactagtgatccgacgccgcc

SEQ ID NO: 37 - DNA, 38 bp, Artificial Sequence
  Cloning primers
ctagctagccaaaaaggaattcgctcagaagaaatctc

SEQ ID NO: 38 - DNA, 409 bp, Artificial Sequence
  shPD-1-mU6-MCS base sequence
gttaacgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcctgtggttctattatattatttcaagagaataatataatagaaccacaggtttttgactagtgctagccatatgtctagagtatac

SEQ ID NO: 39 - DNA, 328 bp, Artificial Sequence
  hU6-shPD-1 base sequence
gttaacaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcctgtggttctattatattatttcaagagaataatataatagaaccacaggtttttg

SEQ ID NO: 40 - protein, 468 aa, Artificial Sequence
  CD22-CAR
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 486 aa, Artificial Sequence
  CD19-CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 782 aa, Artificial Sequence
  Delta LNGFR_P2A_CD19-CAR
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWGSGATNFSLLKQAGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 21 bp, Artificial Sequence
  shRNA against TGFBR1
ggagattgttggtacccaagg

SEQ ID NO: 44 - DNA, 21 bp, Artificial Sequence
  shRNA against TGFBR1
gcagctaggcttacagcattg

SEQ ID NO: 45 - DNA, 21 bp, Artificial Sequence
  shRNA against TGFBR1
ggtcctttctgtgcactatga

SEQ ID NO: 46 - DNA, 21 bp, Artificial Sequence
  shRNA against TGFBR1
ggtggtagctaaagaacattc

SEQ ID NO: 47 - DNA, 21 bp, Artificial Sequence
  shRNA against TGFBR1
ggacctgtctacaggtgatct

SEQ ID NO: 48 - DNA, 22 bp, Artificial Sequence
  TGFBR1 primer Forward
acatgattcagccacagatacc

SEQ ID NO: 49 - DNA, 21 bp, Artificial Sequence
  TGFBR1 primer Reverse
gcatagatgtcagcacgtttg

SEQ ID NO: 50 - DNA, 396 bp, Artificial Sequence
  mU6-TIGIT shRNA
tctagacatatggctagcgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctgcatgactacttcaatgtttcaagagaacattgaagtagtcatgcagctttttgactagt

SEQ ID NO: 51 - DNA, 396 bp, Artificial Sequence
  mU6-TGFBR2 shRNA#1
tctagacatatggctagcgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggagaaagaatgacgagaacattcaagagatgttctcgtcattctttctcctttttgactagt

SEQ ID NO: 52 - DNA, 396 bp, Artificial Sequence
  mU6-TGFBR2 shRNA#3
tctagacatatggctagcgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggaggagaagattcctgaagattcaagagatcttcaggaatcttctcctcctttttgactagt

SEQ ID NO: 53 - DNA, 396 bp, Artificial Sequence
  mU6-TGFBR2 shRNA#4
tctagacatatggctagcgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcatagaggcgcctagaaattttcaagagaaatttctaggcgcctctatgctttttgactagt

SEQ ID NO: 54 - DNA, 396 bp, Artificial Sequence
  mU6-TGFBR2 shRNA#5
tctagacatatggctagcgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggttcctgtgtgcccttatttttcaagagaaaataagggcacacaggaacctttttgactagt

SEQ ID NO: 55 - DNA, 392 bp, Artificial Sequence
  mU6-TGFBR2 shRNA#7
tctagacatatggctagcgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttttcctgcatgagcaactgcattcaagagatgcagttgctcatgcaggatttttgactagt

SEQ ID NO: 56 - DNA, 392 bp, Artificial Sequence
  mU6-TGFBR2 shRNA#8
tctagacatatggctagcgatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcttctccaaagtgcattattcaagagataatgcactttggagaagctttttgactagt

SEQ ID NO: 57 - DNA, 26 bp, Artificial Sequence
  Primer #1
ggggatccccccatcagtccgcaaag

SEQ ID NO: 58 - DNA, 43 bp, Artificial Sequence
  Primer #2
aagttagtagctccgcttccccacctcttgaaggctatgtagg

SEQ ID NO: 59 - DNA, 87 bp, Artificial Sequence
  Primer #3
ggaagcggagctactaacttcagcctgctgaagcaggctggcgacgtggaggagaaccctggacctgccttaccagtgaccgccttg

SEQ ID NO: 60 - DNA, 23 bp, Artificial Sequence
  Primer #4
gttgattgtcgacttagcgaggg

SEQ ID NO: 61 - protein, 495 aa, Artificial Sequence
  EGFR-CAR
MALPVTALLLPLALLLHAARPDEVQLVESGGGLVQPGGSLRLSCAASGFSFTNYGVHWVRQAPGKGLEWVSVIWSGGNTDYNTSVKGRFTISRDNSKNTLYLQMNSLRAEDTAVYYCARALTYYDYEFAYWGQGTLVTVSSGGGGSGGGGSGGGGSGGGGSEIVLTQSPATLSLSPGERATLSCRASQSIGTNIHWYQQKPGQAPRLLIYYASESISGIPARFSGSGSGTDFTLTISSLEPEDFAVYYCQQNNNWPTTFGQGTKLEIKGSLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 251 aa, Artificial Sequence
  Cetuximab scFv
DEVQLVESGGGLVQPGGSLRLSCAASGFSFTNYGVHWVRQAPGKGLEWVSVIWSGGNTDYNTSVKGRFTISRDNSKNTLYLQMNSLRAEDTAVYYCARALTYYDYEFAYWGQGTLVTVSSGGGGSGGGGSGGGGSGGGGSEIVLTQSPATLSLSPGERATLSCRASQSIGTNIHWYQQKPGQAPRLLIYYASESISGIPARFSGSGSGTDFTLTISSLEPEDFAVYYCQQNNNWPTTFGQGTKLEIKGSLE

(19) United States
(12) Patent Application Publication
(10) Pub. No.: US 2022/0348928 A1
(43) Pub. Date: Nov. 3, 2022

(54) ENHANCED IMMUNE CELLS USING DUAL SHRNA AND COMPOSITION INCLUDING THE SAME

(71) Applicants: CUROCELL INC., Daejeon (KR); KOREA ADVANCED INSTITUTE OF SCIENCE AND TECHNOLOGY, Daejeon (KR)

(72) Inventors: Gun Soo KIM, Sejong-si (KR); Mi Kyung KIM, Daejeon (KR); Chan Hyuk KIM, Daejeon (KR); Young-Ho LEE, Daejeon (KR); HyeongJi LEE, Daejeon (KR)

(73) Assignees: CUROCELL INC., Daejeon (KR); KOREA ADVANCED INSTITUTE OF SCIENCE AND TECHNOLOGY, Daejeon (KR)

(21) Appl. No.: 17/626,768

(22) PCT Filed: Jul. 16, 2020
(86) PCT No.: PCT/IB2020/056680

§ 371 Date: Jan. 12, 2022

Related U.S. Application Data
(60) Provisional application No. 62/875,504, filed on Jul. 17, 2019.

(51) Int. Cl. C12N 15/113; C07K 14/725; C07K 14/705; C07K 16/28; A61K 35/17; A61P 35/00

(52) U.S. Cl. CPC C12N 15/1138; C07K 14/7051; C07K 14/70578; C07K 14/70521; C07K 16/2803; C07K 16/2863; A61K 35/17; A61P 35/00; C07K 2319/33; C07K 2319/03; C07K 2319/02; C12N 2310/122; C07K 2317/73; C12N 2320/31; C12N 2310/531; A61K 2039/5156

(57) ABSTRACT

The present disclosure is broadly concerned with the field of cancer immunotherapy. For example, the present invention generally relates to an immune cell comprising a genetically engineered antigen receptor that specifically binds to a target antigen and a genetic disruption agent that reduces or is capable of reducing the expression in the immune cell of two genes that weaken the function of the immune cell.

### CROSS REFERENCE TO RELATED APPLICATIONS

This application claims the benefit of U.S. Provisional Application No. 62/875,504, filed Jul. 17, 2019, which is incorporated by reference herein in its entirety.

### REFERENCE TO SEQUENCE LISTING SUBMITTED ELECTRONICALLY

This application incorporates by reference a Sequence Listing submitted with this application as a text file entitled 14570-002-228_SEQ_LISTING.txt, created on Jul. 13, 2020, and is 37,160 bytes in size.

### FIELD OF THE INVENTION

The present disclosure is broadly concerned with the field of cancer immunotherapy. For example, the present invention generally relates to an immune cell comprising a genetically engineered antigen receptor that specifically binds to a target antigen and a genetic disruption agent that reduces or is capable of reducing the expression in the immune cell of two genes that weaken the function of the immune cell.

### BACKGROUND

Anti-cancer therapies using immune cells by isolating T cells or NK cells (natural killer cells) from the body of a patient or a donor, culturing these cells in vitro, and then introducing them back into the body of a patient are currently receiving much attention as a new method of cancer therapy. In particular, immune cells having been subjected to a process of injecting new genetic information using viruses, etc. followed by culturing in an in vitro culturing process are reported to have greater anti-cancer effect over cells which have not. Here, the genetic information injected into the T cells is usually a Chimeric Antigen Receptor (hereinafter CAR) or a monoclonal T cell receptor (hereinafter mTCR) modified to have high affinity to the target antigen. These modified immune cells recognize and attack cancer cells which express the target antigen and induce cell death without being limited by their inherent antigen specificities. A method for genetically modifying T cells using CAR was first proposed by Eshhar et al. in 1989, and was called by the name of “T-body.”

Provided herein are immune cell compositions and methods which address the problems with conventional concurrent immune cell therapies pointed out above, wherein said problems place a great economic burden on patients due to their high cost, act on T cells other than CAR-T, and pose a risk of autoimmune symptoms and cytokine release syndrome. Briefly, for example, disclosed herein are methods of preparation with high yield rates and low production costs. Moreover, by inhibiting a combination of molecules that suppress the function of immune cells with higher probability and effectiveness, the disclosure herein meets the need for technology to provide effective cell therapy. The technical problem the present disclosure aims to solve is not limited to the technical problem stated above, and other technical problems not mentioned shall be evident from the following to persons having ordinary skill in the art.

### SUMMARY

Provided herein are vectors, immune cells, pharmaceutical compositions comprising the immune cells, and compositions comprising the immune cells. Also provided herein are methods of producing the immune cells, and methods of treatment and use of the immune cells.

In one aspect, provided herein is a vector, comprising: a base sequence encoding two types of short hairpin RNA (shRNA) which inhibit the expression of at least two genes that weaken the function of immune cells, and a base sequence encoding a chimeric antigen receptor (CAR) or a monoclonal T cell receptor (mTCR). In some embodiments, expression of the two types of shRNA is characterized in that they are regulated by two different promoters. In some embodiments, the two promoters are RNA polymerase III promoters. In other embodiments, the two promoters are U6 promoters derived from different species. In some embodiments, the two promoters are oriented in different directions from each other on the vector. In some embodiments, the genes weakening the function of immune cells are PD-1, TGFBR1, or TGFBR2. In specific embodiments, the genes weakening the function of immune cells are PD-1 and TGFBR1. In other specific embodiments, the genes weakening the function of immune cells are PD-1 and TGFBR2. In some embodiments, the genes weakening the function of immune cells are immune checkpoint receptors or ligands. In specific embodiments, the immune checkpoint receptor is PD1. In some embodiments, the two types of shRNA, one shRNA targets PD-1 and the second shRNA targets TGFBR1. In specific embodiments, the two types of shRNA, one shRNA targets PD-1 and the second shRNA targets TGFBR2. In more specific embodiments, the two types of shRNA, the base sequence encoding one shRNA comprises a sequence selected from a group consisting of SEQ ID NOs: 43-47, and the base sequence encoding the second shRNA comprises a different sequence selected from the group consisting of SEQ ID Nos: 13-23. In other specific embodiments, the two types of shRNA, the base sequence encoding one shRNA comprises a sequence selected from a group consisting of SEQ ID NOs: 1-12, and the base sequence encoding the second shRNA comprises a different sequence selected from the group consisting of SEQ ID Nos: 13-23.

In some embodiments, the target of the CAR or mTCR is a human tumor antigen that exhibits increased expression in a cancer cell, cancer tissue, and/or tumor microenvironment, or is a mutated form of antigen found in a cancer cell, cancer tissue and/or tumor microenvironment.

In some embodiments, the vector is a plasmid vector, a lentivirus vector, an adenovirus vector, an adeno-associated vector or a retrovirus vector.

In other aspects, provided herein is an immune cell comprising the vector of any of the preceding embodiments, wherein expression of at least one or two of the genes is reduced to 40% or less than that of expression in the absence of the shRNAs. In some embodiments, the immune cell is a human-derived T cell or natural killer (NK) cell.

In other aspects, provided herein is a pharmaceutical composition comprising the immune cell of any of the preceding embodiments. In some embodiments, the pharmaceutical composition is for treatment of a patient in need of immune therapy, wherein the immune cell is originally obtained from the patient. In some embodiments, the patient has a tumor or cancer in which the target, and/or an increase or variation in levels of the target, of the CAR or mTCR expressed in the immune cell is detected.

In another aspect, provided herein is an immune cell comprising a genetically engineered antigen receptor that specifically binds to a target antigen and a genetic disruption agent reducing or capable of reducing the expression in the immune cell of at least two genes that weaken the function of the immune cell. In some embodiments, the genetically engineered antigen receptor is a chimeric antigen receptor (CAR) or a T cell receptor (TCR). In specific embodiments, the genetically engineered antigen receptor is a CAR. In specific embodiments, the CAR comprises an extracellular antigen recognition domain, a transmembrane domain, and an intracellular signal transduction domain. In specific embodiments, the extracellular antigen recognition domain of the CAR specifically binds to the target antigen. In other specific embodiments, the intracellular signal transduction domain of the CAR comprises an intracellular domain of a CD3 zeta (CD3ζ) chain. In other specific embodiments, the intracellular signal transduction domain of the CAR further comprises a costimulatory molecule.

In some embodiments, the costimulatory molecule of the immune cell is selected from the group consisting of ICOS, 0X40, CD137 (4-1BB), CD27, and CD28. In specific embodiments, the costimulatory molecule is CD137 (4-1BB). In other specific embodiments, the costimulatory molecule is CD28. In some embodiments, the genetically engineered antigen receptor is a TCR. In specific embodiments, the TCR is a monoclonal TCR (mTCR).

In some embodiments, the target antigen is expressed in or on the surface of a cancer cell, a cancer tissue, and/or a tumor microenvironment. In specific embodiments, the target antigen is selected from the group consisting of: 5T4 (Trophoblast glycoprotein), 707-AP, 9D7, AFP (α-fetoprotein), AlbZIP (androgen-induced bZIP), HPG1 (human prostate specific gene-1), α5β1-Integrin, α5β6-Integrin, α-methylacyl-coenzyme A racemase, ART-4 (ADPribosyltransferase-4), B7H4 (v-set domain-containing T-cell activation inhibitor 1), BAGE-1 (B melanoma antigen-1), BCL-2 (B-cell CLL/lymphoma-2), BING-4 (WD repeat domain 46), CA 15-3/CA 27-29 (mucin 1), CA 19-9 (cancer antigen 19-9), CA 72-4 (cancer antigen 72-4), CA125 (cancer antigen 125), calreticulin, CAMEL (CTL-recognized antigen on melanoma), CASP-8 (caspase 8), cathepsin B, cathepsin L, CD19 (cluster of differentiation 19), CD20, CD22, CD25, CD30, CD33, CD4, CD52, CD55, CD56, CD80, CEA (carcinoembryonic antigen SG8), CLCA2 (chloride channel accessory 2), CML28 (chronic myelogenous leukemia tumor antigen 28), Coactosin-like protein, Collagen XXIII, COX-2 (cyclooxygenase-2), CT-9/BRD6 (cancer/testis antigen 9), Cten (c-terminal tensin-like protein), cyclin B1, cyclin D1, cyp-B, CYPB1 (cytochrome p450 family 1 subfamily b member 1), DAM-10/MAGE-B1 (melanoma-associated antigen B1), DAM-6/MAGE-B2, EGFR/Her1 (epidermal growth factor receptor), EMMPRIN (basigin), EpCam, EphA2 (EPH receptor A2), EphA3, ErbB3 (Erb-B2 receptor tyrosine kinase 3), EZH2 (enhancer of zeste 2 polycomb repressive complex 2 subunit), FGF-5 (fibroblast growth factor 5), FN (fibronectin), Fra-1 (Fosrelated antigen-1), G250/CAIX (carbonic anhydrase 9), GAGE-1 (G antigen-1), GAGE-2, GAGE-3, GAGE-4, GAGE-5, GAGE-6, GAGE-7b, GAGE-8, GDEP (gene differentially expressed in prostate), GnT-V (gluconate kinase), gp100 (melanocytes lineage-specific antigen GP100), GPC3 (glypican3), HAGE (helical antigen), HAST-2 (sulfotransferase family 1A member 1), hepsin, Her2/neu/ErbB2 (Erb-B2 receptor tyrosine kinase 2), HERV-K-MEL, HNE (medullasin), homeobox NKX 3.1, HOM-TES-14/SCP-1, HOM-TES-85, HPV-E6, HPVE7, HST-2 (sirtuin-2), hTERT, iCE (caspase 1), IGF-1R (insulin like growth factor-1 receptor), IL-13Ra2 (interleukin-13 receptor subunit a 2), IL-2R (interleukin-2 receptor), IL-5 (interleukin-5), immature laminin receptor, kallikrein 2, kallikrein 4, Ki67, KIAA0205 (lysophosphatidylglycerol acyltransferase 1), KK-LC-1 (kita-kyushu lung cancer antigen-1), KM-HN-1, LAGE-1 (L antigen family member-1), Livin, MAGE-A1, MAGE-A10, MAGE-A12, MAGEA2, MAGE-A3, MAGE-A4, MAGE-A6, MAGE-A9, MAGE-B1, MAGE-B10, MAGE-B16, MAGEB17, MAGE-B2, MAGE-B3, MAGE-B4, MAGE-B5, MAGE-B6, MAGE-C1, MAGE-C2, MAGE-C3, MAGE-D1, MAGE-D2, MAGE-D4, MAGE-E1, MAGE-E2, MAGE-F1, MAGE-H1, MAGEL2 (melanoma antigen family L2), mammaglobin A, MART-1/Melan-A (melanoma antigen recognized by T-cells-1), MART-2, matrix protein 22, MC1R (melanocortin 1 receptor), M-CSF (macrophage colony-stimulating factor), Mesothelin, MG50/PXDN (peroxidasin), MMP 11 (matrix metalloprotease 11), MN/CA IX-antigen (carbonic anhydrase 9), MRP-3 (multidrug resistance-associated protein-3), MUC1 (mucin 1), MUC2, NA88-A (VENT-like homeobox 2 pseudogene 1), N-acetylglucos-aminyltransferase-V, Neo-PAP (Neo-poly (A) polymerase), NGEP (new gene expressed in prostate), NMP22 (nuclear matrix protein 22), NPM/ALK (nucleophosmin), NSE (neuron-specific enolase), NY-ESO-1, NY-ESO-B, OA1 (osteoarthritis QTL 1), OFA-iLRP (oncofetal antigen immature laminin receptor protein), OGT (O-GlcNAc transferase), OS-9 (endoplasmic reticulum lectin), osteocalcin, osteopontin, p15 (CDK inhibitor 2B), p53, PAGE-4 (P antigen family member-4), PAI-1 (plasminogen activator inhibitor-1), PAI-2, PAP (prostatic acid phosphatase), PART-1 (prostate androgen-regulated transcript 1), PATE (prostate and testis expressed 1), PDEF (prostate-derived Ets factor), Pim-1-Kinase (proviral integration site 1), Pin1 (Peptidyl-prolyl cis-trans isomerase NIMA-interacting 1), POTE (expressed in prostate, ovary, testis, and placenta), PRAME (preferentially expressed antigen in melanoma), prostein, proteinase-3, PSA (prostate-specific antigen), PSCA (prostate stem cell antigen), PSGR (prostate-specific G-protein coupled receptor), PSM, PSMA (prostate specific membrane antigen), RAGE-1 (renal tumor carcinoma antigen), RHAMM/CD168, RU1 (renal ubiquitous protein 1), RU2, SAGE (sarcoma antigen), SART-1 (squamous cell carcinoma antigen recognized by T-cells-1), SART-2, SART-3, Sp17 (sperm protein 17), SSX-1 (SSX family member 1), SSX-2/HOM-MEL-40, SSX-4, STAMP-1 (STEAP2 metalloreductase), STEAP, survivin, survivin-213, TA-90 (tumor associated antigen-90), TAG-72 (tumor associated glycoprotein-72), TARP (TCRγ alternate reading frame protein), TGFb (transforming growth factor β), TGFbR11 (transforming growth factor β receptor 11), TGM-4 (transglutaminase 4), TRAG-3 (taxol resistance associated gene 3), TRG (T-cell receptor γ locus), TRP-1 (transient receptor potential-1), TRP-2/6b, TRP-2/INT2, Trp-p8, Tyrosinase, UPA (U-plasminogen activator), VEGF (vascular endothelial growth factor A), VEGFR-2/FLK-1, and WT1 (wilms tumor 1). In specific embodiments, the target antigen is CD19 or CD22. In specific embodiments, the target antigen is CD19. In specific embodiments, the target antigen is EGFR.

In some embodiments, the target antigen is a cancer antigen whose expression is increased in or on the surface of a cancer cell, a cancer tissue, and/or a tumor microenvironment. In specific embodiments, the target antigen is selected from the group consisting of: α-actinin-4/m, ARTC1/m, bcr/abl, beta-Catenin/m, BRCA1/m, BRCA2/m, CASP-5/m, CASP-8/m, CDC27/m, CDK4/m, CDKN2A/m, CML66, COA-1/m, DEK-CAN, EFTUD2/m, ELF2/m, ETV6-AML1, FN1/m, GPNMB/m, HLA-A*0201-R170I, HLA-All/m, HLA-A2/m, HSP70-2M, KIAA0205/m, K-Ras/m, LDLR-FUT, MART2/m, ME1/m, MUM-1/m, MUM-2/m, MUM-3/m, Myosin class 1/m, neo-PAP/m, NFYC/m, N-Ras/m, OGT/m, OS-9/m, p53/m, Pml/RARa, PRDX5/m, PTPRX/m, RBAF600/m, SIRT2/m, SYTSSX-1, SYT-SSX-2, TEL-AML1, TGFbRII, and TPI/m; and the target antigen is a cancer antigen that is a mutated form of antigen expressed in or on the surface of a cancer cell, a cancer tissue, and/or a tumor microenvironment.

In some embodiments of the immune cell of any of the preceding embodiments, expression of the genes that weaken the function of the immune cell causes one or more of the following: (i) inhibition of proliferation of the immune cell; (ii) induction of cell death of the immune cell; (iii) inhibition of the ability of the immune cell to recognize the target antigen and/or undergo activation; (iv) induction of differentiation of the immune cell into a cell that does not induce immune response to the target antigen; (v) decreased reactios of the immune cell to a molecule which promotes immune response of the immune cell; or (vi) increased reaction of the immune cell to a molecule which suppresses immune response of the immune cell.

In some embodiments, the genes that weaken the function of the immune cell are PD1, TGFBR1 or TGFBR2. In specific embodiments, the genes that weaken the function of the immune cell are PD1 and TGFBR1. In other specific embodiments, the genes that weaken the function of the immune cell are PD1 and TGFBR2.

In some embodiments, at least one or two of the genes that weaken the function of the immune cell increase reaction of the immune cell to a molecule which suppresses immune response of the immune cell. In some embodiments, at least one or two of the genes that increase reaction of the immune cell to a molecule which suppresses immune response of the immune cell encode an immune checkpoint receptor or ligand. In some embodiments, the immune checkpoint receptor is PD1.

In some embodiments, of the immune cell of the preceding embodiments, the genetic disruption agent reduces the expression of at least two genes in the immune cell that weaken the function of the immune cell by at least 30, 40, 50, 60, 70, 80, 90, or 95% as compared to the immune cell in the absence of the genetic disruption agent. In certain aspects, a genetic disruption agent that reduces the expression of at least two genes comprises at least a first portion that reduces the expression of a first gene and a second portion that reduces the expression of a second gene. In certain aspects, a genetic disruption agent that reduces the expression of two genes comprises a first portion that reduces the expression of a first gene and a second portion that reduces the expression of a second gene.

In some embodiments, the genetic disruption agent reduces the expression of at least two genes that increases reaction of the immune cell to a molecule which suppresses immune response of the immune cell. In some embodiments, the genetic disruption agent reduces the expression of at least two genes that encodes an immune checkpoint receptor or ligand. In some embodiments, the genetic disruption agent reduces the expression of at least one of PD1, TGFBR1 or TGFBR2. In some embodiments, the genetic disruption agent reduces the expression of at least two of PD1, TGFBR1, or TGFBR2. In some embodiments, the genetic disruption agent reduces the expression of at least PD1 and TGFBR1. In some embodiments, the genetic disruption agent reduces the expression of at least PD1 and TGFBR2. In specific embodiments, the genetic disruption agent reduces the expression of genes that weaken the function of the immune cell by RNA interference (RNAi).

In some embodiments of the immune cell of the preceding embodiments, the immune cell comprises more than one genetic disruption agent that reduces the expression of genes that weaken the function of the immune cell in the immune cell by RNAi. In some embodiments, different genetic disruption agents target different genes which weaken the function of the immune cell, for example, wherein a first genetic disruption agent targets a first gene and a second genetic disruption agent targets a second gene. In some embodiments, the RNAi is mediated by a short hairpin RNA (shRNA). In some embodiments, the RNAi is mediated by more than one shRNA. In some embodiments, the RNAi is mediated by two shRNAs. In some embodiments, a first shRNA targets PD-1 and a second shRNA targets TGFBR1. In other embodiments, a first shRNA targets PD-1 and a second shRNA targets TGFBR2.

In some embodiments of the immune cell of the preceding embodiments, the immune cell comprises nucleotide sequences that encode more than one shRNA. In some embodiments, the immune cell comprises nucleotide sequences that encode two shRNAs. In specific embodiments, the nucleotide sequence encoding the two shRNAs comprises a nucleotide sequence selected from the group consisting of SEQ ID NOs.: 1-12 and SEQ ID NOs.: 13-23, respectively. In other specific embodiments, the two nucleotide sequence encoding the shRNAs comprises a nucleotide sequence selected from the group consisting of SEQ ID NOs.: 43-47 and SEQ ID NOs.: 13-23, respectively.

In some embodiments of the immune cell of the preceding embodiments, the nucleotide sequence encoding the shRNA(s) is present on a vector. In some embodiments, the expression of two different shRNA is regulated by two different promoters. In specific embodiments, the two different promoters are RNA polymerase III promoters. In other specific embodiments, the two promoters are U6 promoters. In some embodiments, the U6 promoters derived from different species. In some embodiments, the two promoters are oriented in different directions from each other.

In some embodiments of the immune cell of the preceding embodiments, the genetically engineered antigen receptor and the genetic disruption agent(s) are each expressed from a vector. In some embodiments, the genetically engineered antigen receptor and the genetic disruption agent(s) are expressed from the same vector. In some embodiments, the vector is a plasmid vector or a viral vector. In some embodiments, the viral vector is a lentivirus vector, adenovirus vector or adeno-associated viral vector. In specific embodiments, the lentivirus vector is a retrovirus vector.

In some embodiments of the immune cell of the preceding embodiments, the immune cell is selected from the group consisting of a T cell and a natural killer (NK) cell. In specific embodiments, the immune cell is a T cell. In more specific embodiments, the T cell is a CD4+ T cell or a CD8+ T cell.

In some embodiments of the immune cell of the preceding embodiments, the immune cell comprises nucleotide sequences that encode two shRNAs and a CAR or mTCR on the same vector. In some embodiments, the two shRNAs are each regulated by two different RNA polymerase III promoters. In some embodiments, the two shRNAs are each regulated by two different RNA polymerase III promoters oriented in different directions from each other. In specific embodiments, the CAR targets CD19, the first shRNA targets PD-1, and the second shRNA targets TGFBR1. In other specific embodiments, the CAR targets CD19, the first shRNA targets PD-1, and the second shRNA targets TGFBR2. In other specific embodiments, the CAR targets EGFR, the first shRNA targets PD-1, and the second shRNA targets TGFBR1. In other specific embodiments, the CAR targets EGFR, the first shRNA targets PD-1, and the second shRNA targets TGFBR2.

In another aspect, provided herein is a method of producing an immune cell comprising introducing into an immune cell, simultaneously or sequentially in any order: (1) a gene encoding a genetically engineered antigen receptor that specifically binds to a target antigen; and (2) a genetic disruption agent, wherein the genetic disruption agent, or expression thereof, reduces or is capable of reducing expression in the immune cell of at least two genes that weaken the function of the immune cell, thereby producing an immune cell in which a genetically engineered antigen receptor is expressed and expression of two genes that weaken the function of the immune cell is reduced.

In some embodiments, the genetically engineered antigen receptor is a chimeric antigen receptor (CAR) or a T cell receptor (TCR). In specific embodiments, the genetically engineered antigen receptor is a CAR. In some embodiments, the CAR comprises an extracellular antigen recognition domain, a transmembrane domain, and an intracellular signal transduction domain. In specific embodiments, the extracellular antigen recognition domain of the CAR specifically binds to the target antigen. In some embodiments, the intracellular signal transduction domain of the CAR comprises an intracellular domain of a CD3 zeta (CD3ζ) chain. In some embodiments, the intracellular signal transduction domain of the CAR further comprises a costimulatory molecule. In some embodiments, the costimulatory molecule is selected from the group consisting of ICOS, 0X40, CD137 (4-1BB), CD27, and CD28. In specific embodiments, the costimulatory molecule is CD137 (4-1BB). In other specific embodiments, the costimulatory molecule is CD28.

In some embodiments of the method of the preceding embodiments, the genetically engineered antigen receptor is a TCR. In some embodiments, the TCR is a monoclonal TCR (mTCR). In some embodiments, the target antigen is expressed in or on the surface of a cancer cell, a cancer tissue, and/or a tumor microenvironment. In some embodiments, the target antigen is selected from the group consisting of: 5T4 (Trophoblast glycoprotein), 707-AP, 9D7, AFP (α-fetoprotein), AlbZIP (androgen-induced bZIP), HPG1 (human prostate specific gene-1), α5β1-Integrin, α5β6-Integrin, a -methylacyl-coenzyme A racemase, ART-4 (ADPribosyltransferase-4), B7H4 (v-set domain-containing T-cell activation inhibitor 1), BAGE-1 (B melanoma antigen-1), BCL-2 (B-cell CLL/lymphoma-2), BING-4 (WD repeat domain 46), CA 15-3/CA 27-29 (mucin 1), CA 19-9 (cancer antigen 19-9), CA 72-4 (cancer antigen 72-4), CA125 (cancer antigen 125), calreticulin, CAMEL (CTL-recognized antigen on melanoma), CASP-8 (caspase 8), cathepsin B, cathepsin L, CD19 (cluster of differentiation 19), CD20, CD22, CD25, CD30, CD33, CD4, CD52, CD55, CD56, CD80, CEA (carcinoembryonic antigen SG8), CLCA2 (chloride channel accessory 2), CML28 (chronic myelogenous leukemia tumor antigen 28), Coactosin-like protein, Collagen XXIII, COX-2 (cyclooxygenase-2), CT-9/BRD6 (cancer/testis antigen 9), Cten (c-terminal tensin-like protein), cyclin B1, cyclin D1, cyp-B, CYPB1 (cytochrome p450 family 1 subfamily b member 1), DAM-10/MAGE-B1 (melanoma-associated antigen B1), DAM-6/MAGE-B2, EGFR/Her1 (epidermal growth factor receptor), EMMPRIN (basigin), EpCam, EphA2 (EPH receptor A2), EphA3, ErbB3 (Erb-B2 receptor tyrosine kinase 3), EZH2 (enhancer of zeste 2 polycomb repressive complex 2 subunit), FGF-5 (fibroblast growth factor 5), FN (fibronectin), Fra-1 (Fosrelated antigen-1), G250/CAIX (carbonic anhydrase 9), GAGE-1 (G antigen-1), GAGE-2, GAGE-3, GAGE-4, GAGE-5, GAGE-6, GAGE-7b, GAGE-8, GDEP (gene differentially expressed in prostate), GnT-V (gluconate kinase), gp100 (melanocytes lineage-specific antigen GP100), GPC3 (glypican3), HAGE (helical antigen), HAST-2 (sulfotransferase family 1A member 1), hepsin, Her2/neu/ErbB2 (Erb-B2 receptor tyrosine kinase 2), HERV-K-MEL, HNE (medullasin), homeobox NKX 3.1, HOM-TES-14/SCP-1, HOM-TES-85, HPV-E6, HPVE7, HST-2 (sirtuin-2), hTERT, iCE (caspase 1), IGF-1R (insulin like growth factor-1 receptor), IL-13Ra2 (interleukin-13 receptor subunit a 2), IL-2R (interleukin-2 receptor), IL-5 (interleukin-5), immature laminin receptor, kallikrein 2, kallikrein 4, Ki67, KIAA0205 (lysophosphatidylglycerol acyltransferase 1), KK-LC-1 (kita-kyushu lung cancer antigen-1), KM-HN-1, LAGE-1 (L antigen family member-1), Livin, MAGE-A1, MAGE-A10, MAGE-A12, MAGEA2, MAGE-A3, MAGE-A4, MAGE-A6, MAGE-A9, MAGE-B1, MAGE-B10, MAGE-B16, MAGEB17, MAGE-B2, MAGE-B3, MAGE-B4, MAGE-B5, MAGE-B6, MAGE-C1, MAGE-C2, MAGE-C3, MAGE-D1, MAGE-D2, MAGE-D4, MAGE-E1, MAGE-E2, MAGE-F1, MAGE-H1, MAGEL2 (melanoma antigen family L2), mammaglobin A, MART-1/Melan-A (melanoma antigen recognized by T-cells-1), MART-2, matrix protein 22, MC1R (melanocortin 1 receptor), M-CSF (macrophage colony-stimulating factor), Mesothelin, MG50/PXDN (peroxidasin), MMP 11 (matrix metalloprotease 11), MN/CA IX-antigen (carbonic anhydrase 9), MRP-3 (multidrug resistance-associated protein-3), MUC1 (mucin 1), MUC2, NA88-A (VENT-like homeobox 2 pseudogene 1), N-acetylglucos-aminyltransferase-V, Neo-PAP (Neo-poly (A) polymerase), NGEP (new gene expressed in prostate), NMP22 (nuclear matrix protein 22), NPM/ALK (nucleophosmin), NSE (neuron-specific enolase), NY-ESO-1, NY-ESO-B, OA1 (osteoarthritis QTL 1), OFA-iLRP (oncofetal antigen immature laminin receptor protein), OGT (O-GlcNAc transferase), OS-9 (endoplasmic reticulum lectin), osteocalcin, osteopontin, p15 (CDK inhibitor 2B), p53, PAGE-4 (P antigen family member-4), PAI-1 (plasminogen activator inhibitor-1), PAI-2, PAP (prostatic acid phosphatase), PART-1 (prostate androgen-regulated transcript 1), PATE (prostate and testis expressed 1), PDEF (prostate-derived Ets factor), Pim-l-Kinase (proviral integration site 1), Pin1 (Peptidyl-prolyl cis-trans isomerase NIMA-interacting 1), POTE (expressed in prostate, ovary, testis, and placenta), PRAME (preferentially expressed antigen in melanoma), prostein, proteinase-3, PSA (prostate-specific antigen), PSCA (prostate stem cell antigen), PSGR (prostate-specific G-protein coupled receptor), PSM, PSMA (prostate specific membrane antigen), RAGE-1 (renal tumor carcinoma antigen), RHAMM/CD168, RU1 (renal ubiquitous protein 1), RU2, SAGE (sarcoma antigen), SART-1 (squamous cell carcinoma antigen recognized by T-cells-1), SART-2, SART-3, Sp17 (sperm protein 17), SSX-1 (SSX family member 1), SSX-2/HOM-MEL-40, SSX-4, STAMP-1 (STEAP2 metalloreductase), STEAP, survivin, survivin-213, TA-90 (tumor associated antigen-90), TAG-72 (tumor associated glycoprotein-72), TARP (TCRγ alternate reading frame protein), TGFb (transforming growth factor β), TGFbR11 (transforming growth factor β receptor 11), TGM-4(transglutaminase 4), TRAG-3 (taxol resistance associated gene 3), TRG (T-cell receptor y locus), TRP-1 (transient receptor potential-1), TRP-2/6b, TRP-2/INT2, Trp-p8, Tyrosinase, UPA (U-plasminogen activator), VEGF (vascular endothelial growth factor A), VEGFR-2/FLK-1, and WT1 (wilms tumor 1). In specific embodiments, the target antigen is CD19 or CD22. In specific embodiments, the target antigen is CD19. In specific embodiments, the target antigen is EGFR.

In some embodiments, the target antigen is a cancer antigen, wherein the cancer antigen is an antigen whose expression is increased in or on the surface of a cancer cell, a cancer tissue, and/or a tumor microenvironment. In specific embodiments, the target antigen is selected from the group consisting of: a-actinin-4/m, ARTC1/m, bcr/abl, beta-Catenin/m, BRCA1/m, BRCA2/m, CASP-5/m, CASP-8/m, CDC27/m, CDK4/m, CDKN2A/m, CML66, COA-1/m, DEK-CAN, EFTUD2/m, ELF2/m, ETV6-AML1, FN1/m, GPNMB/m, HLA-A*0201-R170I, HLA-A11/m, HLA-A2/m, HSP70-2M, KIAA0205/m, K-Ras/m, LDLR-FUT, MART2/m, ME1/m, MUM-1/m, MUM-2/m, MUM-3/m, Myosin class 1/m, neo-PAP/m, NFYC/m, N-Ras/m, OGT/m, OS-9/m, p53/m, Pml/RARa, PRDX5/m, PTPRX/m, RBAF600/m, SIRT2/m, SYTSSX-1, SYT-SSX-2, TEL-AML1, TGFbRII, and TPI/m; and the target antigen is a cancer antigen, wherein the cancer antigen is a mutated form of antigen expressed in or on the surface of a cancer cell, a cancer tissue, and/or a tumor microenvironment.

In some embodiments of the method of the preceding embodiments, expression of genes that weaken the function of the immune cell causes one or more of the following: (i) inhibition of proliferation of the immune cell; (ii) induction of cell death of the immune cell; (iii) inhibition of the ability of the immune cell to recognize the target antigen and/or to get activated; (iv) induction of differentiation of the immune cell into a cell that does not induce immune response to the target antigen; (v) decreased reaction of the immune cell to a molecule which promotes immune response of the immune cell; or (vi) increased reaction of the immune cell to a molecule which suppresses immune response of the immune cell.

In some embodiments, at least one or two of the genes that weaken the function of the immune cell are PD1, TGFBR1, or TGFBR2. In some embodiments, the genes that weaken the function of the immune cell comprise PD1 and TGFBR1. In some embodiments, the genes that weaken the function of the immune cell comprise PD1 and TGFBR2. In some embodiments, at least one or two of the genes that weaken the function of the immune cell increase reaction of the immune cell to a molecule which suppresses immune response of the immune cell. In some embodiments, at least one or two of the genes that increase reaction of the immune cell to a molecule which suppresses immune response of the immune cell encode an immune checkpoint receptor and ligand. In specific embodiments, the immune checkpoint receptor ligand is PD1.

In some embodiments of the method of the preceding embodiments, the genetic disruption agent reduces the expression of at least two genes in the immune cell that weaken the function of the immune cell by at least 30, 40, 50, 60, 70, 80, 90, or 95% as compared to the immune cell in the absence of the genetic disruption agent(s). In some embodiments, the genetic disruption agent reduces the expression of at least two genes that increase reaction of the immune cell to a molecule which suppresses immune response of the immune cell. In some embodiments, the genetic disruption agent reduces the expression of at least two genes that encode an immune checkpoint receptor or ligand. In specific embodiments, the genetic disruption agent reduces the expression of PD1 and TGFBR1. In other specific embodiments, the genetic disruption agent reduces the expression of PD1 and TGFBR2.

In some embodiments, the genetic disruption agent reduces the expression of genes that weaken the function of the immune cell by RNA interference (RNAi). In some embodiments, more than one genetic disruption agents reduce the expression of genes that weaken the function of the immune cell in the immune cell by RNAi. In some embodiments, the genetic disruption agents target different genes which weaken the function of the immune cell wherein a first genetic disruption agent targets a first gene and a second genetic disruption agent targets a second gene. In some embodiments, the RNAi is mediated by a short hairpin RNA (shRNA). In some embodiments, the RNAi is mediated by more than one shRNA. In some embodiments, the RNAi is mediated by two shRNAs. In specific embodiments, a first shRNA targets PD-1 and a second shRNA targets TGFBR1. In other specific embodiments, a first shRNA targets PD-1 and a second shRNA targets TGFBR2.

In some embodiments of the method of the preceding embodiments, the immune cell comprises nucleotide sequences that encode a shRNA. In some embodiments, the immune cell comprises nucleotide sequences that encode more than one shRNA. In some embodiments, the immune cell comprises nucleotide sequences that encode two shRNAs. In specific embodiments, the two nucleotide sequences encoding the shRNAs comprise sequences selected from the groups consisting of SEQ ID NOs.: 1-12 and SEQ ID NOs.: 13-23, respectively. In other specific embodiments, the two nucleotide sequences encoding the shRNAs comprise sequences selected from the groups consisting of SEQ ID NOs.: 43-47 and SEQ ID NOs.: 13-23, respectively.

In some embodiments, the nucleotide sequences encoding the shRNA is present on a vector. In some embodiments, the expression of different shRNAs is respectively regulated by different promoters. In some embodiments, the expression of two different shRNAs is respectively regulated by two different promoters. In some embodiments, the two different promoters are RNA polymerase III promoters. In other embodiments, the two promoters are U6 promoters. In some embodiments, the U6 promoters derive from different species. In some embodiments the two promoters are oriented in different directions from each other.

In some embodiments, the genetically engineered antigen receptor and the genetic disruption agent(s) are each expressed from a vector. In some embodiments, the genetically engineered antigen receptor and the genetic disruption agent(s) are expressed from the same vector. In some embodiments, the vector is a plasmid vector or a viral vector. In some embodiments, the viral vector is a lentivirus vector, adenovirus vector or adeno-associated viral vector. In specific embodiments, the lentivirus vector is a retrovirus vector.

In some embodiments of the method of the preceding embodiments, the immune cell is selected from the group consisting of a T cell and a natural killer (NK) cell. In other embodiments, the immune cell is a T cell. In specific embodiments, the T cell is a CD4+ T cell or a CD8+ T cell.

In some embodiments of the method of the preceding embodiments, the immune cell comprises nucleotide sequences that encode two shRNAs and a CAR on the same vector. In some embodiments, the two shRNAs are each regulated by two different RNA polymerase III promoters. In some embodiments, the two shRNAs are each regulated by two different RNA polymerase III promoters oriented in different directions from each other. In specific embodiments, the CAR targets CD19, the first shRNA targets PD-1, and the second shRNA targets TGFBR1. In other specific embodiments, the CAR targets CD19, the first shRNA targets PD-1, and the second shRNA targets TGFBR2. In other specific embodiments, the CAR targets EGFR, the first shRNA targets PD-1, and the second shRNA targets TGFBR1.

In other specific embodiments, the CAR targets EGFR, the first shRNA targets PD-1, and the second shRNA targets TGFBR2.

In another aspect, provided herein is a composition comprising the immune cell of any of the preceding embodiments. In another aspect, provided herein is a pharmaceutical composition comprising the immune cell of any of the preceding embodiments, and a pharmaceutically acceptable carrier.

In another aspect, provided herein is a method of treatment comprising administering to a subject having a disease or condition in need of immune therapy the immune cell or the composition of any of the preceding embodiments. In some embodiments, the genetically engineered antigen receptor specifically binds to an antigen associated with the disease or the condition. In specific embodiments, the disease or the condition is a cancer, e.g., a tumor. In exemplary embodiments, the cancer is non-Hodgkin's lymphoma.

In another aspect, provided herein is an immune cell or a composition of any of the preceding embodiments for use in treating a disease or a condition. In another aspect, provided herein is the use of the immune cell or the composition of any of the preceding embodiments in the manufacture of a medicament for treating a disease or a condition. In some embodiments, the genetically engineered antigen receptor specifically binds to an antigen associated with the disease or the condition. In specific embodiments, the disease or the condition is a cancer, e.g. a tumor.

### BRIEF DESCRIPTION OF THE DRAWINGS

FIGS. 1A-1D. Diagram of the composition of two orientations of vectors encoding two types of shRNA one of which inhibits the expression of PD-1 and the second of which inhibits the expression of TGFBR1, and a CD19 CAR expression cassette (FIG. 1A and FIG. 1B) or vectors encoding two types of shRNA one of which inhibits the expression of PD-1 and the second of which inhibits the expression of TGFBR2, and a CD19 CAR expression cassette (FIG. 1C and FIG. 1D)

FIGS. 2A-2B. Diagram of the CAR-T cell preparation process, wherein ΔLNGFR-CART19/mU6-shTGFBR1→←shPD-1-hU6 cells and ΔLNGFR-CART19/shTGFBR1-mU6←→hU6-shPD-1 cells (FIG. 2A) or ΔLNGFR-CART19/mU6-shTGFBR2→←shPD-1-hU6 cells and ΔLNGFR-CART19/shTGFBR2-mU6←→hU6-shPD-1 cells (FIG. 2B) were prepared and isolated as described herein.

FIGS. 3A-3C. DNA structure of EGFR-specific dual two-in-one vectors.

FIG. 4. EGFR-specific CAR and ΔLNGFR expression of transduced T cells. CAR-T cells were sorted by using LNGFR magnetic beads and cultured for 6 days. ΔLNGFR and EGFR-CAR expression were evaluated using APC conjugated anti-LNGFR antibody and Biotinylated human EGFR protein.

FIG. 5. Homeostatic expansion of CAR T cells. CAR transduced T cells were sorted by using LNGFR magnetic beads and seeded at 2×10⁵/ml. Cumulative CAR T cell counts were assessed by trypan blue staining. * indicates a p-value of <0.05.

FIG. 6. Evaluation of PD-1 knock-down efficiency. To determine the PD-1 protein level of CART cells, CART cells were stimulated with T Cell TransAct™ reagent. After 48 hours, PD-1 expression of CART cells was assessed using APC conjugated anti-LNGFR antibody and PE-conjugated anti-PD-1 antibody.

FIGS. 7A and 7B. Evaluation of TGFBR2 knock-down efficiency. Western blotting was performed to detect the expression of TGFBR2 in 48 hr stimulated CART cells with anti-human TGFBR2 antibody. A second trial (FIG. 7B) was performed to due to the ambiguity seen in the first trial (FIG. 7A).

FIG. 8. Phosphorylation of SMAD2. To detect the phosphorylation of SMAD2, Western blotting was performed using whole-protein lysate of CART cells treated with 5 ng/mL recombinant human TGF-β1 for 48 hr. SMAD2 and phospho-SMAD2 was detected with SMAD2(D43B4) and pSMAD2(138D4) specific antibodies.

FIG. 9. Expression of Inhibitory ligands in MDA-MB-231-Zsgreen. Zsgreen expression was evaluated by comparing MDA-MB-231 and MDA-MB-231-Zsgreen using flow cytometry. To evaluate the expression level of EGFR or inhibitory ligands, PE-conjugated anti-human EGFR, APC-conjugated anti-human PDL1, PE-conjugated anti-CD155, PE-conjugated anti-CD112, PE-conjugated anti-CD80, or PE-conjugated anti-CD86 antibodies were used. To detect the expression of intracellular galectin-9, intracellular staining procedure was conducted using PerCP-Cy5.5-conjugated anti-Galectin-9 antibody.

FIGS. 10A-10C. Optimal concentration of TGF-β1 for in vitro functional analysis. 1×10⁵ MDA-MB-231-Zsgreen cells were seeded in 96-well flat-bottom plates. After 24 hr, CAR T cells were added at E: T ratios of 1:1 (FIG. 10A), 0.3:1 (FIG. 10B) or 0.1:1 (FIG. 10C) in the absence or presence of 25 or 5 ng/mL recombinant human TGF-β1. CD19 specific CART cells were used as control CART. GFP fluorescent intensity per well was detected every 2 hr. Total integrated GFP intensity per well was as a quantitative measure of viable target cells. Values of total integrated GFP intensity were normalized to the starting point (zero hours).

FIGS. 11A-11D. Incucyte-based cytotoxicity. 1×10⁵ MDA-MB-231-Zsgreen cells were seeded in 96-well flat-bottom plates. After 24 hr, PD-1 and TGFBR2 KD CAR T cells were added at E: T ratios of 1:1 (FIGS. 11A and 11C) or 0.3:1 (FIGS. 11B and 11D) in the absence (FIGS. 11A and 11B) or presence (FIGS. 11C and 11D) of 5 ng/mL recombinant human TGF-β1. Fluorescent intensity per well was measured every 2 hr using the IncuCyte S3 Live-Cell analysis system (Sartorius, Germany) and collected at each time point. Total integrated GFP intensity per well was as a quantitative measure of viable target cells. Values of total integrated GFP intensity were normalized to the starting point.

FIGS. 12A and 12B. Multi-round antigen stimulation assay. 1×10⁵ MDA-MB-231-Zsgreen cells were seeded in 96-well flat-bottom plates. After 24 hr, 1×10⁵ CAR T cells were incubated in the absence or presence of 5 ng/mL recombinant human TGF-β1. The new 2×10⁵ MDA-MB-231-Zsgreen cells were added every 2 days. The final concentration of TGF-β1 was maintained at 5 ng/mL in the addition group. 8 days after the addition of CAR T cells, The percentage of LNGFR⁺ or Zsgreen⁺ cells was determined by flow cytometry. Fold change of CAR-T cells was calculated using a ratio [number of live CAR T cells at day 8/numbers of live CAR T cells at day 8]. Relative fold change of CAR T cells was calculated using a ratio [Fold change of TGF-β1 treated CART cells/Fold change of untreated CART cells]. FIG. 12A shows flow cytometry data which is quantified in FIG. 12B.

FIGS. 13A and 13B. EGFR specific CAR and ΔLNGFR expression of PD-1/TGFBR2, TIM-3/TGFBR2, or TIGIT/TGFBR2 KD CART cells. CAR-T cells were sorted by using LNGFR magnetic beads and incubated for 6 days. ΔLNGFR and EGFR-CAR expression were evaluated by flow cytometry using APC conjugated anti-LNGFR antibody and Biotinylated human EGFR protein. FIG. 13A shows flow cytometry data which is quantified in FIG. 13B.

FIG. 14. Homeostatic expansion of PD-1/TGFBR2, TIM-3/TGFBR2, or TIGIT/TGFBR2 KD CART cells. Transduced T cells were sorted by using LNGFR magnetic beads and seeded at 2×10⁵/ml. Cumulative CAR T cell counts were assessed by trypan blue staining at day 6 after cell seeding.

FIGS. 15A and 15B. Evaluation of PD-1, TIM-3, or TIGIT knock-down efficiency. To determine the PD-1, TIM-3, or TIGIT protein level of CART cells, CART cell were stimulated with T Cell TransAct™. After 48 hours, PD-1, TIM-3, or TIGIT expression of CART cells was assessed using APC conjugated anti-LNGFR, PE-conjugated anti-PD-1, PE-conjugated TIM-3, or PE-conjugated TIGIT antibody. FIG. 15A shows flow cytometry data, which is quantified in FIG. 15B

FIG. 16. Evaluation of TGFBR2 knock-down efficiency. Western blotting was performed to detect the expression of TGFBR2 of 48 hr stimulated PD-1/TGFBR2, TIM-3/TGFBR2, or TIGIT/TGFBR2 KD CART cells with anti-human TGFBR2 antibody.

FIGS. 17A and 17B. Multi-round antigen stimulation assay. 1×10⁵ MDA-MB-231-Zsgreen cells were seeded in 96-well flat-bottom plates. After 24 hr, 1×10⁵ WT, PD-1/TGFBR2, TIM-3/TGFBR2, or TIGIT/TGFBR2 KD CART cells were incubated in the absence or presence of 5 ng/mL recombinant human TGF-β1. The new 2×10⁵ MDA-MB-231-Zsgreen cells were added every 2 days. The final concentration of TGF-β1 was maintained at 5 ng/mL in the addition group. 8 days after the addition of CAR T cells, The percentage of LNGFR⁺ or Zsgreen⁺ cells was determined by flow cytometry. Fold change of CAR-T cells was calculated using a ratio [number of live CAR T cells at day 8/numbers of live CAR T cells at day 8]. Relative fold change of CAR T cells was calculated using a ratio [Fold change of TGF-β1 treated CAR T cells/ Fold change of untreated CAR T cells]. FIG. 17A shows flow cytometry data which is quantified in FIG. 17B.

### DETAILED DESCRIPTION

The features of the present disclosure are set forth specifically in the appended claims. A better understanding of the features and benefits of the present disclosure will be obtained by reference to the following detailed description that sets forth illustrative embodiments, in which the principles of the disclosure are utilized. To facilitate a full understanding of the disclosure set forth herein, a number of terms are defined below.

Briefly, in one aspect, disclosed herein are vectors comprising: a base sequence encoding two different types of short hairpin RNA (shRNA) which inhibit the expression of at least two genes that weaken the function of immune cells, including immune checkpoint receptors and ligands, and a base sequence encoding an antigen receptor such as a chimeric antigen receptor (CAR) or a T cell receptor (TCR), for example, a monoclonal T cell receptor (mTCR). In another aspect, disclosed herein is an immune cell comprising a genetically engineered antigen receptor that specifically binds to a target antigen and two different types of genetic disruption agents that reduce or are capable of reducing the expression in the immune cell of at least two different genes that weaken the function of the immune cell. In another aspect, disclosed herein are methods of producing the immune cell; a composition or pharmaceutical composition comprising the immune cell, e.g., for immune therapy of human patients; and a method of treatment comprising administering the immune cell to a subject having a disease or a condition. As the immune cell, composition, or pharmaceutical composition comprises two types of genetic disruption agents, e.g., encodes two shRNAs that reduce the expression of two immune checkpoint molecule genes which may be activated by cancer cells to weaken the function of immune cells, it is possible to eliminate severe and systemic adverse reactions such as cytokine release syndrome or autoimmune symptoms which can result from use of a separate inhibitor for these genes, as well as reducing the burden due to the increased cost of treatment resulting from expensive concurrent therapies, while providing cell therapy more effective than cases where only one shRNA is expressed.

1. General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (4th ed. 2012); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2nd ed. 2010). Molecular Biology of the Cell (6th Ed., 2014).

2. Definitions

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

The terms used in the present disclosure are used only to explain specific embodiments, and are not intended to limit the scope of the present invention. Singular expressions, unless clearly indicated otherwise by context, include plural expressions. It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Reference throughout this specification to “one embodiment,” “an embodiment,” “a particular embodiment,” “a related embodiment,” “a certain embodiment,” “an additional embodiment,” or “a further embodiment” or combinations thereof or the like means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A “construct” refers to a macromolecule or complex of molecules comprising a polynucleotide to be delivered to a target cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

A “two-in-one vector,” as described herein is a vector that comprises a base sequence encoding one or more short hairpin RNAs (shRNAs) which inhibit the expression of a gene or genes that weaken the function of immune cells, and a base sequence encoding a chimeric antigen receptor (CAR) or a T cell receptor, e.g., a monoclonal T cell receptor (mTCR). A “dual two-in-one vector” as described herein is a vector that comprises a base sequence encoding two types of short hairpin RNA (shRNA) which inhibit the expression of genes that weaken the function of immune cells, and a base sequence encoding any one of a chimeric antigen receptor (CAR) and T cell receptor, e.g., monoclonal T cell receptor (mTCR). Dual two-in-one vectors described herein are a form of two-in-one vector.

“RNAi” (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs and that match the nucleic acid sequence of the target gene. These short RNA species may be naturally produced in vivo by Dicer-mediated cleavage of larger dsRNAs and they are functional in mammalian cells. DNA expression plasmids can be used to stably express the siRNA duplexes or dsRNA of the present disclosure in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

The term “shRNA” refers an RNA molecule wherein some self-complementary sequences create a tight hairpin structure with its stem. The RNA molecule can have a length of approximately 80 bp. When shRNA is expressed in a cell, it is processed through a series of steps to become small interfering RNA (siRNA) which acts as a guide for gene silencing Simply put, when shRNA is expressed, it is processed by Drosha complexes in the cell to become pre-shRNA, which is then transported outside the nucleus where it undergoes further processing by a Dicer to become siRNA, and then is single stranded and loaded by an RISC (RNA-induced silencing complex) complex. Here, the antisense strand of siRNA acts as a guide for the RISC complex to attach to the mRNA of the target gene, and gene silencing occurs when the RISC complex, which has attached in this manner, cuts the mRNA. As shRNA in a target gene allows for gene silencing which is lasting and specific to a certain gene, it is included in the vector for the purpose of inhibiting the target gene.

The term “promoter” refers to the upstream region of a gene involved in the beginning of transcription of a gene. The two types of shRNA described above also cause the promoter to regulate expression. Here, the expression of the two types of shRNA may be characterized in that they are regulated by two different promoters, respectively. If cloning occurs with identical base sequences using repeated inserts, it is judged highly likely that proper cloning will not occur due to binding between these identical base sequences, resulting in recombination or deletion. The promoters may be RNA polymerase I promoter, RNA polymerase II promoter or RNA polymerase III promoter depending on which RNA polymerase attaches to the promoter and begins transcription. The two promoters above may be characterized in that they are RNA polymerase III promoters (hereinafter pol III promoter). Pol III promoters may be made to transcribe accurately from the 5′ terminal to the 3′ terminal without attaching the cap at the 5′ terminal or the poly (A) tail at the 3′ terminal of the RNA which is transcribed with regulation by the promoter. Types of pol III promoter include, but are not limited to, U6 promoter, H1 promoter and 7SK promoter, etc.

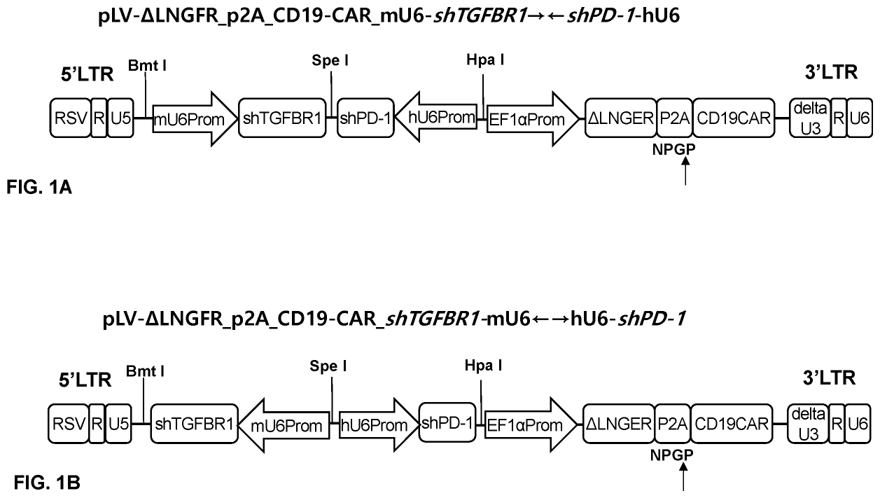

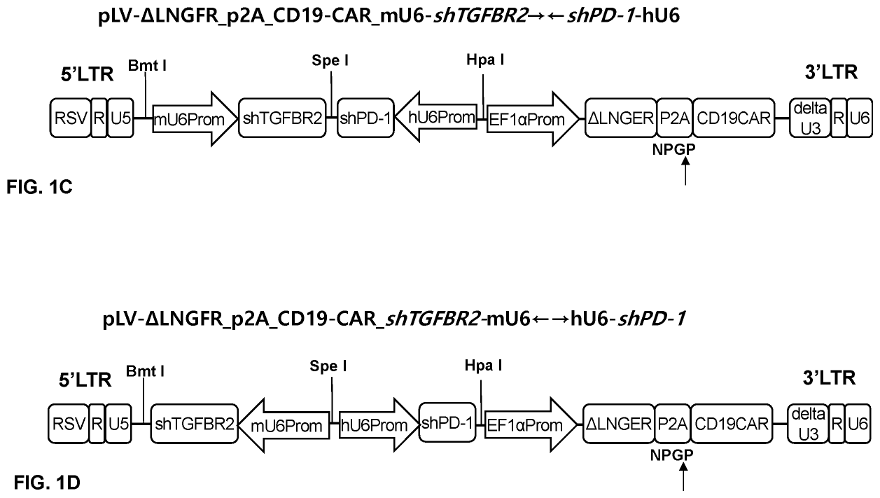

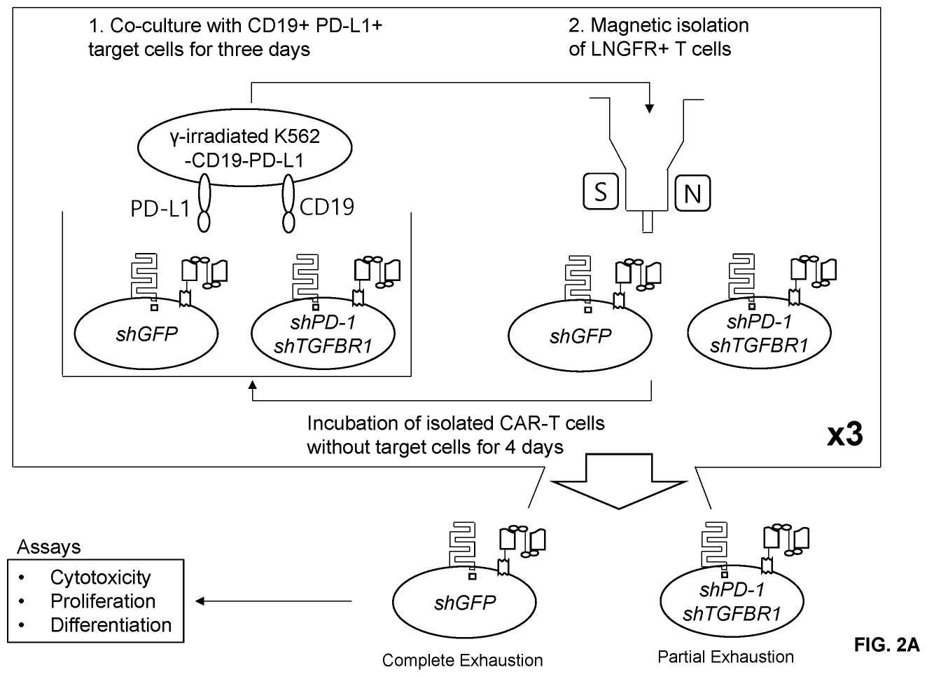

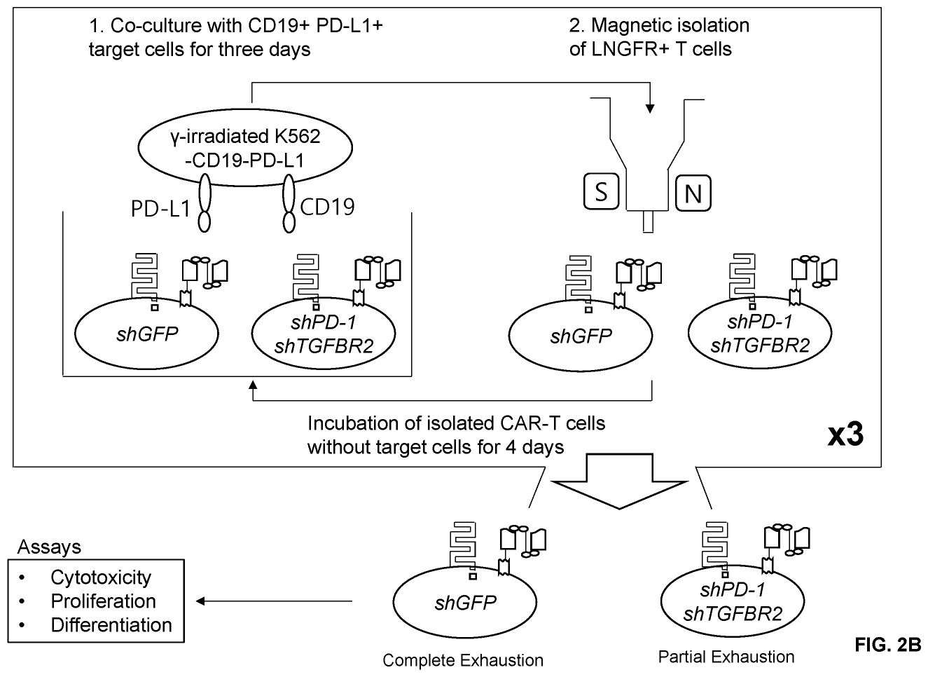

The term “G28z” used herein refers a construct that includes an shGFP expression cassette, a CD28 costimulation domain and a CD3ζ domain (FIG. 1A). More specifically, in the term “G28z”, the “G” represents shGFP; the “28” represents CD28; the “z” represents CD3ζ. Following the same pattern, the term “P28z” used herein refers to a construct that includes an shPD-1 expression cassette, a CD28 costimulation domain, and a CD3 domain (FIG. 1A), wherein “P” represents shPD-1, “28” represents “CD28”, and “z” represents CD3ζ. The term “GBBz” used herein refers to a construct that includes an shGFP expression cassette, a 4-1BB costimulation domain, and a CD3ζ domain (FIG. 1A), wherein the “G” represents shGFP; the “BB” represents 4-1BB; the “z” represents CD3ζ. The term “PBBz” used herein refers to a construct that includes an shPD-1 expression cassette, 4-1BB costimulation domain, and a CD3ζ domain (FIG. 1A), wherein the “P” represents shPD-1; the “BB” represents 4-1BB; the “z” represents CD3ζ.

“CAR” is generally a set of polypeptides which, when existing on an immune cell, causes the immune cell to have specificity to a target cell (normally a cancer cell) while causing signal transduction in the cell. CAR at minimum comprises an extracellular antigen recognition domain which recognizes the target antigen to be described below, a transmembrane domain, and an intracellular signal transduction domain, wherein the intracellular signal transduction domain is derived from the promoting molecules or costimulatory molecules to be described below. The set comprising polypeptides may be attached, or may be in a form where they attach through a switch which is dimerized through stimulation. The promoting molecule may be the zeta chain of the TCR described in the above. “CD19 CAR” is a CAR which targets the CD19 cancer antigen.

The term “T-cell receptor (TCR)” as used herein refers to a protein receptor on T cells that is composed of a heterodimer of an alpha (α) and beta (β) chain, although in some cells the TCR consists of gamma and delta (γ/δ) chains. In certain embodiments, the TCR may be modified on any cell comprising a TCR, including a helper T cell, a cytotoxic T cell, a memory T cell, regulatory T cell, natural killer T cell, and gamma delta T cell, for example.

The term “monoclonal T cell receptor (mTCR)” used herein refers to a T-cell receptor (TCR) that is genetically modified to specifically target a particular antigen. It is can also be referred to as an antigen-specific TCR. T cells having mTCR are reported to be used in immunotherapy, such as adoptive T-cell therapy, for viral infection and cancer. In some aspect, retroviral transfer of chimeric single chain antibody constructs (scFv) has been used as a strategy to produce T cells with defined antigen-specificity. For the most part, chimeric scFv constructs were linked to the intracellular signaling domains of FcR-gamma or CD3 zeta to trigger T-cell effector function. The CD3 zeta domain has been combined with the signaling domains of co-stimulatory molecules such as CD28, 4-1BB or OX40. Monoclonal T cell receptors (mTCRs) and their applications in cancer therapy are described in Stauss et al., 2007, Molecular Therapy, 15(10):1744-50, Zhang and Morgan, 2012, Advanced Drug Delivery Reviews, 64(8): 756-762, and Liddy et al., 2012, Nature Medicine, 18(6):980-7, the content of each of which is herein incorporated by reference in its entirety.

The term “ΔLNGFR” used herein refers to a LNGFR (low-affinity nerve growth factor receptor) without a cytoplasmic domain used for purification of cells wherein the insertion described above has taken place.

An “immune cell” may be characterized herein as selected from, but not limited to, lymphocytes, such as killer T cells, helper T cells, gamma delta T cells and B cells, natural killer cells, mast cells, eosinophils, basophils; and the phagocytic cells include macrophages, neutrophils, and dendritic cells. The T cells include CD4+ T cells and CD8+ T cells.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naïve T cells, regulator T cells, gamma delta T cells (γδ T cells), and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3− and CD56+, expressing at least one of NKG2C and CD57, and optionally, CD16, but lack expression of one or more of the following: PLZF, SYK, FceRγ, and EAT-2. In some embodiments, isolated subpopulations of CD56+ NK cells comprise expression of CD16, NKG2C, CD57, NKG2D, NCR ligands, NKp30, NKp40, NKp46, activating and inhibitory KIRs, NKG2A and/or DNAM-1. CD56+ can be dim or bright expression.

As used herein, the term “immune checkpoints” refer to molecules that exist in the immune system, and are able to turn immune response on or off. Originally, they are safety devices to regulate excessive activation of immune cells, which causes cell death or autoimmune response. These immune checkpoint molecules can be broadly categorized into stimulatory immune checkpoint molecules which increase immune response, and inhibitory immune checkpoint molecules which inhibit immune response. For example, the immune checkpoint receptor and ligands may be selected from a group consisting of PD1 (Programmed cell death protein 1), PD-L1 (Programmed death-ligand 1), CTLA4 (Cytotoxic T-lymphocyte associated protein 4), TIM-3 (T-cell immunoglobulin and mucin-domain containing-3), CEACAM (Carcinoembryonic antigen-related cell adhesion molecule, including the three subtypes CEACAM-1, CEACAM-3 or CEACAM-5), LAG3 (Lymphocyte-activation gene 3), VISTA (V-domain Ig suppressor of T cell activation), BTLA (B- and T-lymphocyte attenuator), TIGIT (T cell immunoreceptor with Ig and ITIM domains), LAIR1 (Leukocyte-associated immunoglobulin-like receptor 1), CD160 (Cluster of differentiation 160), CD96 (Cluster of differentiation 96), MerTK (Proto-oncogene tyrosine-protein kinase MER) and 2B4 (NK cell activation-inducing ligand), and may, for example, be selected between PD1 and TIM3.

The term “culture” or “cell culture refers to the maintenance, growth and/or differentiation of cells in an in vitro environment. “Cell culture media,” “culture media” (singular “medium” in each case), “supplement” and “media supplement” refer to nutritive compositions that cultivate cell cultures. The term “cultivate” or “maintain” refers to the sustaining, propagating (growing) and/or differentiating of cells outside of tissue or the body, for example in a sterile plastic (or coated plastic) cell culture dish or flask. “Cultivation” or “maintaining” may utilize a culture medium as a source of nutrients, hormones and/or other factors helpful to propagate and/or sustain the cells.

A “pharmaceutical composition” for immune therapy in human patients described herein comprises the immune cells. As it is self-evident that, in addition to the cells, other pharmaceutically acceptable salts, carriers, excipients, vehicles and other additives, etc. which may further improve immune response may be added to the pharmaceutical composition, a detailed explanation thereof shall be omitted.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “treating” or “to treat” refer to suppressing, eliminating, reducing, and/or ameliorating a symptom, the severity of the symptom, and/or the frequency of the symptom of the disease being treated. As used herein, the terms “treat,” “treatment” and “treating” also refer to the reduction or amelioration of the progression, severity, and/or duration of a disease or condition resulting from the administration of one or more therapies.

“Effective amount” or “therapeutically effective amount” are used interchangeably herein, and refer to an amount of a compound, formulation, material, or composition, such as T cells, as described herein effective to achieve a particular biological result. Such results may include, but are not limited to, the inhibition of cancer as determined by any means suitable in the art.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

3. Two-in-One Vectors Targeting One or More Immune Checkpoints

Tumor cells express various immune checkpoints, e.g., checkpoint ligands. Therefore, even if one immune checkpoint is inhibited, it might be difficult to expect sustained effect of CAR-T through activation of other immune checkpoints. Combination of monoclonal antibodies has been mainly used to inhibit multiple immune checkpoints and its antitumor effect is been reported continuously (J Clin Invest., 2015, Chauvin J M; PNAS, 2010, Curran M A; Blood, 2018, Wierz M; Cancer cell. 2014, Johnston R J). However, it was known that therapeutic antibodies could induce systemically excessive immune response. In addition, CAR-T cell therapy is also associated with life-threatening cytokine-release syndrome (CRS) and neurotoxicity (Nat Rev Clin Oncol, 2017, Neelapu S S), suggesting that the combination of CAR-T and antibody therapy could maximize the potential of side effects. Furthermore, the conventional concurrent immune cell therapies place an even greater economic burden on patients due to their high cost and that they also act on T cells other than CAR-T and pose a risk of autoimmune symptoms and cytokine release syndrome. The present invention has been devised to address the above problems.

In one embodiment, provided herein is a Two-in-One vector, the vector comprising: a base sequence encoding one or more types of short hairpin RNA (shRNA) which inhibit the expression of genes that weaken the function of immune cells, and a base sequence encoding a chimeric antigen receptor (CAR) a T cell receptor, such as a monoclonal T cell receptor (mTCR).

The vector may be selected from among DNA, RNA, plasmid, lentivirus vector, adenovirus vector and retrovirus vector. For example, lentivirus vector and retrovirus vectorscan insert genes into the genomic DNA of cells allowing for stable expression of the genes. In some embodiments, for example, a Two-in-One lentivirus vector, for example, a dual Two-in-one vector, can be used to genes on the vector into the genome of cells.

In some embodiments, provide is a vector comprising a base sequence encoding two types of short hairpin RNA (shRNA) which inhibit the expression of two different genes that weaken the function of immune cells, and a base sequence encoding any one of a chimeric antigen receptor (CAR) and a T cell receptor, for example a monoclonal T cell receptor (mTCR).

In some embodiments, the expression of the two types of shRNA is characterized in that they are respectively regulated by two different promoters. In some embodiments, the two promoters are RNA polymerase III promoters. In some embodiments, the two promoters are U6 promoters derived from different species. In some embodiments, the two promoters are oriented in different directions from each other on the vector. For example, in a certain embodiment, the promoters are oriented in a head to head orientation. In another embodiment, the promoters are oriented in a tail to tail orientation. In some embodiments, the gene weakening the function of immune cells is an immune checkpoint receptor or ligand.

In some embodiments, the immune checkpoint receptor or ligand is PD1. In some embodiments, the gene weakening the function of immune cells is TGFBR1. In some embodiments, the gene weakening the function of immune cells is TGFBR2.

In some embodiments, the base sequences encoding the first type of shRNA comprises a sequence selected from the group consisting of SEQ ID NOs: 13-23 and the base sequence encoding the second type of shRNA comprises a sequence from the group consisting of SEQ ID NOs: 1-12 In some embodiments, the base sequences encoding the first type of shRNA comprises a sequence selected from the group consisting of SEQ ID NOs: 13-23 and the base sequence encoding the second type of shRNA comprises a sequence from the group consisting of SEQ ID NOs: 43-47.

In some embodiments, the target of the CAR or TCR, for example, mTCR, is a human tumor antigen selected from among increased cancer antigens in cancer or from mutated forms of cancer antigen found in cancer.

In some aspects, provided herein is a vector comprising a sequence selected from the group consisting of SEQ ID NOs: 13-23 and a sequence selected from the group consisting of SEQ ID NOs: 1-12. In other aspects, provided herein is a vector comprising two or more base sequences selected from the group consisting of SEQ ID NOs: 13-23 and two or more base sequences selected from the group consisting of SEQ ID NOs: 1-12. In some aspects, provided herein is a vector comprising a sequence selected from the group consisting of SEQ ID NOs: 13-23 and a sequence selected from the group consisting of SEQ ID NOs: 43-47. In other aspects, provided herein is a vector comprising two or more base sequences selected from the group consisting of SEQ ID NOs: 13-23 and two or more base sequences selected from the group consisting of SEQ ID NOs: 43-47. In some embodiments, the vector is selected from among DNA, RNA, plasmid, lentivirus vector, adenovirus vector and retrovirus vector.

3.1 RNA Interference and Short Hairpin RNA

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2 nucleotide 3′ overhangs and that match the nucleic acid sequence of the target gene. These short RNA species may be naturally produced in vivo by Dicer-mediated cleavage of larger dsRNAs and they are functional in mammalian cells. DNA expression plasmids can be used to stably express the siRNA duplexes or dsRNA described herein in cells and achieve long-term inhibition of the target gene expression. In one aspect, the sense and antisense strands of a siRNA duplex are typically linked by a short spacer sequence leading to the expression of a stem-loop structure termed short hairpin RNA (shRNA). The hairpin is recognized and cleaved by Dicer, thus generating mature siRNA molecules.

Short hairpin RNA (shRNA) as used herein is an RNA molecule wherein some self-complementary sequences create a tight hairpin structure with its stem. The shRNA molecules described herein may be about 40 to 120 nucleotides long, e.g.i, about 70 to 90 nucleotides long. In an exemplary embodiment, the shRNA can be 80 nucleotides long. The shRNA is modeled on micro interfering RNA (miRNA), an endogenous trigger of the RNAi pathway (Lu et al., 2005, Advances in Genetics 54: 117-142, Fewell et al., 2006, Drug Discovery Today 11: 975-982). When shRNA is expressed in a cell, it is processed through a series of steps to become small interfering RNA (siRNA) which acts as a guide for gene silencing Simply put, when shRNA is expressed, it is processed by Drosha complexes in the cell to become pre-shRNA, which is then transported outside the nucleus where it undergoes further processing by a Dicer to become siRNA, and then is single stranded and loaded by an RISC (RNA-induced silencing complex). Here, the antisense strand of siRNA acts as a guide for the RISC complex to attach to the mRNA of the target gene, and gene silencing occurs when the RISC complex, which has attached in this manner, cuts the mRNA. As shRNA in a target gene allows for gene silencing which is lasting and specific to a certain gene, it is included in the vector for the purpose of inhibiting the target gene.

Naturally expressed small RNA molecules, named microRNAs (miRNAs), elicit gene silencing by regulating the expression of mRNAs. The miRNAs containing RISC targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′-UTR of the target mRNAs. A single miRNA may target more than 100 transcripts from various genes, and one mRNA may be targeted by different miRNAs.

siRNA duplexes or dsRNA targeting a specific mRNA may be designed and synthesized in vitro and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

RNAi molecules which were designed to target against a nucleic acid sequence that encodes poly-glutamine repeat proteins which cause poly-glutamine expansion diseases such as Huntington's Disease, are described in U.S. Pat. Nos. 9,169,483 and 9,181,544 and International Patent Publication No. WO2015179525, the content of each of which is herein incorporated by reference in their entirety. U.S. Pat. Nos. 9,169,483 and 9,181,544 and International Patent Publication No. WO2015179525 each provide isolated RNA duplexes comprising a first strand of RNA (e.g., 15 contiguous nucleotides) and second strand of RNA (e.g., complementary to at least 12 contiguous nucleotides of the first strand) where the RNA duplex is about 15 to 30 base pairs in length. The first strand of RNA and second strand of RNA may be operably linked by an RNA loop (˜4 to 50 nucleotides) to form a hairpin structure which may be inserted into an expression cassette. Non-limiting examples of loop portions include SEQ ID NOs: 9-14 of U.S. Pat. No. 9,169,483, the content of which is herein incorporated by reference in its entirety. Non-limiting examples of strands of RNA which may be used, either full sequence or part of the sequence, to form RNA duplexes include SEQ ID NOs: 1-8 of U.S. Pat. No. 9,169,483 and SEQ ID NOs: 1-11, 33-59, 208-210, 213-215 and 218-221 of U.S. Pat. No. 9,181,544, the contents of each of which is herein incorporated by reference in its entirety. Non-limiting examples of RNAi molecules include SEQ ID NOs: 1-8 of U.S. Pat. No. 9,169,483, SEQ ID NOs: 1-11, 33-59, 208-210, 213-215 and 218-221 of U.S. Pat. No. 9,181,544 and SEQ ID NOs: 1, 6, 7, and 35-38 of International Patent Publication No. WO2015179525, the contents of each of which is herein incorporated by reference in their entirety.

In vitro synthetized siRNA molecules may be introduced into cells in order to activate RNAi. An exogenous siRNA duplex, when it is introduced into cells, similar to the endogenous dsRNAs, can be assembled to form the RNA induced silencing complex (RISC), a multiunit complex that interacts with RNA sequences that are complementary to one of the two strands of the siRNA duplex (i.e., the antisense strand). During the process, the sense strand (or passenger strand) of the siRNA is lost from the complex, while the antisense strand (or guide strand) of the siRNA is matched with its complementary RNA. In particular, the targets of siRNA containing RISC complexes are mRNAs presenting a perfect sequence complementarity. Then, siRNA mediated gene silencing occurs by cleaving, releasing and degrading the target.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases, it requires higher concentration of the ss-siRNA to achieve the effective gene silencing potency of the corresponding duplex.

Guidelines for designing siRNAs exist in the art. These guidelines generally recommend generating a 19-nucleotide duplexed region, symmetric 2-3 nucleotide 3′overhangs, 5′-phosphate and 3′-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing mammalian target gene expression may be readily designed.

In one embodiment, a Two-in-One vector includes a base sequence encoding one or more types of short hairpin RNA (shRNA) which inhibit the expression of one or more genes that weaken the function of immune cells, and a base sequence encoding any one of a chimeric antigen receptor (CAR) or a T cell receptor, for example, a monoclonal T cell receptor (mTCR). In another embodiment, a Two-in-One vector comprises a base sequence encoding two types of short hairpin RNA (shRNA) which inhibit the expression of at least two genes that weaken the function of immune cells, and a base sequence encoding any one of a chimeric antigen receptor (CAR) or a T cell receptor, for example, a monoclonal T cell receptor (mTCR).

In some embodiments, the base sequence encodes two types of shRNA, which inhibits the expression of at least two genes that weaken the function of immune cells, wherein the vector can be referred to as a “dual Two-in-One vector.”

In some embodiments, the expression of the two types of shRNA may be characterized in that they are regulated by two different promoters, respectively, to minimize recombination or deletion artifacts during cloning.

In some embodiments, the promoters may be RNA polymerase I promoter, RNA polymerase II promoter or RNA polymerase III promoter depending on which RNA polymerase attaches to the promoter and begins transcription. The two promoters above may be characterized in that they are RNA polymerase III promoters (hereinafter pol III promoters). Pol III promoters may be made to transcribe accurately from the 5′ terminal to the 3′ terminal without attaching the cap at the 5′ terminal or the poly (A) tail at the 3′ terminal of the RNA which is transcribed with regulation by the promoter. Types of pol III promoter include, but are not limited to, U6 promoter, H1 promoter and 7SK promoter, etc. The two promoters included in the vector may be different, selected from among pol III promoters including the three types stated above, and if the same type of promoters are selected, they may be derived from different species. For example, the two promoters may be U6 promoters, e.g., U6 promoters derived from different species, such as U6 promoters derived from humans and mice. As the transcript created by a U6 promoter remains within the nucleus, it is judged that this will be able to cause the Drosha complex that exists in the nucleus to promote the process wherein shRNA is processed into pre-shRNA.

In some embodiments, the two or more promoters may be characterized in that they are oriented in different directions from others on the vector. For example, in a certain embodiment, the promoters are oriented in a head to head (→←) orientation. In another embodiment, the promoters are oriented in a tail to tail (←→) orientation. In a dual Two-in-One vector, to be oriented in different directions on a vector means that when the respective shRNAs whose expression is regulated by the two promoters are transcribed, the directions in which the RNA polymerases move are oriented in different directions on a single nucleic acid molecule. In an exemplary embodiment, the two promoters can be in →← directions (FIGS. 1A and 1C). In another exemplary embodiment, the two promoters can be in ←→ directions (FIGS. 1B and 1D). For example, the two promoters may assume the →← directions on the vector.

In some embodiments the expression of the target genes of the one of more types of shRNA is reduced to about 90% or less of that of a control group, for example the expression of the target genes is reduced to about 80% or less, about 70% or less, about 60% or less, about 50% or less, about 40% or less, about 30% or less, about 20% or less, and about 10% or less of that of a control group.

Ordinarily, shRNA is designed to have a sequence having high homology with part of the mRNA sequence of its target gene (hereinafter the sense shRNA base sequence), a sequence able to produce a sharp hairpin, and a sequence complementary to the sequence having high homology (hereinafter the antisense shRNA base sequence). Non-covalent bonds between the self-complementary portions form a stem structure, and when the shRNA is expressed and processed in the cell, the anti-sense shRNA base sequence acts as a guide for the mRNA of the target gene in the gene silencing process. For example, the base sequences of a cassette used in herein for the expression of shRNA can comprise a NNNNNNNNNNNNNNNNNNNNN (21 base)—loop sequence—NNNNNNNNNNNNNNNNNNNN (19 base) structure. In one embodiment, the 21 base stretch encodes the sense shRNA base sequences and the 19 base stretch is complementary or substantially complementary to the 21 base stretch and encodes the anti-sense shRNA base sequences. In another embodiment, the 19 base stretch encodes the sense shRNA base sequences and the 21 base stretch is complementary or substantially complementary to the 19 base stretch and encodes the anti-sense shRNA base sequences. When expressed, therefore, the resulting RNA forms a stem and loop structure. In certain embodiments, two such sense or anti-sense shRNA base sequences for each target gene (human derived) which may be included in the cassette are selected from a group consisting of SEQ ID NOs: 13-23 and from the group consisting of SEQ ID NOs: 1-12. In specific embodiments, the base sequences of the cassette used herein comprise shRNA sequences targeting PD-1 and TGFBR2. In certain embodiments, two such sense or anti-sense shRNA base sequences for each target gene (human derived) which may be included in the cassette are selected from a group consisting of SEQ ID NOs: 13-23 and from the group consisting of SEQ ID NOs: 43-47. In specific embodiments, the base sequences of the cassette used herein comprise shRNA sequences targeting PD-1 and TGFBR1.

In some embodiments, the entire shRNA base sequence can be positioned at the 3′ terminal of a mouse or human U6 promoter, and the TTTTT necessary for terminating transcription by the U6 promoter can be positioned at the 3′ terminals of all of the shRNA base sequences.

In some embodiments, the nucleic acid sequences of the respective shRNAs may, in addition to the sequences described herein, comprise nucleic acid sequences exhibiting at least 50%, specifically at least 70%, more specifically at least 80%, even more specifically at least 90%, and most specifically at least 95% sequence homology with these sequences. This is because, in the case of siRNA (small interfering RNA) and shRNA which is processed intracellularly to become siRNA in particular, it has been reported that some degree of mutation, especially mutation at the 5′ terminal is tolerable, causing normal knockdown of the target gene, and that mutations of siRNA and shRNA made to have a structure similar to that of miRNA that plays a role in gene silencing more effectively induce knockdown of the target gene. Further, in the use of vectors, self-evident to those skilled in the art are variations within the vector, that is, the addition, modification or deletion of base sequences which may occur in the cloning process for introducing a certain sequence into the vector, or changes to or introduction of components to improve the ease of use of the vector of the degree to which the intended gene is expressed.

Various modes of action exist for genes which weaken the function of immune cells. Examples include inhibiting proliferation of immune cells or causing cell death, reducing reactions with molecules with which immune cells need to react with in order to become activated, inhibiting the expression of genes necessary for immune cells to recognize reaction targets, and causing differentiation into different types of immune cell to play a different function instead of causing immune response to a particular target. Representative examples include, but are not limited to, molecules associated with the immune checkpoints to be explained below.

In some embodiments, the gene weakening the function of immune cells are PD-1, TGFBR1 and/or TGFBR2.

3.2 Chimeric Antigen Receptor (CAR) and T Cell Receptor, for Example, Monoclonal T Cell Receptor (mTCR).

In one embodiment, provided herein is a Two-in-One vector includes a base sequence encoding one or more types of short hairpin RNA (shRNA) which inhibit the expression of one or more genes that weaken the function of immune cells, and a base sequence encoding any one of a chimeric antigen receptor (CAR) or a T cell receptor, for example, a monoclonal T cell receptor (mTCR). In another embodiment, provided herein is a Two-in-One vector that comprises a base sequence encoding two types of short hairpin RNA (shRNA) which inhibit the expression of at least two genes that weaken the function of immune cells, and a base sequence encoding any one of a chimeric antigen receptor (CAR) or a T cell receptor, for example, a monoclonal T cell receptor (mTCR).

CAR is generally a set of polypeptides which, when existing on an immune cell, causes the immune cell to have specificity to a target cell (normally a cancer cell) while causing signal transduction in the cell. CAR at minimum comprises an extracellular antigen recognition domain which recognizes the target antigen to be described below, a transmembrane domain, and an intracellular signal transduction domain, wherein the intracellular signal transduction domain is derived from the promoting molecules or costimulatory molecules.

The structure of CARs commonly used today for clinical applications comprises a single chain variable fragment domain (hereinafter scFv) which gives specificity to an antigen, a spacer domain to regulate the distance between the scFv and the cell membrane, a transmembrane domain, and an intracellular signaling domain (hereinafter ISD). The ISD in turn comprises a costimulatory domain (CD28, CD137 or OX40) which contributes to in vivo proliferation and long life of one or multiple T cells, and a TCR signaling domain (CD3 zeta, CD3ζ) which contributes to T cell activation. T-cells modified to express CAR that have been prepared in this manner can be activated by recognizing cancer cells which express the target antigen with high specificity, effectively induce the death of such cancer cells, simultaneously proliferate exponentially in the body, and remain alive for a long time. For example, when CAR-T cells (CART-19) prepared to target CD19, a B cell-specific antigen, were administered to a B-cell leukemia patient, it was reported that the cells proliferated to 1,000 to 10,000 times and remained alive in the body for several years. As a result, CART-19 exhibited 90% complete response in a clinical trial carried out on terminal acute lymphoblastic leukemia (B-ALL) patients on whom conventional chemotherapy, etc., had not been effective, leading to a rare case of licensing to a global pharmaceuticals company in the early investigator-initiated clinical trial phase. It became the first CAR-T cell therapy agent to receive U.S. FDA approval in 2017, and thereafter, a second CAR-T was also approved.

On the surface of immune cells, for example T cells, exist immune checkpoint receptors such as CTLA-4 (cytotoxic T-lymphocyte associated protein-4) or PD-1 (programmed cell death protein-1). These receptors are originally safety devices to regulate excessive activation and cell death of T cells, or the triggering of autoimmune responses. However, cancer cells, especially solid cancers, are reported to use this to avoid immunosurveillance by T cells. For example, if a cancer cell expresses PD-L1 (programmed death-ligand 1) on the surface, a T cell which expresses PD-1, the receptor therefore, recognizes the cancer cell and is activated, but will soon become exhausted by an activation inhibition signal from the PD-1. To prevent inhibition of T cell activity by signals from these immune checkpoint receptors, monoclonal antibodies to CTLA4 or PD-1, etc. that inhibit signal transmittance by target immune checkpoint receptors were developed. Therapies which improve the overall immune function of T cells through the blocking of immune checkpoints by using these immune checkpoint receptor inhibitors are exhibiting efficacy against various solid cancers as well.

As CAR-T cells are also ultimately a therapy that relies on the cytotoxicity of activated T cells, the existence of an immunosuppressive environment around CAR-T cells acts as a major hindrance to their therapeutic effect. In fact, unlike their therapeutic effects exhibited in B-cell leukemia, CAR-Ts prepared to target solid tumors have rarely exhibited hopeful therapeutic effects. This is thought to be because solid tumors, unlike blood cancers, create immune-suppressive tumor microenvironments to suppress the activity and proliferation of CAR-T cells. Further, even among B cell blood cancers, it has been reported that unlike acute lymphoblastic leukemia (ALL) patients of whom almost 90% were responsive to therapy using CART-19, the therapeutic effects were relatively less in lymphoma patients (20 to 50% response) or chronic lymphoblastic leukemia patients (CLL, around 20% response).

Further, it was reported that PD-L1 and other immunosuppressive ligands are expressed in the tumor microenvironments formed by lymphoma, according to which the function of T cells within cancerous tissue is exhausted. Further, it has been reported that the T cells obtained from CLL patients had already been substantially exhausted, with high degrees of expression of immune checkpoint receptors such as PD-1, CD160 and CD244.

Pre-clinical trial results showing that simultaneous use of anti-CTLA or anti-PD-1 inhibitory antibodies with CAR-T cells to recover this lowered activity of CAR-T cells improves the anti-cancer effect were reported, and clinical trials using these combinations are currently underway. However, a problem with such concurrent therapies of antibody and CAR-T cells is that the antibodies spread out throughout the body impact not just the CAR-T cells but all other T cells that exist in the body, potentially resulting in severe and systemic adverse reactions such as cytokine release syndrome, as well as autoimmune symptoms. Another problem which has been pointed out is the increased cost of treatment resulting from concurrent use of expensive antibody therapies with cell therapy.

Accordingly, there have recently been attempts to regulate gene expression within cells to allow for suppression of the immune checkpoints of CAR-T cells. International patent application publication WO2016/069282 discloses compositions and methods for generating a modified T cell with a nucleic acid capable of downregulating endogenous gene expression selected from the group consisting of TCR α chain, TCR β chain, β-2 microglobulin and FAS further comprising a nucleic acid encoding a modified T cell receptor (TCR) comprising affinity for a surface antigen on a target cell or an electroporated nucleic acid encoding a chimeric antigen receptor (CAR). The publication states that gene scissors such as CRISPR/Cas9 can be used to knock-out the expression of endogenous genes, but the method of preparation of the CAR-T cells disclosed in the patent publication is rather complicated, and has the problems of low production yield and high production cost.

Meanwhile, international patent publication WO2015/090230 discloses that one single type of short hairpin RNA (shRNA) inhibiting a molecule that additionally suppresses the function of T cells, may be used on cells expressing CAR. As the cost burden of cell therapy is high, a patient faces various burdens in the event of failure. A single type of shRNA, however, may not be able to effectively inhibit the activity of such target molecules. In some embodiments, the set comprising polypeptides may be attached. In some embodiments, the set comprising polypeptide may be in a form where they attach through a switch which is dimerized through stimulation. In some embodiments, the CAR may be a fusion protein which comprises the extracellular antigen recognition domain, the transmembrane domain and the intracellular signal transduction domain. In further embodiments, the CAR fusion protein may additionally comprise a leader sequence at the N terminal, and the leader sequence may be cut away in the process of the CAR being expressed and becoming anchored in the cell membrane.

The TCR, for example, mTCR, described herein may comprise a chain selected from among α, β, γ and δ chains. In some embodiments, the chains are able to recognize the target antigen to be described below, CD3, and a zeta chain, and additionally a costimulatory molecule, where the costimulatory molecule may be selected from among ICOS, OX40, CD137 (4-1BB), CD27 or CD28.

In some embodiments, retroviral transfer of chimeric single chain antibody constructs (scFv) can be used to produce TCR, for example mTCR with defined antigen-specificity. In further embodiments, chimeric scFv constructs can be linked to the intracellular signaling domains of FcR-gamma or CD3ζ to trigger T-cell effector function. In the CD3ζ domain can be combined with the signaling domains of costimulatory molecules, wherein antibody engagement can trigger effector T-cell function and also deliver co-stimulatory signals. In further embodiments, the costimulatory molecules can be selected from CD28, 4-1BB and OX40.

In some embodiments, the Two-in-One vectors can comprise a CD3ζ domain, wherein the chains of the TCR, for example, mTCR can form a complex through noncovalent bonds with CD3 and a zeta (ζ) chain. When an antigen is recognized through the antigen recognition sites of the chains, the CD3 and zeta chains send signals into the cytoplasm of immune cells on which such TCR complex is expressed, inducing functional activation.

In some embodiments, the intracellular signal transduction domain may additionally comprise one or more functional signal transduction domains derived from the costimulatory molecules. In some embodiments, the promoting molecule may be the zeta chain of the TCR described in the above.

CAR and TCR, for example, mTCR are cell surface receptors. In some embodiments, the target of the CAR or TCR, for example, mTCR may be characterized in that it is a cancer antigen whose expression is specifically increased in cancer. In some embodiments, the target of the CAR or TCR, for example, mTCR may be characterized in that it is a cancer antigen that exists in mutated forms in cancer.

In some embodiments, the target of the CAR or TCR, for example, mTCR may be a human tumor antigen whose expression is increased in a cancer which is to be treated. For example, the target can be selected from 5T4 (trophoblast glycoprotein), 707-AP, 9D7, AFP (α-fetoprotein), AlbZIP (androgen-induced bZIP), HPG1 (human prostate specific gene-1), α5β1-Integrin, α5β6-Integrin, α-methylacyl-coenzyme A racemase, ART-4 (ADPribosyltransferase-4), B7H4 (v-set domain-containing T-cell activation inhibitor 1), BAGE-1 (B melanoma antigen-1), BCL-2 (B-cell CLL/lymphoma-2), BING-4 (WD repeat domain 46), CA 15-3/CA 27-29 (mucin 1), CA 19-9 (cancer antigen 19-9), CA 72-4 (cancer antigen 72-4), CA125 (cancer antigen 125), calreticulin, CAMEL (CTL-recognized antigen on melanoma), CASP-8 (caspase 8), cathepsin B, cathepsin L, CD19 (cluster of differentiation 19), CD20, CD22, CD25, CD30, CD33, CD4, CD52, CD55, CD56, CD80, CEA (carcinoembryonic antigen SG8), CLCA2 (chloride channel accessory 2), CML28 (chronic myelogenous leukemia tumor antigen 28), Coactosin-like protein, Collagen XXIII, COX-2 (cyclooxygenase-2), CT-9/BRD6 (cancer/testis antigen 9), Cten (c-terminal tensin-like protein), cyclin B1, cyclin D1, cyp-B, CYPB1 (cytochrome p450 family 1 subfamily b member 1), DAM-10/MAGE-B1 (melanoma-associated antigen B1), DAM-6/MAGE-B2, EGFR/Her1 (epidermal growth factor receptor), EMMPRIN (basigin), EpCam, EphA2 (EPH receptor A2), EphA3, ErbB3 (Erb-B2 receptor tyrosine kinase 3), EZH2 (enhancer of zeste 2 polycomb repressive complex 2 subunit), FGF-5 (fibroblast growth factor 5), FN (fibronectin), Fra-1 (Fosrelated antigen-1), G250/CAIX (carbonic anhydrase 9), GAGE-1 (G antigen-1), GAGE-2, GAGE-3, GAGE-4, GAGE-5, GAGE-6, GAGE-7b, GAGE-8, GDEP (gene differentially expressed in prostate), GnT-V (gluconate kinase), gp100 (melanocytes lineage-specific antigen GP100), GPC3 (glypican3), HAGE (helical antigen), HAST-2 (sulfotransferase family 1A member 1), hepsin, Her2/neu/ErbB2 (Erb-B2 receptor tyrosine kinase 2), HERV-K-MEL, HNE (medullasin), homeobox NKX 3.1, HOM-TES-14/SCP-1, HOM-TES-85, HPV-E6, HPVE7, HST-2 (sirtuin-2), hTERT, iCE (caspase 1), IGF-1R (insulin like growth factor-1 receptor), IL-13Ra2 (interleukin-13 receptor subunit α2), IL-2R (interleukin-2 receptor), IL-5 (interleukin-5), immature laminin receptor, kallikrein 2, kallikrein 4, Ki67, KIAA0205 (lysophosphatidylglycerol acyltransferase 1), KK-LC-1 (kita-kyushu lung cancer antigen-1), KM-HN-1, LAGE-1 (L antigen family member-1), Livin, MAGE-A1, MAGE-A10, MAGE-Al2, MAGEA2, MAGE-A3, MAGE-A4, MAGE-A6, MAGE-A9, MAGE-B1, MAGE-B10, MAGE-B16, MAGEB17, MAGE-B2, MAGE-B3, MAGE-B4, MAGE-B5, MAGE-B6, MAGE-C1, MAGE-C2, MAGE-C3, MAGE-D1, MAGE-D2, MAGE-D4, MAGE-E1, MAGE-E2, MAGE-F1, MAGE-H1, MAGEL2 (melanoma antigen family L2), mammaglobin A, MART-1/Melan-A (melanoma antigen recognized by T-cells-1), MART-2, matrix protein 22, MC1R (melanocortin 1 receptor), M-CSF (macrophage colony-stimulating factor), Mesothelin, MG50/PXDN (peroxidasin), MMP 11 (matrix metalloprotease 11), MN/CA IX-antigen (carbonic anhydrase 9), MRP-3 (multidrug resistance-associated protein-3), MUC1 (mucin 1), MUC2, NA88-A (VENT-like homeobox 2 pseudogene 1), N-acetylglucos-aminyltransferase-V, Neo-PAP (Neo-poly (A) polymerase), NGEP (new gene expressed in prostate), NMP22 (nuclear matrix protein 22), NPM/ALK (nucleophosmin), NSE (neuron-specific enolase), NY-ESO-1, NY-ESO-B, OA1 (osteoarthritis QTL 1), OFA-iLRP (oncofetal antigen immature laminin receptor protein), OGT (O-GlcNAc transferase), OS-9 (endoplasmic reticulum lectin), osteocalcin, osteopontin, p15 (CDK inhibitor 2B), p53, PAGE-4 (P antigen family member-4), PAI-1 (plasminogen activator inhibitor-1), PAI-2, PAP (prostatic acid phosphatase), PART-1 (prostate androgen-regulated transcript 1), PATE (prostate and testis expressed 1), PDEF (prostate-derived Ets factor), Pim-l-Kinase (proviral integration site 1), Pin1 (Peptidyl-prolyl cis-trans isomerase NIMA-interacting 1), POTE (expressed in prostate, ovary, testis, and placenta), PRAME (preferentially expressed antigen in melanoma), prostein, proteinase-3, PSA (prostate-specific antigen), PSCA (prostate stem cell antigen), PSGR (prostate-specific G-protein coupled receptor), PSM, PSMA (prostate specific membrane antigen), RAGE-1 (renal tumor carcinoma antigen), RHAMM/CD168, RU1 (renal ubiquitous protein 1), RU2, SAGE (sarcoma antigen), SART-1 (squamous cell carcinoma antigen recognized by T-cells-1), SART-2, SART-3, Sp17 (sperm protein 17), SSX-1 (SSX family member 1), SSX-2/HOM-MEL-40, SSX-4, STAMP-1 (STEAP2 metalloreductase), STEAP, survivin, survivin-213, TA-90 (tumor associated antigen-90), TAG-72 (tumor associated glycoprotein-72), TARP (TCRγ alternate reading frame protein), TGFb (transforming growth factor β), TGFbR11 (transforming growth factor β receptor 11), TGM-4 (transglutaminase 4), TRAG-3 (taxol resistance associated gene 3), TRG (T-cell receptor γ locus), TRP-1 (transient receptor potential-1), TRP-2/6b, TRP-2/INT2, Trp-p8, Tyrosinase, UPA (U-plasminogen activator), VEGF (vascular endothelial growth factor A), VEGFR-2/FLK-1 and WT1 (wilms tumor 1), or may be a mutated form of human tumor antigen discovered in the cancer to be treated, selected from among α-actinin-4/m, ARTC1/m, bcr/abl, beta-Catenin/m, BRCA1/m, BRCA2/m, CASP-5/m, CASP-8/m, CDC27/m, CDK4/m, CDKN2A/m, CML66, COA-1/m, DEK-CAN, EFTUD2/m, ELF2/m, ETV6-AML1, FN1/m, GPNMB/m, HLA-A*0201-R170I, HLA-A11/m, HLA-A2/m, HSP70-2M, KIAA0205/m, K-Ras/m, LDLR-FUT, MART2/m, ME1/m, MUM-1/m, MUM-2/m, MUM-3/m, Myosin class 1/m, neo-PAP/m, NFYC/m, N-Ras/m, OGT/m, OS-9/m, p53/m, Pm1/RARa, PRDX5/m, PTPRX/m, RBAF600/m, SIRT2/m, SYTSSX-1, SYT-SSX-2, TEL-AML1, TGFbRII and TPI/m. For example, the target antigen may be selected between CD19 or CD22.

3.3 Components of the Two-in-One Vectors

As provided herein, a Two-in-One vector includes a base sequence encoding one or more types of short hairpin RNA (shRNA) which inhibit the expression of one or more genes that weaken the function of immune cells, and a base sequence encoding a chimeric antigen receptor (CAR) or a T cell receptor (TCR), for example, monoclonal T cell receptor (mTCR).

In some embodiments, the Two-in-One vectors can comprise sequences encoding factors that promote the insertion of the sequences into the host cell genome. In some embodiments, the sequences are located at either or both end(s) of the vector gene. In some embodiments, the sequences are LTRs (long terminal sequence).

In some embodiments, the Two-in-One vectors can comprise a domain encodes proteins that is used for purification of cells wherein the insertion described above has taken place. In some embodiments, the domain is a ΔLNGFR domain, wherein ΔLNGFR is a LNGFR (low-affinity nerve growth factor receptor) without a cytoplasmic domain used for purification of cells wherein the insertion described above has taken place.

In some embodiments, the Two-in-One vectors can comprise promoters that induce the expression of both ΔLNGFR and CAR characterized by sustained expression, for example an EF 1 a promoter inducing expression of both ΔLNGFR and CD19 CAR. In further embodiments, ΔLNGFR and CD19 CAR are first transcribed in a form wherein they exist on a single mRNA. In such embodiments where two or more cistrons exist on the same mRNA, an IRES (internal ribosome entry site) may be inserted there between to cause expression of both cistrons. However, IRES is excessively long, and it has been reported that the expression efficiency of the downstream cistron is reduced. In some embodiments, components other than IRES can be used to overcome such disadvantages, for example a P2A (2a peptide), wherein, during translation, the ribosome passes without forming a peptide bond at the C terminal of P2A, allowing for the downstream gene to be expressed later.

In some embodiments, the base sequences included in the vector, and the nucleic acid sequences of the respective shRNAs may, in addition to the sequences described herein, comprise nucleic acid sequences exhibiting at least 50%, specifically at least 70%, more specifically at least 80%, even more specifically at least 90%, and most specifically at least 95% sequence homology with these sequences. This is because, in the case of siRNA (small interfering RNA) and shRNA which is processed intracellularly to become siRNA in particular, it has been reported that some degree of mutation, especially mutation at the 5′ terminal is tolerable, causing normal knockdown of the target gene, and that mutations of siRNA and shRNA made to have a structure similar to that of miRNA that plays a role in gene silencing more effectively induce knockdown of the target gene. Further, in the use of vectors, self-evident to those skilled in the art are variations within the vector, that is, the addition, modification or deletion of base sequences which may occur in the cloning process for introducing a certain sequence into the vector, or changes to or introduction of components to improve the ease of use of the vector of the degree to which the intended gene is expressed.

4. Production and Evaluation of CAR-T Cells that Targeting One or More Immune Checkpoints

As provided herein, a Two-in-One vector includes a base sequence encoding one or more types of short hairpin RNA (shRNA) which inhibit the expression of genes that weaken the function of immune cells, and a base sequence encoding any one of a chimeric antigen receptor (CAR) and a T cell receptor (TCR), for example, monoclonal T cell receptor (mTCR). The vector can be used for the production of immune cells having inhibited expression of genes that weaken the function of immune cell. In some embodiments, the vector is selected from the group consisting of DNA, RNA, plasmid, lentivirus vector, adenovirus vector, and retrovirus vector.

In one aspect, the immune cell described herein is characterized in that it comprises the above vector and expresses CAR or a TCR, for example, mTCR, and in that expression of the target genes of the one or more types of shRNA is reduced. In some embodiments the expression of the target genes of the one of more types of shRNA is reduced to about 90% or less of that of a control group, for example the expression of the target genes is reduced to about 80% or less, about 70% or less, about 60% or less, about 50% or less, about 40% or less, about 30% or less, about 20% or less, and about 10% or less of that of a control group. In some embodiments, the immune cell is selected from between human-derived T cells and NK cells.

In another aspect, provided herein are methods. In some embodiments, provided is a method of producing an immune cell comprising introducing into an immune cell, simultaneously or sequentially in any order: (1) a gene encoding a genetically engineered antigen receptor that specifically binds to a target antigen; and (2) a genetic disruption agent reducing or capable of reducing expression in the immune cell of at least two genes that weaken the function of the immune cell, thereby producing an immune cell in which a genetically engineered antigen receptor is expressed and expression of the genes that weaken the function of the immune cell is reduced.

In some embodiments, the genetically engineered antigen receptor is a chimeric antigen receptor (CAR) or a T cell receptor (TCR). In some embodiments, the genetically engineered antigen receptor is a CAR. In some embodiments, the CAR comprises an extracellular antigen recognition domain, a transmembrane domain, and an intracellular signal transduction domain.

In some embodiments, the extracellular antigen recognition domain of the CAR specifically binds to the target antigen.

In some embodiments, the intracellular signal transduction domain of the CAR comprises an intracellular domain of a CD3 zeta (CD3ζ) chain. In some embodiments, the intracellular signal transduction domain of the CAR further comprises a costimulatory molecule.

In some embodiments, the costimulatory molecule is selected from the group consisting of ICOS, OX40, CD137 (4-1BB), CD27, and CD28. In some embodiments, the costimulatory molecule is CD137 (4-1BB). In some embodiments, the costimulatory molecule is CD28.

In some embodiments, the target antigen is expressed on the cell surface of a cancer cell, a cancer tissue, and/or a tumor microenvironment. In some embodiments, the target antigen is either a cancer antigen whose expression is increased, or a mutated form of a cancer antigen, in the cancer cell, the cancer tissue, and/or the tumor microenvironment.

In some embodiments, the cancer antigen whose expression is increased in the cancer cell, the cancer tissue, and/or the tumor microenvironment is selected from the group consisting of: 5T4 (trophoblast glycoprotein), 707-AP, 9D7, AFP (α-fetoprotein), AlbZIP (androgen-induced bZIP), HPG1 (human prostate specific gene-1), α5β1-Integrin, α5β6-Integrin, α-methylacyl-coenzyme A racemase, ART-4 (ADPribosyltransferase-4), B7H4 (v-set domain-containing T-cell activation inhibitor 1), BAGE-1 (B melanoma antigen-1), BCL-2 (B-cell CLL/lymphoma-2), BING-4 (WD repeat domain 46), CA 15-3/CA 27-29 (mucin 1), CA 19-9 (cancer antigen 19-9), CA 72-4 (cancer antigen 72-4), CA125 (cancer antigen 125), calreticulin, CAMEL (CTL-recognized antigen on melanoma), CASP-8 (caspase 8), cathepsin B, cathepsin L, CD19 (cluster of differentiation 19), CD20, CD22, CD25, CD30, CD33, CD4, CD52, CD55, CD56, CD80, CEA (carcinoembryonic antigen SG8), CLCA2 (chloride channel accessory 2), CML28 (chronic myelogenous leukemia tumor antigen 28), Coactosin-like protein, Collagen XXIII, COX-2 (cyclooxygenase-2), CT-9/BRD6 (cancer/testis antigen 9), Cten (c-terminal tensin-like protein), cyclin B1, cyclin D1, cyp-B, CYPB1 (cytochrome p450 family 1 subfamily b member 1), DAM-10/MAGE-B1 (melanoma-associated antigen B1), DAM-6/MAGE-B2, EGFR/Her1 (epidermal growth factor receptor), EMMPRIN (basigin), EpCam, EphA2 (EPH receptor A2), EphA3, ErbB3 (Erb-B2 receptor tyrosine kinase 3), EZH2 (enhancer of zeste 2 polycomb repressive complex 2 subunit), FGF-5 (fibroblast growth factor 5), FN (fibronectin), Fra-1 (Fosrelated antigen-1), G250/CAIX (carbonic anhydrase 9), GAGE-1 (G antigen-1), GAGE-2, GAGE-3, GAGE-4, GAGE-5, GAGE-6, GAGE-7b, GAGE-8, GDEP (gene differentially expressed in prostate), GnT-V (gluconate kinase), gp100 (melanocytes lineage-specific antigen GP100), GPC3 (glypican3), HAGE (helical antigen), HAST-2 (sulfotransferase family 1A member 1), hepsin, Her2/neu/ErbB2 (Erb-B2 receptor tyrosine kinase 2), HERV-K-MEL, HNE (medullasin), homeobox NKX 3.1, HOM-TES-14/SCP-1, HOM-TES-85, HPV-E6, HPVE7, HST-2 (sirtuin-2), hTERT, iCE (caspase 1), IGF-1R (insulin like growth factor-1 receptor), IL-13Ra2 (interleukin-13 receptor subunit α2), IL-2R (interleukin-2 receptor), IL-5 (interleukin-5), immature laminin receptor, kallikrein 2, kallikrein 4, Ki67, KIAA0205 (lysophosphatidylglycerol acyltransferase 1), KK-LC-1 (kita-kyushu lung cancer antigen-1), KM-HN-1, LAGE-1 (L antigen family member-1), Livin, MAGE-A1, MAGE-A10, MAGE-A12, MAGEA2, MAGE-A3, MAGE-A4, MAGE-A6, MAGE-A9, MAGE-B1, MAGE-B10, MAGE-B16, MAGEB17, MAGE-B2, MAGE-B3, MAGE-B4, MAGE-B5, MAGE-B6, MAGE-C1, MAGE-C2, MAGE-C3, MAGE-D1, MAGE-D2, MAGE-D4, MAGE-E1, MAGE-E2, MAGE-F1, MAGE-H1, MAGEL2 (melanoma antigen family L2), mammaglobin A, MART-1/Melan-A (melanoma antigen recognized by T-cells-1), MART-2, matrix protein 22, MC1R (melanocortin 1 receptor), M-CSF (macrophage colony-stimulating factor), Mesothelin, MG50/PXDN (peroxidasin), MMP 11 (matrix metalloprotease 11), MN/CA IX-antigen (carbonic anhydrase 9), MRP-3 (multidrug resistance-associated protein-3), MUC1 (mucin 1), MUC2, NA88-A (VENT-like homeobox 2 pseudogene 1), N-acetylglucos-aminyltransferase-V, Neo-PAP (Neo-poly (A) polymerase), NGEP (new gene expressed in prostate), NMP22 (nuclear matrix protein 22), NPM/ALK (nucleophosmin), NSE (neuron-specific enolase), NY-ESO-1, NY-ESO-B, OA1 (osteoarthritis QTL 1), OFA-iLRP (oncofetal antigen immature laminin receptor protein), OGT (O-GlcNAc transferase), OS-9 (endoplasmic reticulum lectin), osteocalcin, osteopontin, p15 (CDK inhibitor 2B), p53, PAGE-4 (P antigen family member-4), PAI-1 (plasminogen activator inhibitor-1), PAI-2, PAP (prostatic acid phosphatase), PART-1 (prostate androgen-regulated transcript 1), PATE (prostate and testis expressed 1), PDEF (prostate-derived Ets factor), Pim-1-Kinase (proviral integration site 1), Pin1 (Peptidyl-prolyl cis-trans isomerase NIMA-interacting 1), POTE (expressed in prostate, ovary, testis, and placenta), PRAME (preferentially expressed antigen in melanoma), prostein, proteinase-3, PSA(prostate-specific antigen), PSCA (prostate stem cell antigen), PSGR (prostate-specific G-protein coupled receptor), PSM, PSMA (prostate specific membrane antigen), RAGE-1 (renal tumor carcinoma antigen), RHAMM/CD168, RU1 (renal ubiquitous protein 1), RU2, SAGE (sarcoma antigen), SART-1 (squamous cell carcinoma antigen recognized by T-cells-1), SART-2, SART-3, Sp17 (sperm protein 17), SSX-1 (SSX family member 1), SSX-2/HOM-MEL-40, SSX-4, STAMP-1 (STEAP2 metalloreductase), STEAP, survivin, survivin-213, TA-90 (tumor associated antigen-90), TAG-72 (tumor associated glycoprotein-72), TARP (TCRγ alternate reading frame protein), TGFb (transforming growth factor β), TGFbR11 (transforming growth factor β receptor 11), TGM-4 (transglutaminase 4), TRAG-3 (taxol resistance associated gene 3), TRG (T-cell receptor γ locus), TRP-1 (transient receptor potential-1), TRP-2/6b, TRP-2/INT2, Trp-p8, Tyrosinase, UPA (U-plasminogen activator), VEGF (vascular endothelial growth factor A), VEGFR-2/FLK-1, and WT1 (wilms tumor 1).

In some embodiments, the target antigen is CD19 or CD22. In some embodiments, the target antigen is CD19. In some embodiments, the target is EGFR. In exemplary embodiments, the CAR is derived from cetuximab. In some embodiments, the CAR is a single chain Fv (scFv). In specific embodiments, the scFv has the sequence of SEQ ID NO: 62.

In some embodiments, the mutated form of a tumor antigen is selected from the group consisting of: α-actinin-4/m, ARTC1/m, bcr/abl, beta-Catenin/m, BRCA1/m, BRCA2/m, CASP-5/m, CASP-8/m, CDC27/m, CDK4/m, CDKN2A/m, CML66, COA-1/m, DEK-CAN, EFTUD2/m, ELF2/m, ETV6-AML1, FN1/m, GPNMB/m, HLA-A*0201-R170I, HLA-A11/m, HLA-A2/m, HSP70-2M, KIAA0205/m, K-Ras/m, LDLR-FUT, MART2/m, ME1/m, MUM-1/m, MUM-2/m, MUM-3/m, Myosin class 1/m, neo-PAP/m, NFYC/m, N-Ras/m, OGT/m, OS-9/m, p53/m, Pml/RARa, PRDX5/m, PTPRX/m, RBAF600/m, SIRT2/m, SYTSSX-1, SYT-SSX-2, TEL-AML1, TGFbRII, and TPI/m.

In some embodiments, expression of the genes that weaken the function of the immune cell causes one or more of the following: i) inhibits proliferation of the immune cell; ii) induces cell death of the immune cell; iii) inhibits the function of a molecule necessary for the immune cell to recognize the target antigen and/or to get activated; iv) induces differentiation of the immune cell into a different type that plays a different function instead of causing immune response to the target antigen; v) decreases reactions of the immune cell with a molecule which promotes immune response of the immune cell; or vi) increases reactions of the immune cell with a molecule which suppresses immune response of the immune cell.

In some embodiments, the gene that weakens the function of the immune cell is PD1, TGFBR1, or TGFBR2. In some embodiments, the genes that weaken the function of the immune cell increases reactions of the immune cell with a molecule which suppresses immune response of the immune cell. In some embodiments, the gene that increases reactions of the immune cell with a molecule which suppresses immune response of the immune cell encodes an immune checkpoint receptor or ligand.

In some embodiments, the immune checkpoint receptor or ligand is PD1, In some embodiments, the genetic disruption agent reduces the expression of at least two genes in the immune cell that weaken the function of the immune cell by at least 30, 40, 50, 60, 70, 80, 90, or 95% as compared to the immune cell in the absence of the genetic disruption agent. In some embodiments, the genetic disruption agent reduces the expression of a gene that increases reactions of the immune cell with a molecule which suppresses immune response of the immune cell. In some embodiments, the genetic disruption agent reduces the expression of a gene that encodes an immune checkpoint receptor or ligand. In some embodiments, the genetic disruption agent reduces the expression of a gene PD1, TGFBR1, or TGFBR2.

In some embodiments, the genetic disruption agent reduces the expression of the genes that weaken the function of the immune cell by RNA interference (RNAi). In some embodiments, more than one genetic disruption agents reduce the expression of at least two genes that weaken the function of the immune cell in the immune cell by RNAi. In some embodiments, the genetic disruption agents target different genes which weaken the function of the immune cell, or in any combination thereof. In some embodiments, the RNAi is mediated by a short hairpin RNA (shRNA). In some embodiments, the RNAi is mediated by more than one shRNAs.

In some embodiments, the RNAi is mediated by two shRNAs. In some embodiments, two shRNAs target PD-1 and TGFBR1, respectively. In some embodiments, two shRNAs target PD-1 and TGFBR2, respectively.

In some embodiments, base sequences encoding the shRNAs comprise sequences selected from the groups consisting of SEQ ID NOs: 1-12 and 13-23. In some embodiments, base sequences encoding the shRNAs comprise sequences selected from the groups consisting of SEQ ID NOs: 43-47 and 13-23.

In some embodiments, the expression of different shRNA is respectively regulated by different promoters. In some embodiments, the expression of two different shRNA is respectively regulated by two different promoters. In some embodiments, the two different promoters are RNA polymerase III promoters. In some embodiments, the two promoters are U6 promoters derived from different species. In some embodiments, the two promoters are oriented in different directions from each other.

In some embodiments, the genetically engineered antigen receptor and the genetic disruption agent are each expressed from a vector. In some embodiments, the genetically engineered antigen receptor and the genetic disruption agent are expressed from the same vector. In some embodiments, the vector is selected from the group consisting of DNA, RNA, plasmid, lentivirus vector, adenovirus vector, and retrovirus vector. In some embodiments, the vector is lentivirus vector.

The immune cell may be characterized in that it is selected from lymphocytes, such as killer T cells, helper T cells, gamma delta T cells and B cells, natural killer cells, mast cells, eosinophils, basophils; and the phagocytic cells include macrophages, neutrophils, and dendritic cells. The T cells include CD4+ T cells and CD8+ T cells. In some embodiments, the B-cell lymphoma is diffuse large B cell lymphoma (DLBCL), primary mediastinal B-cell lymphoma (PMBL), Hodgkin lymphoma (HL), non-Hodgkin lymphoma, mediastinal gray zone lymphoma, or nodular sclerosis HL. In some embodiments, the T-cell lymphoma is anaplastic large cell lymphoma (ALCL), peripheral T cell lymphoma not otherwise specified (PTCL-NOS), or angioimmunoblastic T cell lymphoma (AITL). In a preferred embodiment, the immune cell may be selected from human-derived T cells or T lymphocytes and natural killer (NK) cells. In some embodiments, the immune cell is a T cell. In some embodiments, the T cell is a CD4+ T cell or a CD8+ T cell.

In some embodiments, the immune cells are produced from cells originally derived from a subject. In some embodiments, the subject can be a human being. In some embodiments, the human being can be a healthy donor. In other embodiments, the subject may be characterized as having a tumor or cancer, wherein an increase or variation in levels of cancer antigen targeted by the CAR or the TCR, for example, mTCR expressed in the cell is detected. In some embodiments, cells may be produced and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In some embodiments, CAR-T cells are produced. In further embodiments, the production of CAR-T cells comprises a step of providing peripheral blood monoclonal cells. In further embodiments, the peripheral blood monoclonal cells can be separated from whole blood samples. In some embodiment, the production of CAR-T cells described herein comprises a step of stimulation of the peripheral blood monoclonal cells using antibodies. By way of example, the agent providing the primary stimulation signal is an anti-CD3 antibody or an antigen-binding fragment thereof and the agent providing the costimulatory signal is an anti-CD28 antibody or antigen-binding fragment thereof. In some embodiments, the production of CAR-T cells described herein comprises a step of transduction of CAR, wherein the CAR can target any target described herein and any other known targets of CAR, for example the CAR can be a CD19 CAR that targets CD19. In further embodiments, CAR-T cells produced are isolated.

Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-y, IL-4, IL-7, GM-CSF, IL-I 0, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). Several cycles of stimulation may also be desired such that culture time of T cells can be 60 days or more.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree. Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Various assays can be used to evaluate the CAR-T cells, such as but not limited to, the ability to expand following antigen stimulation, sustain T cell expansion in the absence of re-stimulation, and anti-cancer activities in appropriate in vitro and animal models. Assays are described in further detail below.

In some embodiments, western blot analysis of CAR expression in primary T cells can be used to detect the presence of monomers and dimers. See, e.g., Milone et al., Molecular Therapy 17(8): 1453-1464 (2009).

In some embodiments, in vitro expansion of CAR+T cells following antigen stimulation can be measured by flow cytometry. For example, a mixture of CD4+ and CD8+ T cells are stimulated with αCD³/αCD28 aAPCs followed by transduction with lentiviral vectors expressing GFP under the control of the promoters to be analyzed. Exemplary promoters include the CMV IE gene, EF-1 a, ubiquitin C, or phosphoglycerokinase (PGK) promoters. GFP fluorescence is evaluated on day 6 of culture in the CD4+ and/or CD8+ T cell subsets by flow cytometry. See, e.g., Milone et al., Molecular Therapy 17(8): 1453-1464 (2009). Alternatively, a mixture of CD4+ and CD8+ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated, e.g., with K562 cells expressing hCD32 and 4-1BBL in the presence of anti-CD3 and anti-CD28 antibody (K562-BBL-3/28) following washing. Exogenous IL-2 is added to the cultures every other day at 100 IU/ml. GFP+ T cells are enumerated by flow cytometry using bead-based counting. See, e.g., Milone et al., Molecular Therapy 17(8): 1453-1464 (2009). Sustained CAR+ T cell expansion in the absence of re-stimulation can also be measured.

Assessment of cell proliferation and cytokine production has been previously described, e.g., at Milone et al., Molecular Therapy 17(8): 1453-1464 (2009). Briefly, assessment of CAR-mediated proliferation are performed in microtiter plates by mixing washed T cells with target cells, such as K562-Meso, Ovcar3, Ovcar8, SW1990, Panc02.03 cells or CD32 and CD137 (KT32-BBL) for a final T-cell:target cell ratio of 1:1. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8+ T cell expansion ex vivo. T cells are enumerated in cultures using CountBright™ fluorescent beads (Invitrogen, Carlsbad, Calif.) and flow cytometry as described by the manufacturer. CAR+ T cells are identified by GFP expression using T cells that are engineered with eGFP-2A linked CAR-expressing lentiviral vectors. CD4+ and CD8+ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences, San Diego, Calif.) according the manufacturer's instructions. Fluorescence is assessed using a FACScalibur flow cytometer, and data is analyzed according to the manufacturer's instructions.

Cytotoxicity can be assessed by methods described herein, e.g., in the examples, or by a standard ⁵¹Cr-release assay (Milone et al., Molecular Therapy 17(8): 1453-1464 (2009)). Briefly, target cells (e.g., BHK or CHO cells) are loaded with 51 Cr (as NaCr04, New England Nuclear, Boston, Mass.) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition. Alternative cytotoxicity assays may also be used, such as flow based cytotoxicity assays.

Other assays, including those described in the Example section herein as well as those that are known in the art can also be used to evaluate the CAR-T cells produced herein.

In some embodiments, the immune cells produced herein can be involved in immune response to diseases wherein the antigens targeted by the two types of shRNA and the CAR or the TCR, for example, mTCR are expressed. In further embodiments, the immune cells can be used to provide a pharmaceutical composition for immune therapy of human patients. In some embodiments, the pharmaceutical composition can show therapeutic effect on the target illness without the need for immune checkpoint inhibitors, which may cause severe adverse reactions and burden the patient additionally with high costs.

In some embodiments, the immune cells can be used as immune cell therapeutic agents; such immune cells are normally used for the treatment of cancers but are not limited thereto. In further embodiments, to make these immune cells recognize cancers, they are modified to express cell surface receptors which target cancer antigens.

In another aspect, provided herein are compositions comprising the engineered immune cells described above.

5. Treatment Using CAR-T Cells that Targeting One or More Immune Checkpoints

As provided herein, a Two-in-One vector includes a base sequence encoding two types of short hairpin RNA (shRNA) which inhibit the expression of at least two genes that weaken the function of immune cells, and a base sequence encoding any one of a chimeric antigen receptor (CAR) and a T cell receptor (TCR) for example, a monoclonal T cell receptor (mTCR). Using the vector according to one embodiment, immune cells can be produced, wherein the immune cells have reduced immune checkpoint receptor expression and which express CAR or TCR, for example, mTCR specific to target molecules. Said immune cells can be used to provide a pharmaceutical composition for immune therapy.

A variety of diseases may be ameliorated by introducing immune cells as described herein to a subject suitable for adoptive immune therapy. In some embodiments, the produced CAR-T cells as provided is for allogeneic adoptive cell therapies. Additionally provided herein are therapeutic use of the compositions described herein, comprising introducing the composition to a subject suitable for adoptive cell therapy, wherein the subject has an autoimmune disorder; a hematological malignancy; a solid tumor; or an infection associated with HIV, RSV, EBV, CMV, adenovirus, or BK polyomavirus.

Examples of hematological malignancies include, but are not limited to, acute and chronic leukemias (acute myelogenous leukemia (AML), acute lymphoblastic leukemia (ALL), chronic myelogenous leukemia (CML), lymphomas, non-Hodgkin lymphoma (NHL), Hodgkin's disease, multiple myeloma, and myelodysplastic syndromes. Examples of solid cancers include, but are not limited to, cancer of the brain, prostate, breast, lung, colon, uterus, skin, liver, bone, pancreas, ovary, testes, bladder, kidney, head, neck, stomach, cervix, rectum, larynx, and esophagus. Examples of various autoimmune disorders include, but are not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barré syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, systemic lupus, erythematosus, some forms of thyroiditis, some forms of uveitis, vitiligo, granulomatosis with polyangiitis (Wegener's). Examples of viral infections include, but are not limited to, HIV—(human immunodeficiency virus), HSV—(herpes simplex virus), KSHV—(Kaposi's sarcoma-associated herpesvirus), RSV—(Respiratory Syncytial Virus), EBV—(Epstein-Ban virus), CMV—(cytomegalovirus), VZV (Varicella zoster virus), adenovirus-, a lentivirus-, a BK polyomavirus-associated disorders.

Acute leukemia is characterized by the rapid proliferation of immature blood cells. This crowding makes the bone marrow unable to produce healthy blood cells. Acute forms of leukemia can occur in children and young adults. In fact, it is a more common cause of death for children in the U.S. than any other type of malignant disease Immediate treatment is required in acute leukemia due to the rapid progression and accumulation of the malignant cells, which then spill over into the bloodstream and spread to other organs of the body. Central nervous system (CNS) involvement is uncommon, although the disease can occasionally cause cranial nerve palsies. Chronic leukemia is distinguished by the excessive buildup of relatively mature, but still abnormal, blood cells. Typically taking months to years to progress, the cells are produced at a much higher rate than normal cells, resulting in many abnormal white blood cells in the blood. Chronic leukemia mostly occurs in older people, but can theoretically occur in any age group. Whereas acute leukemia must be treated immediately, chronic forms are sometimes monitored for some time before treatment to ensure maximum effectiveness of therapy. Furthermore, the diseases are classified into lymphocytic or lymphoblastic, which indicate that the cancerous change took place in a type of marrow cell that normally goes on to form lymphocytes, and myelogenous or myeloid, which indicate that the cancerous change took place in a type of marrow cell that normally goes on to form red cells, some types of white cells, and platelets (see lymphoid cells vs. myeloid cells).

Acute lymphocytic leukemia (also known as acute lymphoblastic leukemia, or ALL) is the most common type of leukemia in young children. This disease also affects adults, especially those aged 65 and older. Chronic lymphocytic leukemia (CLL) most often affects adults over the age of 55. It sometimes occurs in younger adults, but it almost never affects children. Acute myelogenous leukemia (also known as acute myeloid leukemia, or AML) occurs more commonly in adults than in children. This type of leukemia was previously called “acute nonlymphocytic leukemia.” Chronic myelogenous leukemia (CML) occurs mainly in adults. A very small number of children also develop this disease.

Lymphoma is a type of cancer that originates in lymphocytes (a type of white blood cell in the vertebrate immune system). There are many types of lymphoma. According to the U.S. National Institutes of Health, lymphomas account for about five percent of all cases of cancer in the United States, and Hodgkin's lymphoma in particular accounts for less than one percent of all cases of cancer in the United States. Because the lymphatic system is part of the body's immune system, patients with a weakened immune system, such as from HIV infection or from certain drugs or medication, also have a higher incidence of lymphoma.

In the 19th and 20th centuries the affliction was called Hodgkin's Disease, as it was discovered by Thomas Hodgkin in 1832. Colloquially, lymphoma is broadly categorized as Hodgkin's lymphoma and non-Hodgkin lymphoma (all other types of lymphoma). Scientific classification of the types of lymphoma is more detailed. Although older classifications referred to histiocytic lymphomas, these are recognized in newer classifications as of B, T, or NK cell lineage.

In some embodiments, the cancer treated in accordance with the methods provided herein expresses EGFR. In some embodiments, the cancer treated in accordance with the methods described herein is lung cancer. In specific embodiments, the cancer is small cell lung cancer or non-small cell lung cancer. In other specific embodiments, the cancer is skin cancer. In some embodiments, the cancer treated in accordance with the methods described herein is non-Hodgkin's lymphoma.

When the pharmaceutical composition are administered to a patient having a cancer wherein the target molecule of the CAR or TCR, for example, mTCR, is expressed, the pharmaceutical composition can recognize the cancer and have immune activity without the activation of genes which weaken the function of immune cells with regard to cancer cells, and without problems such as exhaustion due to activation-inhibiting signaling caused thereby.

In some embodiment, the above pharmaceutical composition comprising immune cells is able to more effectively suppress the expression of immune checkpoint receptors while simultaneously maximizing the effectiveness of anti-cancer immune cell therapy wherein chimeric antigen receptors can function. In further embodiments, as cells wherein expression of immune checkpoint receptors is suppressed as described in the above are used in the pharmaceutical composition, it is possible to eliminate the severe and systemic adverse reactions such as cytokine release syndrome or autoimmune symptoms which may result from using a separate inhibitor for immune checkpoint receptor, as well the burden due to the increased cost of treatment resulting from the concurrent use of expensive antibody therapies with cell therapy.

As it is self-evident that, in addition to the cells, other pharmaceutically acceptable salts, carriers, excipients, vehicles and other additives, etc. which may further improve immune response may be added to the pharmaceutical composition, a detailed explanation thereof shall be omitted.

In some embodiments, targeting certain two immune checkpoints can produce surprisingly high anti-tumor effect.

In one aspect, a composition described herein can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of a composition described herein, alone or in combination with other active agents, calculated in an amount sufficient to produce the desired effect, in association with a pharmaceutically acceptable diluent, carrier, or vehicle, where appropriate. The specifications for the novel unit dosage forms of cells or compositions described herein depend on the particular pharmacodynamics associated with the pharmaceutical composition in the particular subject.

In some embodiments, the preferred pharmaceutical dosage form for the cells or compositions described herein may be determined based on the content of the present disclosure and general knowledge of formulation techniques and according to the intended administration pathway, method of delivery and the target dose. The method of administration notwithstanding, the effective dose may be calculated in according to the patient's body weight, surface area or organ size. Calculations to determine the appropriate administration doses for therapy using the respective dosage forms stated in the present specification, as well as additional purification, are carried out on a daily basis in the art, and are included within the scope of work carried out on a daily basis in the art. The appropriate administration doses may be identified through use of appropriate dose-response data.

Pharmaceutical compositions described herein can be used alone or in combination with other known agents useful for treating cancer. Whether delivered alone or in combination with other agents, pharmaceutical compositions described herein can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration. Exemplary route of administration to a subject includes intravenous (IV) injection, and regional (intratumoral, intraperitoneal) administration. In some embodiment, the pharmaceutical composition can be administered via infusion into a solid tumor.

In some embodiments, in addition to the genomically engineered immune cells as provided herein, additional therapeutic agent comprising an antibody, or an antibody fragment that targets an antigen associated with a condition, a disease, or an indication may be used with these effector cells in a combinational therapy. In some embodiments, the antibody is a monoclonal antibody. In some embodiments, the antibody is a humanized antibody, a humanized monoclonal antibody, or a chimeric antibody. In some embodiments, the antibody, or antibody fragment, specifically binds to a viral antigen. In other embodiments, the antibody, or antibody fragment, specifically binds to a tumor antigen. In some embodiments, the antibodies suitable for combinational treatment as an additional therapeutic agent to the administered genomically engineered immune cells include, but are not limited to, anti-CD20 (rituximab, veltuzumab, ofatumumab, ublituximab, ocaratuzumab, obinutuzumab), anti-HER2 (trastuzumab, pertuzumab), anti-CD52 (alemtuzumab), anti-EGFR (certuximab), anti-GD2 (dinutuximab), anti-PDL1 (avelumab), anti-CD38 (daratumumab, isatuximab, MOR202), anti-CD123 (7G3, CSL362), anti-SLAMF7 (elotuzumab); and their humanized or Fc modified variants or fragments, or their functional equivalents and biosimilars.

Desirably an effective amount or sufficient number of the isolated transduced T cells is present in the composition and introduced into the subject such that long-term, specific, anti-tumor responses are established to reduce the size of a tumor or eliminate tumor growth or regrowth than would otherwise result in the absence of such treatment. Desirably, the amount of transduced T cells reintroduced into the subject causes a 10%, 20%, 30%, 40%, 50%, 60%, 70%, 80%, 90%, 95%, 98%, or 100% decrease in tumor size when compared to otherwise same conditions wherein the transduced T cells are not present.

Accordingly, the amount of transduced T cells administered should take into account the route of administration and should be such that a sufficient number of the transduced T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated at least from about 1×10⁶ to about 1×10⁹ transduced T cells, even more desirably, from about 1×10⁷ to about 5×10⁸ transduced T cells, although any suitable amount can be utilized either above, e.g., greater than 5×10⁸ cells, or below, e.g., less than 1×10⁷ cells. The dosing schedule can be based on well-established cell-based therapies (see, e.g., Topalian and Rosenberg, 1987; U.S. Pat. No. 4,690,915), or an alternate continuous infusion strategy can be employed.

These values provide general guidance of the range of transduced T cells to be utilized by the practitioner upon optimizing the methods described herein. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

Any of the compositions described herein may be comprised in a kit. In some embodiments, the CAR T-cells are provided in the kit, which also may include reagents suitable for expanding the cells, such as media, aAPCs, growth factors, antibodies (e.g., for sorting or characterizing CAR T-cells) and/or plasmids encoding CARs or transposase.

In a non-limiting example, a chimeric receptor expression construct, one or more reagents to generate a chimeric receptor expression construct, cells for transfection of the expression construct, and/or one or more instruments to obtain allogeneic cells for transfection of the expression construct (such an instrument may be a syringe, pipette, forceps, and/or any such medically approved apparatus).

In some embodiments, an expression construct for eliminating endogenous TCR α/β expression, one or more reagents to generate the construct, and/or CAR⁺ T cells are provided in the kit. In some embodiments, there includes expression constructs that encode zinc finger nuclease(s). In some aspects, the kit comprises reagents or apparatuses for electroporation of cells.

The kits may comprise one or more suitably aliquoted compositions described herein or reagents for generating compositions as described herein. The components of the kits may be packaged either in aqueous media or in lyophilized form. The container means of the kits may include at least one vial, test tube, flask, bottle, syringe, or other container means, into which a component may be placed, and in certain embodiments, suitably aliquoted. Where there is more than one component in the kit, the kit also will generally contain a second, third, or other additional container into which the additional components may be separately placed. However, various combinations of components may be comprised in a vial. The kits described herein also will typically include a means for containing the chimeric receptor construct and any other reagent containers in close confinement for commercial sale. Such containers may include injection or blow molded plastic containers into which the desired vials are retained, for example.

In one aspect, provided herein are pharmaceutical compositions comprising the immune cell described above and a pharmaceutically acceptable carrier. In another aspect, provided herein are pharmaceutical compositions for immune therapy of human patients comprising the immune cells described above. In some embodiments, the immune cell is originally derived from the patient. In some embodiments, the patient has a tumor or cancer in which an increase or variation in levels of cancer antigen targeted by the CAR or TCR, for example, mTCR, expressed in the cell is detected.

In another aspect, provided herein are methods. In some embodiment, provided are methods of treatment comprising administering to a subject having a disease or a condition the immune cell described above or the composition described above. In some embodiments, the genetically engineered antigen receptor specifically binds to an antigen associated with the disease or the condition. In some embodiments, the disease or the condition is a cancer or a tumor.

In another aspect, provided herein are immune cells and compositions. In some embodiments, provided are immune cells and compositions described above for use in treating a disease or a condition.

In another aspect, provided herein is use of the immune cells or compositions. In some embodiments, provided is use of the immune cells or compositions described above in the manufacture of a medicament for use in a method for treating a disease or a condition. In some embodiments, the genetically engineered antigen receptor specifically binds to an antigen associated with the disease or the condition. In some embodiments, the disease or the condition is a cancer or a tumor.

The above description of the present invention is intended to be exemplary, and persons with ordinary skill in the art shall understand that the present invention may be easily modified into certain other forms without changing the technical idea or essential characteristics of the present invention. Accordingly, the embodiments described in the above shall be understood ad being exemplary and not limiting in all aspects. For example, respective component elements which are described as being integrated may be carried out separately, and likewise, component elements which are described as being carried out separately may be carried out in an integrated manner.

The scope of the present invention is represented by the appended claims, and all modified or changed forms derived from the meaning and scope of the claims and concepts equivalent thereto shall be interpreted as being included within the scope of the present invention.

TABLE 1
Table of Sequences

| SEQ ID | | |
|---|---|---|
| NO | Name | Sequence |
| 1 | shRNA against | GGAGAAAGAATGACGAGAACA |
| 2 | TGFBR2 | GCTCCAGAAGTCCTAGAATCC |
| 3 | | GGAGGAGAAGATTCCTGAAGA |
| 4 | | GCATAGAGGCGCCTAGAAATT |
| 5 | | GGTTCCTGTGTGCCCTTATTT |
| 6 | | GCGCCTAGAAATTCCACTTGC |
| 7 | | TCCTGCATGAGCAACTGCA |
| 8 | | GCTTCTCCAAAGTGCATTA |
| 9 | | CCACGTGTGCCAACAACAT |
| 10 | | GCTCCAATATCCTCGTGAA |
| 11 | | CCTGTGTCGAAAGCATGAA |
| 12 | | GCTCCCTAAACACTACCAA |
| 13 | shRNA against | tggaacccattcctgaaatta |
| 14 | PD1 | ggaacccattcctgaaattat |
| 15 | | gaacccattcctgaaattatt |
| 16 | | acccattcctgaaattattta |
| 17 | | cccattcctgaaattatttaa |
| 18 | | ccttccctgtggttctattat |
| 19 | | cttccctgtggttctattata |
| 20 | | ttccctgtggttctattatat |
| 21 | | tccctgtggttctattatatt |
| 22 | | ccctgtggttctattatatta |
| 23 | | cctgtggttctattatattat |
| 24 | TGFBR2 | acgtgttgagagatcgagg |
| | primer | |
| | Forward | |
| 25 | TGFBR2 | cccagcactcagtcaacgtc |
| | primer Reverse | |
| 26 | PD-1 primer | cctccacctttacacatgcc |
| | Forward | |
| 27 | PD-1 primer | cttactgcctcagcttccct |
| | Reverse | |
| 28 | Cloning | ggtatactctagacatatggctagcactagtcaaaaacctgtggttc |
| 29 | primers | ttgtaccgttaacgatccgacgccgc |
| 30 | | tgactagtcaaaaacctgtggttctattatattattctcttgaaataatataatagaaccacag |
| | | gcggtgtttcgtcctttccacaagatatataaagccaa |
| 31 | | gtaccgttaacaaggtcgggcaggaagagggcctatttcccatgattcct |
| 32 | | aggactagtcaaaaaggaattcgctcagaagaaatctct |
| 33 | | ctagctagcgatccgacgccgccatct |
| 34 | | atgttaaccaaaaacctgtggttctattatattattctcttg |
| 35 | | tcactagtaaggtcgggcaggaagagggcctatt |
| 36 | | taggccctcactagtgatccgacgccgcc |
| 37 | | ctagctagccaaaaaggaattcgctcagaagaaatctc |
| 38 | shPD-1-mU6- | gttaacgatccgacgccgccatctctaggcccgcgccggccccctcgcacagacttgtg |
| | MCS base | ggagaagctcggctactcccctgccccggttaatttgcatataatatttcctagtaactatag |
| | sequence | aggcttaatgtgcgataaaagacagataatctgttctttttaatactagctacattttacatgat |
| | | aggcttggatttctataagagatacaaatactaaattattattttaaaaaacagcacaaaagg |
| | | aaactcaccctaactgtaaagtaattgtgtgttttgagactataaatatcccttggagaaaag |
| | | ccttgtttgcctgtggttctattatattatttcaagagaataatataatagaaccacaggtttttg |
| | | actagtgctagccatatgtctagagtatac |
| 39 | hU6-shPD-1 | gttaacaaggtcgggcaggaagagggcctatttcccatgattccttcatatttgcatatacg |
| | base sequence | atacaaggctgttagagagataattagaattaatttgactgtaaacacaaagatattagtac |
| | | aaaatacgtgacgtagaaagtaataatttcttgggtagtttgcagttttaaaattatgttttaaa |
| | | atggactatcatatgcttaccgtaacttgaaagtatttcgatttcttggctttatatatcttgtgg |
| | | aaaggacgaaacaccgcctgtggttctattatattatttcaagagaataatataatagaacc |
| | | acaggtttttg |
| 40 | CD22-CAR | QVQLQQSGPGLVKPSQTLSLTCAISGDSVSSNSAAWNWI |
| | | RQSPSRGLEWLGRTYYRSKWYNDYAVSVKSRITINPDT |
| | | SKNQFSLQLNSVTPEDTAVYYCAREVTGDLEDAFDIWG |
| | | QGTMVTVSSGGGGSGGGGSGGGGSDIQMTQSPSSLSAS |
| | | VGDRVTITCRASQTIWSYLNWYQQRPGKAPNLLIYAAS |
| | | SLQSGVPSRFSGRGSGTDFTLTISSLQAEDFATYYCQQSY |
| | | SIPQTFGQGTKLEITTTPAPRPPTPAPTIASQPLSLRPEACR |
| | | PAAGGAVHTRGLDFACDIYIWAPLAGTCGVLLLSLVITL |
| | | YCKRGRKKLLYIFKQPFMRPVQTTQEEDGCSCRFPEEEE |
| | | GGCELRVKFSRSADAPAYKQGQNQLYNELNLGRREEY |
| | | DVLDKRRGRDPEMGGKPRRKNPQEGLYNELQKDKMA |
| | | EAYSEIGMKGERRRGKGHDGLYQGLSTATKDTYDALH |
| | | MQALPPR |
| 41 | CD19-CAR | MALPVTALLLPLALLLHAARPDIQMTQTTSSLSASLGDR |
| | | VTISCRASQDISKYLNWYQQKPDGTVKLLIYHTSRLHSG |
| | | VPSRFSGSGSGTDYSLTISNLEQEDIATYFCQQGNTLPYT |
| | | FGGGTKLEITGGGGSGGGGSGGGGSEVKLQESGPGLVA |
| | | PSQSLSVTCTVSGVSLPDYGVSWIRQPPRKGLEWLGVIW |
| | | GSETTYYNSALKSRLTIIKDNSKSQVFLKMNSLQTDDTA |
| | | IYYCAKHYYYGGSYAMDYWGQGTSVTVSSTTTPAPRPP |
| | | TPAPTIASQPLSLRPEACRPAAGGAVHTRGLDFACDIYI |
| | | WAPLAGTCGVLLLSLVITLYCKRGRKKLLYIFKQPFMRP |
| | | VQTTQEEDGCSCRFPEEEEGGCELRVKFSRSADAPAYK |
| | | QGQNQLYNELNLGRREEYDVLDKRRGRDPEMGGKPRR |
| | | KNPQEGLYNELQKDKMAEAYSEIGMKGERRRGKGHDG |
| | | LYQGLSTATKDTYDALHMQALPPR |
| 42 | Delta | MGAGATGRAMDGPRLLLLLLLGVSLGGAKEACPTGLY |
| | LNGFR_P2A_ | THSGECCKACNLGEGVAQPCGANQTVCEPCLDSVTFSD |
| | CD19-CAR | VVSATEPCKPCTECVGLQSMSAPCVEADDAVCRCAYG |
| | | YYQDETTGRCEACRVCEAGSGLVFSCQDKQNTVCEECP |
| | | DGTYSDEANHVDPCLPCTVCEDTERQLRECTRWADAEC |
| | | EEIPGRWITRSTPPEGSDSTAPSTQEPEAPPEQDLIASTVA |
| | | GVVTTVMGSSQPVVTRGTTDNLIPVYCSILAAVVVGLV |
| | | AYIAFKRWGSGATNFSLLKQAGDVEENPGPALPVTALL |
| | | LPLALLLHAARPDIQMTQTTSSLSASLGDRVTISCRASQ |
| | | DISKYLNWYQQKPDGTVKLLIYHTSRLHSGVPSRFSGSG |
| | | SGTDYSLTISNLEQEDIATYFCQQGNTLPYTFGGGTKLEI |
| | | TGGGGSGGGGSGGGGSEVKLQESGPGLVAPSQSLSVTC |
| | | TVSGVSLPDYGVSWIRQPPRKGLEWLGVIWGSETTYYN |
| | | SALKSRLTIIKDNSKSQVFLKMNSLQTDDTAIYYCAKHY |
| | | YYGGSYAMDYWGQGTSVTVSSTTTPAPRPPTPAPTIAS |
| | | QPLSLRPEACRPAAGGAVHTRGLDFACDIYIWAPLAGT |
| | | CGVLLLSLVITLYCKRGRKKLLYIFKQPFMRPVQTTQEE |
| | | DGCSCRFPEEEEGGCELRVKFSRSADAPAYKQGQNQLY |
| | | NELNLGRREEYDVLDKRRGRDPEMGGKPRRKNPQEGL |
| | | YNELQKDKMAEAYSEIGMKGERRRGKGHDGLYQGLST |
| | | ATKDTYDALHMQALPPR |
| 43 | shRNA against | ggagattgttggtacccaagg |
| 44 | TGFBR1 | gcagctaggcttacagcattg |
| 45 | | ggtcctttctgtgcactatga |
| 46 | | ggtggtagctaaagaacattc |
| 47 | | ggacctgtctacaggtgatct |
| 48 | TGFBR1 | acatgattcagccacagatacc |
| | primer | |
| | Forward | |
| 49 | TGFBRI | gcatagatgtcagcacgtttg |
| | primer reverse | |

### EXAMPLES

Examples related to the present invention are described below. In most cases, alternative techniques can be used. The examples are intended to be illustrative and are not limiting or restrictive to the scope of the invention.

### Example 1: General Methods

Cell lines and culture. Nalm-6, Nalm-6GL (expressing GFP and firefly luciferase), K562, K562-CD19, IM-9, Raji, Daudi cell lines are cultured in RPMI-1640 supplemented with 10% heat-inactivated fetal bovine serum and 2 mM L-glutamine and 1% penicillin/streptomycin in a humidified incubator with 5% CO₂ at 37 ° C. Lenti-X™ 293T Cell Line (Takara) is maintained in DMEM supplemented with 10% heat-inactivated fetal bovine serum (FBS), 2 mM L-glutamine, 0.1 mM nonessential amino acids, 1 mM sodium pyruvate, and 1% penicillin/streptomycin. To generate the CD19⁺PD-L1⁺ cell lines, K562-CD19 or NALM-6-GL cells are transduced with lentivirus encoding human PD-L1 (NM_014143.3). The Nalm-6-PDL1-CD80 cell line was generated by transduction of Nalm-6-PDL1 cells with lentivirus encoding human CD80 (NM 005191.3).

Plasmid construction. Construction of the pLV-CD19-BBz vector containing the anti-CD19 scFv (FMC63), CD8α hinge and transmembrane region, and the cytoplasmic domains of 4-1BB(CD137) and CD3ζ, has been previously described (PNAS, 2016, Ma JSY). To generate pLV-CD19-28z, the cytoplasmic domain of 4-1BB is replaced with the sequence of human CD28 costimulatory domain. For the detection and purification of transduced CAR-T cells, ΔLNGFR (cytoplasmic domain truncated CD271) sequence is amplified from pMACS-ΔLNGFR (Milteny Biotec) vector, and inserted in front of the CAR-Transgene via P2A sequence, to yield pLV-ΔLNGFR-CD19-28z or pLV-ΔLNGFR-CD19-BBz.

To generate Two-in-One LV vectors that encode both CAR and shRNA expression cassettes, shRNA expressing cassette containing shRNA (link sequence; TTCAAGAGA, termination sequence; TTTTT) and PolIII promoters (mU6, hU6 or hH1) are synthesized and subcloned into CAR-encoding LV vectors upstream of central polypurine tract (cPPT). For generation of Dual Two-in-One vectors expressing two shRNAs by different promoters (mU6 and hU6), BstZ171-Xba1-Nde1-Bmt1-Spe1 MCS sequence is inserted into pLV-hU6-shPD-1_ΔLNGFR-CD19-BBz vector downstream of hU6 promoter. The second mU6-shRNA cassette fragments are subcloned into the MCS.

For establishment of reporter vectors, NFAT RE x3 sequence derived from pGL2_NFAT-Luc reporter (addgene #10959) are amplified by PCR. NF-kB-RE 5x (5′-GGGAATTTCC-3′) and miniP sequence are synthesized (IDT Technologies). EF-1a promoter of pLV-eGFP vector are replaced with these reporter fragments, to yield pLV-NFAT-RE 3x-eGFP or pLV-NF-kB-RE 5x-eGFP reporter vectors.

Selection the siRNA or shRNA sequences. The candidate sequences of 21-mer siRNAs that are specific for PD-1, TGFBR1, or TGFBR2 are designed by using BLOCK-iT™ RNAi Designer or Sfold programs before synthesizing. To analyze the expression kinetics of immune checkpoints, PBMCs are stimulated with Dynabeads Human T-Activator CD3/CD28 (Thermofisher) or 4 μg/ml anti-CD3 antibody and 2 μg/ml anti-CD28 antibody in the presence of human recombinant IL-2. The expression levels of immune checkpoints are analyzed for 12 days (on day 3, day 6, and day 12). For the selection of optimal siRNA sequences, 2 days after stimulation, PBMCs are electroporated with siRNA oligomers using Neon® Transfection System (Thermofisher). The knock-down efficiencies of siRNAs are measured two days after transfection using flow cytometry. 2-3 siRNA sequences are selected for each immune checkpoint based on their efficiency, and converted into shRNA format to generate the dual Two-in-One lentiviral vectors. To validate shRNA-mediated knock-down efficacies, the lentivirus-transduced T cells are stimulated with γ-irradiated K562-CD19 cells at a 1:1 ratio for 3 days, and then analyzed for their expression levels of immune checkpoints by flow cytometry.

Flow cytometry. The expression level of anti-CD19 CAR is analyzed by AF647-conjugated anti-mouse F(ab′)₂ antibody (115-606-072, Jackson ImmunoResearch) or biotin-conjugated rhCD19-Fc (CD9-H5259, ACRO Biosystems) coupled with AF647-conjugated streptavidin (405237, Biolegend). ΔLNGFR expression is analyzed by APC- or FITC-conjugated anti-CD271 antibody (ME20.4-1.H4; Mitenyi Biotec). Expression of immune checkpoints in CAR-T cells is measured by conventional flow cytometry using following antibodies: PD-1 (PE, clone J105; Thermofisher), TIM-3 (PE, clone 344823; R & D systems), LAG-3 (PE, clone 7H2C65; Biolgend), TIGIT (PE, clone MBSA43; Thermofisher).

PD-1, TGFBR1, and TGFBR2 expression on CAR-T cells is analyzed by intracellular flow cytometry following incubation with irradiated NALM-6 or K562-CD19 cells at an E:T ratio of 1:1 for 3 days. The cells is fixed/permeabilized with Cytofix/Cytoperm™ solution (BD Bioscience), followed by staining with anti-human CD152 (CTLA-4) antibody (PE, clone BNI3; Biolegend).

The expression of stimulatory or inhibitory immune checkpoint ligands on tumor cells is analyzed using the following antibodies: CD80 (PE, clone 2D10; Biolgend), CD86 (BV421, clone 2331(FUN-1); BD Bioscience), PD-L1 (APC, clone 29E.2A3; Biolgend), HLA-DR (PE, clone L243; Biolgend), CD112 (PE, clone TX31; Biolgend), CD155 (PE, clone SKII.4; Biolgend), TGFBR1 (clone TB21; ThermoFisher Scientific) and TGFBR2 (R&D Biosystems).

For the analysis of the effect of TGF-β on the expression of PD-1, CAR-T cells is stimulated with irradiated NALM-6 cells for 3 days in the presence of 10 ng/ml recombinant human TGF-β1 (R & D systems) before measuring PD-1 expression by flow cytometry.

Analysis of the phosphorylation status of SMAD2/3 by intracellular flow cytometry. To determine phosphorylation status of SMAD2/3, CAR-T cells is incubated with NALM-6 cells at an E:T cell ratio of 1:1 for 4 hr or 24 hr. The cells is fixed with Lyse/Fix Buffer (BD Bioscience), followed by permeabilization with Perm Buffer III (BD Bioscience). The phosphorylation status on LNGFR⁺CAR-T cells is determined with anti-human Smad2(pS465/pS467)/Smad3 (pS423/pS425) antibody (PE, clone 072-670; BD Bioscience).

Detection of induced regulatory T cells. Induction of regulatory T cells (CD4⁺CD25⁺FOXP3⁺), generated from CAR-T cells, is analyzed by intracellular staining after co-culture with NALM-6 cells for 3 days. The cells that were fixed and permeabilzed with Foxp3/Transcription Factor Staining Buffer (Thermofisher) are stained with following antibodies: anti-CD4 (BV605, clone OKT4; Biolgend), anti-CD25 (FITC, clone VT-072; Biolgend), and anti-FOXP3 (APC, clone 236A/E7; Thermofisher).

CAR-T proliferation assay. CAR-T cells expressing ΔLNGFR surface marker are sorted by magnetic beads (Miltenyi Biotec). LNGFR⁺CAR-T cells (1×10⁶; >95% purity) are stimulated with γ-irradiated K562-CD19-PDL1 cells (1×10⁶) every 6 day in absence of cytokines. Fold-expansion of CAR-T cells is calculated by cell counting on day 6, 12, and 18 using trypan blue exclusion.

In vitro cytotoxicity of CAR-T cells. The cytotoxicity of CAR-T cells is determined by using Incucyte S3 live cell analysis system. NALM-6 or NALM-6-PDL1 target cells that constitutively express GFP are plated into a 96-well plate at a density of 1×10⁵ cells per well in triplicate. LNGFR⁺CAR-T cells are added into each well at an E:T ratio of 1:1, 0.3:1, 0.1:1. The real-time change of GFP intensity of each wells is recorded every 2 hour as green object integrated intensity (Avg. mean GFP intensity×μm²/wells). The percentage of relative green object integrated intensity is calculated by the following formula: (total integrated GFP intensity of at each time point/total integrated GFP intensity of at the start time point)*100.

NFAT and NF-κB reporter assay. To determine the specific activity of NFAT transcription factor in CAR-T cells, PBMCs that are stimulated with 4 μg/ml anti-CD3 and 2 μg/ml anti-CD28 antibodies in the presence of human recombinant IL-2 (300 IU/mL) for 2 days are first transduced with the lentivirus encoding the NFAT-RE x3-eGFP reporter gene. Eight days after transduction, the total cells are re-stimulated with anti-CD3 and anti-CD28 antibody in the presence of human recombinant IL-2 (300 IU/mL) for 2 days. The activated cells are split into two separated wells and transduced with different CAR-encoding lentivirus (CD19-28z or CD19-BBz). After 6 days, the total cells in each well are co-incubated with NALM-6 cells at a 1:1 ratio. The reporter activity of NFAT in CAR-T cells is determined by the gMFI value of the eGFP signal within the LNGFR⁺ CAR-T populations (20˜25% in total CD3+ cells) at 24- and 48-hour time point. The specific activity of NF-κB transcription factor in CAR-T cells is measured following a similar procedure but using the lentivirus encoding NF-κB-RE x5-eGFP reporter gene.

Quantitative real-time PCR. 3×10⁶ LNGFR⁺ G28z or GBBz CAR-T cell are co-cultured with CD19⁺ NALM-6 target cells at a 1:1 ratio. After 4 and 48 hours of co-culture, LNGFR⁺CAR-T cells are sorted using a MoFlo Astrios sorter (Beckman Coulter). The mRNA from LNGFR⁺ CAR-T cells is extracted by RNeasy mini kit (Qiagen) and reverse transcribed into cDNA using QuantiTect Reverse Transcription Kit (Qiagen). Quantitative Real-Time PCR is performed with the SYBR protocol using CFX96 Real-Time PCR Detection System (Biorad) and SYBR Green Realtime PCR Master Mix (TOYOBO). The primer sequences for detection of TGFBR1 comprise SEQ ID NOs. 48 and 49. The primer sequences for detection of PD-1 comprise SEQ ID NOs.: 26 and 27. The primer sequences for detection of TGFBR2 comprise SEQ ID NOs.: 24 and 25.

The amount of target mRNA is normalized to the endogenous reference 18s rRNA: ΔCt (sample)=Ct (gene of target)−Ct (18s rRNA). The comparative Ct method is applied to analyze the relative fold change of the target mRNA compared with the unstimulated condition based on the following equation: 2^{-ΔΔCt}=2\hat{ }-(ΔCt[stimulated]−ΔCt[unstimulated]).

Animal experiments. All procedures described herein are approved by the Institutional Animal Care and Use Committee at KAIST. To establish CD 19⁺ blood cancer model, NSG mice (4 to 6 weeks of age) are intravenously injected with 1×10⁶ CD19⁺ NALM-6 leukemia cells that are engineered to express EGFP—fused firefly luciferase as well as human PDL1 (NALM6-GL-PDL1 cells). CAR-T cells are prepared from the whole blood samples of healthy donors following the procedures described above. At day 4 after transduction, CAR⁻ T cells are sorted and further expanded for 6 day prior to the injection into mice. Five days after NALM6-GL-PDL1 cell injection, 2.5 or 1×10⁶ CAR-T cells are intravenously infused to the mice. Bioluminescence imaging of NALM6-GL-PDL1 within mice is monitored with Xenogen IVIS Spectrum and the signals are quantified as radiance in the region of interest (photon/sec) using Living Image software (Perkin Elmer). For the solid tumor model, NSG mice are subcutaneously injected with 5×10⁶ IM-9 cells (CD19⁺PD-L1⁺CD155⁺). LNGFR⁺CAR-T cells intravenously infused to the mice 14 days after the injection of tumor cells (approximately 150˜300 mm³ tumor volume). Tumors are monitored every week by caliper measurement and the volume estimated by (length×width²)/2.

### Example 2: Methods of Production of TGFBR1-Targeting Dual Two-in-One Vectors Having 2 Types of shRNA Cassette Entered in the →← or the ←→ Directions

This example describes the methods of producing dual Two-in-One vectors, wherein 2 types of shRNA cassette enter in the ←→ directions (shTGFBR1-mU6←→hU6-shPD-1) or the →← directions (mU6-shTGFBR1→←shPD-1-hU6) and express shPD-1, shTGFBR1 and CD19-CAR simultaneously.

This example further describes the construction of a lentivirus, wherein the expression of the shRNA for PD-1 (hereinafter shPD-1), the shRNA for the expression of the shRNA for PD-1 and the shRNA for TGFBR1 (hereinafter shTGFBR1) and CD19-CAR are regulated by human U6 promoter (hereinafter hU6), mouse U6 promoter (hereinafter mU6), and EF1-α promoter, respectively. The plasmid wherein both types of shRNA are expressed simultaneously is prepared so that the respective shRNA cassettes are disposed in the →← direction (mU6-shTGFBR1→←shPD-1-hU6) and the ←→ direction (shTGFBR1-mU6←→hU6-shPD-1) (FIGS. 1A-B). To this end, (1) insertion of a multiple cloning site (MCS), (2) conversion of the mouse U6 promoter of shPD-1 into human U6 promoter, (3) insertion of shTGFBR1, and (4) cloning such as switching positions of shTGFBR1 and shPD-1, are performed.

To insert a MCS into the 3′ part of pLV-ΔLNGFR_P2A_CD19-CAR_mU6-shPD-1 (plasmid ID #2), primers comprising SEQ ID NOs: 28 and 29 are used to make a PCR product (416 bp) wherein the Hpa1 restriction enzyme recognition site of mU6-shPD-1 3′ is modified into a BsgZ171-Xba1-Nde-1-Bmt1-Spe1 multiple cloning site. Thereafter, the PCR product is treated with BstZ17y and Hpa1 restriction enzymes and plasmid ID #2 is treated with Hpa1 restriction enzyme and CIP, after which blunt end ligation is used to prepare a pLV-ΔLNGFR-P2A-CD19-CAR-mU6-shPD-1 MCS (plasmid ID #4) including the shPD-1-mU6-MCS base sequence (shRNA cassette SEQ ID NO: 38).

Thereafter, a plasmid is constructed so that shPD-1 is expressed under the control of the human U6 promoter instead of the mouse U6 promoter. LentiCRISPR V2 plasmid is used with primers comprising SEQ ID NOs. 30 and 31 to obtain a PCR product including the human U6 promoter. The PCR product is treated with Hpa1 and Spe1 restriction enzyme, and then ligated to plasmid ID #4 treated with Hpa1 and Spe1 restriction enzyme to prepare a pLV-ΔLNGFR_P2A_CD19-CAR_hU6-shPD-1_MSC (plasmid ID #5) comprising the hU6-shPD-1 base sequence (SEQ ID NO. 39). The mU6-shTGFBR1 cassette is inserted into plasmid ID #5 to construct a plasmid which expresses shTGFBR1 and shPD-1 simultaneously.

The PCR product including the mU6-shTGFBR1 cassette is obtained using plasmid ID #11 and primers comprising SEQ ID NOs. 32 and 33. After treating the PCR product with Bmt1 and Spe1 restriction enzymes, it is ligated to plasmid ID #5 and treated with Bmt1 and Spe1 restriction enzymes to prepare a pLV-ΔLNGFR_P2A_CD19-CAR mU6-shTGFBR1→←shPD-1-hU6 (plasmid ID #6) comprising a →← direction (mU6-shTGFBR1→←shPD-1-hU6) base sequence.

To build a plasmid wherein two types of shRNA cassettes are arranged in the ←→ directions (shTGFBR1-mU6←→hU6-shPD-1), plasmid ID #6 and primers comprising SEQ ID NOs. 34 and 35 are used to obtain a PCR product. After treating with Spe1 and Hpa1 restriction enzyme, the PCR product is inserted into plasmid ID #4 and treated with the same restriction enzymes to produce pLV-ΔLNGFR-P2A-CD19-CAR-shPD-1-hU6-MCS (plasmid ID #8). Thereafter, plasmid ID #6 and primers comprising SEQ ID NOs. 36 and 37 are used to obtain a PCR product. After treating with Bmt1 and Spe1 restriction enzyme, the PCR product is ligated to plasmid ID #8 and treated with the same restriction enzyme to ultimately prepare a pLV-ΔLNGFR-P2A-CD19-CAR_shTGFBR1-mU6←→hU6-shPD-1 (plasmid ID #9) comprising a ←→ direction (shTGFBR1-mU6←→hU6-shPD-1) base sequence.

The Two-in-One lentiviral vectors wherein two types of shRNA cassette enter in the ←→ or the →← directions produced herein can be used in the production of PD-1 KD modified CAR-T cells in the following examples.

### Example 3: Methods of Production of TGFBR2-Targeting Dual Two-in-One Vectors Having 2 Types of shRNA Cassette Enter in the →← or the ←→ Directions

This example describes the methods of producing dual Two-in-One vectors, wherein 2 types of shRNA cassette enter in the ←→ directions (shTGFBR2-mU6←→hU6-shPD-1) or the →← directions (mU6-shTGFBR2→←shPD-1-hU6) and express shPD-1, shTGFBR2 and CD19-CAR simultaneously.

This example further describes the construction of a lentivirus, wherein the expression of the shRNA for PD-1 (hereinafter shPD-1), the shRNA for TGFBR2 (hereinafter shTGFBR2) and CD19-CAR, are regulated by human U6 promoter (hereinafter hU6), mouse U6 promoter (hereinafter mU6), and EF1-α promoter, respectively. The plasmid wherein both types of shRNA are expressed simultaneously will be prepared so that the respective shRNA cassettes are disposed in the →← direction (mU6-shTGFBR2→←shPD-1-hU6) and the ←→ direction (shTGFBR2-mU6←→hU6-shPD-1) (FIGS. 1C and 1D). To this end, (1) insertion of a multiple cloning site (MCS), (2) conversion of the mouse U6 promoter of shPD-1 into human U6 promoter, (3) insertion of shTGFBR2 and (4) cloning such as switching positions of shTGFBR2 and shPD-1 are performed.

To insert MCS into the mU6-shPD-1 the 3′ part of pLV-ΔLNGFR_P2A_CD19-CAR_mU6-shPD-1 (plasmid ID #2), primers comprising SEQ ID NOs: 28 and 29 are used to make a PCR product (416 bp) wherein the Hpa1 restriction enzyme recognition site of mU6-shPD-1 3′ is modified into a BsgZ171-Xba1-Nde-1-Bmt1-Spe1 multiple cloning site. Thereafter, the PCR product is treated with BstZ17y and Hpa1 restriction enzymes and plasmid ID #2 is treated with Hpa1 restriction enzyme and CIP, after which blunt end ligation is used to prepare a pLV-ΔLNGFR-P2A-CD19-CAR-mU6-shPD-1_MCS (plasmid ID #4) including the shPD-1-mU6-MCS base sequence (shRNA cassette SEQ ID NO.38).

Thereafter, a plasmid is constructed so that shPD-1 is expressed under the control of the human U6 promoter instead of the mouse U6 promoter. LentiCRISPR V2 plasmid is used with primers comprising SEQ ID Nos: 30 and 31 to obtain a PCR product including the human U6 promoter. The PCR product is treated with Hpa1 and Spe1 restriction enzyme, and then ligated to plasmid ID #4 treated with Hpa1 and Spe1 restriction enzyme to prepare a pLV-ΔLNGFR_P2A_CD19-CAR_hU6-shPD-1_MSC (plasmid ID #5) comprising the hU6-shPD-1 base sequence (SEQ ID NO. 39). The TGFBR2 cassette is inserted into plasmid ID #5 to construct a plasmid which expresses shTGFBR2 and shPD-1 simultaneously.

The PCR product including the shTGFBR2 cassette was obtained using plasmid ID #3 and primers comprising SEQ ID Nos: 32 and 33. After treating the PCR product with Bmt1 and Spe1 restriction enzymes, it is ligated to plasmid ID #5 and treated with Bmt1 and Spe1 restriction enzymes to prepare a pLV-ΔLNGFR_P2A_CD19-CAR mU6-shTGFBR2→←shPD-1-hU6 (plasmid ID #7) comprising a →← direction (mU6-shTGFBR2→←shPD-1-hU6) base sequence.

To build a plasmid wherein two types of shRNa cassettes are arranged in the ←→ directions (shTGFBR2-mU6←→hU6-shPD-1), plasmid ID #7 and primers comprising SEQ ID NOs. 34 and 35 are used to obtain a PCR product. After treating with Spe1 and Hpa1 restriction enzyme, the PCR product is inserted into plasmid ID #4 and treated with the same restriction enzymes to produce pLV-ΔLNGFR-P2A-CD19-CAR-shPD-1-hU6-MCS (plasmid ID #8). Thereafter, plasmid ID #7 and primers comprising SEQ ID NOs. 36 and 37 are used to obtain a PCR product. After treating with Bmt1 and Spe1 restriction enzyme, the PCR product is ligated to plasmid ID #8 and treated with the same restriction enzyme to ultimately prepare a pLV-ΔLNGFR-P2A-CD19-CAR_shTGFBR2-mU6←→hU6-shPD-1 (plasmid ID #10) comprising a ←→ direction (shTGFBR2-mU6←→hU6-shPD-1) base sequence.

The Two-in-One lentiviral vectors wherein two types of shRNA cassette enter in the ←→ or the →← directions produced herein can be used in the production of PD-1 KD modified CAR-T cells in the following examples.

### Example 4: Methods of Production and in Vitro Evaluation of PD-1 KD CAR-T Cells wherein shPD-1, shTGFBR1 and CD19-CAR are Expressed Simultaneously

This example describes the methods of producing and in vitro evaluating dual KD CAR-T cells wherein shPD-1, shTGFBR1 and CD19-CAR are expressed simultaneously.

Plasmid ID #1 (pLV-ΔLNGFR_P2A_CD19-CAR_mU6-shGFP), #6 (pLV-ΔLNGFR_P2A_CD19-CAR_mU6-shTGFBR1→←shPD-1-hU6), #9 (pLV-ΔLNGFR_P2A_CD19-CAR_shTGFBR1-mU6→←hU6-shPD-1), and packaging plasmids pMDL g/p, pRSVrev and pMDG.1 are transfected into HEK293 T cells using lipofectamine, and after 48 hours, the supernatant including the lentivirus is obtained. Using ficoll-paque solution, peripheral blood mononuclear cells (PBMC) are isolated from human blood, and human CD3 and CD28 target antibodies are used to specifically activate the T cells therein. After one to two days following initial activation of the T cells, the cells are transduced using the virus obtained previously from the cell culture. The CAR-T cells are then cultured using AIM-V culture fluid including 5% human plasma and human IL-2. On the sixth day after transduction, the MACSelect LNGFR System (miltenyibiotec, Germany) is used to obtain pure CAR-T cells, and LNGFR target antibody is used to isolate LNGFR+ CAR-T Cells with a flow cytometer. In the following, the cells prepared using plasmid ID #1, #6, #8, and #9 are indicated as ΔLNGFRCART19/shGFP (or shGFP/CART19), ΔLNGFR-CART19/mU6-shTGFBR1→←shPD-1-hU6 (or shPD-1_shTGFBR1/CART19), ΔLNGFR-CART19/shPD-1-hU6, and ΔLNGFR-CART19/shTGFBR1-mU6←→hU6-shPD-1, respectively.

To prepare control CAR-T cells comprising the mU6-shPD-1, mU6-TGFBR1 and shPD-1-hU6 cassettes, respectively, plasmid ID #2 (pLV-ΔLNGFR_P2A_CD19-CAR_mU6-shPD-1), #11 (pLV-ΔLNGFR_P2A_CD19-CAR_mU6-shTGFBR1), and #8 (pLV-ΔLNGFR_P2A_CD19-CAR_shPD-1-hU6_MCS) and packaging plasmids pMDL g/p, pRSVrev and pMDG.1 are transfected into HEK293 T cells using lipofectamine. After 48 hours, the supernatant including the lentivirus is obtained. PBMC are isolated from human blood, and the T cells are activated, transduced and cultured, as described above., On the sixth day after transduction, LNGFR+ CAR-T Cells are isolated as described above. In the following, the cells prepared using plasmid ID #2, #8, #9 and #11 are indicated as ΔLNGFR-CART19/shPD-1(or shPD-1/CART19), ΔLNGFR-CART19/shPD-1-hU6, ΔLNGFR-CART19/shTGFBR1-U6←→hU6-shPD-1, and ΔLNGFR-CART19/shTGFBR1 (or shTGFBR1/CART19), respectively.

To measure the purity of the dual KD CAR-T cells produced herein, flow cytometry is performed using the LNGFR target antibodies for the cells prepared above.

The reduced expression of PD-1 and TGFBR1 in the dual KD CAR-T cells produced herein is measured. The dual KD CAR-T cells are stimulated for three days with human CD3 and CD28 target antibody to induce expression of PD-1 and TGFBR1. Thereafter, the PD-1 and TGFBR1 expression of the dual KD CAR-T cells is analyzed by flow cytometry using CAR, PD-1 and TGFBR1 target antibodies. The impact of dual KD CAR-T cells produced herein on differentiation is measured. To observe the degree of PD-1 and TGFBR1 differentiation of dual KD CAR-T cells, flow cytometry is carried out using CD45RA and CCR7 target antibodies. The transduction efficiency, proliferation ability, and viability of dual KD CAR-T cells into which CD19-CAR vectors comprising shRNA cassettes with different orientations have been introduced are compared. Of the CAR-T cells produced herein, flow cytometry is performed using LNTFR antibody for the cells using plasmids comprising →← direction (mU6-shTGFBR1→←shPD-1-hU6) and ←→ direction (shTGFBR1-mU6←→hU6-shPD-1) cassettes. For cell viability analysis, trypan blue dyeing is performed.

### Example 5: Methods of Production and in Vitro Evaluation of PD-1 KD CAR-T Cells wherein shPD-1, shTGFBR2 and CD19-CAR are Expressed Simultaneously

This example describes the methods of producing and in vitro evaluating dual KD CAR-T cells wherein shPD-1, shTGFBR2 and CD19-CAR are expressed simultaneously.

Plasmid ID #1 (pLV-ΔLNGFR_P2A_CD19-CAR_mU6-shGFP), #7 (pLV-ΔLNGFR_P2A_CD19-CAR_mU6-shTGFBR2→←shPD-1-hU6), #10 (pLV-ΔLNGFR_P2A_CD19-CAR_shTGFBR2-mU6←→hU6-shPD-1), and packaging plasmids pMDL g/p, pRSVrev and pMDG.1 are transfected into HEK293 T cells using lipofectamine, and after 48 hours, the supernatant including the lentivirus is obtained. Using ficoll-paque solution, peripheral blood mononuclear cells (PBMC) are isolated from human blood, and human CD3 and CD28 target antibodies are used to specifically activate the T cells therein. After one to two days following initial activation of the T cells, the cells are transduced using the virus obtained previously from the cell culture. The CAR-T cells are then cultured using AIM-V culture fluid including 5% human plasma and human IL-2. On the sixth day after transduction, the MACSelect LNGFR System (miltenyibiotec, Germany) is used to obtain pure CAR-T cells, and LNGFR target antibody is used to isolate LNGFR+CAR-T Cells with a flow cytometer. In the following, the cells prepared using plasmid ID #1, #7, #8, and #10 are indicated as ΔLNGFRCART19/shGFP (or shGFP/CART19), ΔLNGFR-CART19/mU6-shTGFBR2→←shPD-1-hU6 (or shPD-1_shTGFBR2/CART19), ΔLNGFR_P2A_CD19-CAR_shPD-1-hU6_MCS, and ΔLNGFR-P2A-CD19-CAR_shTGFBR2-mU6←→hU6-shPD-1, respectively (FIGS. 1C and D).

To prepare control CAR-T cells comprising the mU6-shPD-1, mU6-TGFBR2 and shPD-1-hU6 cassettes, or mU6-TGFBR2 and shPD-1-hU6 cassettes respectively, plasmid ID #2 (pLV-ΔLNGFR_P2A_CD19-CAR_mU6-shPD-1), #3 (pLV-ΔLNGFR_P2A_CD19-CAR mU6-shTGFBR2), and #8 (pLV-ΔLNGFR_P2A_CD19-CAR_shPD-1-hU6 MCS) and packaging plasmids pMDL g/p, pRSVrev and pMDG.1 are transfected into HEK293 T cells using lipofectamine. After 48 hours, the supernatant including the lentivirus is obtained. PBMC are isolated from human blood, and the T cells are activated, transduced and cultured, as described above., On the sixth day after transduction, LNGFR+CAR-T Cells are isolated as described above. In the following, the cells prepared using plasmid ID #2, #3, #8, and #10 are indicated as ΔLNGFR-CART19/shPD-1(or shPD-1/CART19), ΔLNGFR-CART19/shTGFBR2 (or shTGFBR2/CART19), ΔLNGFRCART19/shPD-1-hU6, and ΔLNGFR-P2A-CD19-CAR_shTGFBR2-mU6←→hU6-shPD-1, respectively.

To measure the purity of the dual KD CAR-T cells produced herein, flow cytometry is performed using the LNGFR target antibodies for the cells prepared above.

The reduced expression of PD-1 and TGFBR2 in the dual KD CAR-T cells produced herein is measured. The dual KD CAR-T cells are stimulated for three days with human CD3 and CD28 target antibody to induce expression of PD-1 and TGFBR2. Thereafter, the PD-1 and TGFBR2 expression of the dual KD CAR-T cells is analyzed by flow cytometry using CAR, PD-1 and TGFBR2 target antibodies. The impact of dual KD CAR-T cells produced herein on differentiation is measured. To observe the degree of PD-1 and TGFBR2 differentiation of dual KD CAR-T cells, flow cytometry is carried out using CD45RA and CCR7 target antibodies. The transduction efficiency, proliferation ability, and viability of dual KD CAR-T cells into which CD19-CAR vectors comprising shRNA cassettes with different orientations have been introduced is compared. Of the CAR-T cells produced herein, flow cytometry is performed using LNTFR antibody for the cells using plasmids comprising →← direction (mU6-shTGFBR2→←shPD-1-hU6) and ←→ direction (shTGFBR2-mU6←→hU6-shPD-1) cassettes. For cell viability analysis, trypan blue dyeing is performed.

### Example 6: Methods of Production of TGFBR1-Targeting Dual Two-in-One Vectors and Dual KD CAR-T Cells

This example describes the methods of producing TGFBR1-targeting dual Two-in-One vectors and dual KD CAR-T cells.

For generation of dual Two-in-One vectors expressing two shRNAs by different promoters (mU6 and hU6), a BstZ171-Xba1-Nde1-Bmt1-Spe1 multiple cloning site (MCS) is inserted into the pLV-hU6-shPD-1_ΔLNGFR-CD19-BBz vector downstream of hU6 promoter. The second mU6-shRNA cassette fragments are subcloned into the MCS. To limit the blockade of multiple immune checkpoints to CAR-T cells only, a Dual Two-in-One vector is devised to express two shRNAs by mU6 and hU6 Pol III promoters to repress expression of two immune checkpoints of CAR-T cells. To find effective TGFBR1 and PD-1 targeting siRNAs, siRNAs are electroporated to CD3/CD28-stimulated T cells. Two or more effective siRNAs are selected and a Two-in-One vector for TGFBR1-PD-1 KD CAR-T cells are constructed through transformation of 21-mer siRNAs to shRNA format.

The therapeutic potential of the DUAL CAR T cells produced herein is further evaluated in in vivo mice model bearing CD19⁺PDL1⁺ blood tumor and in a solid tumor model in the example below.

### Example 7: Methods of Production of TGFBR2-Targeting Dual Two-in-One Vectors and Dual KD CAR-T Cells

This example describes the methods of producing TGFBR2-targeting dual Two-in-One vectors and dual KD CAR-T cells.

For generation of dual Two-in-One vectors expressing two shRNAs by different promoters (mU6 and hU6), a BstZ171-Xba1-Nde1-Bmt1-Spe1 multiple cloning site (MCS) is inserted into the pLV-hU6-shPD-1_ΔLNGFR-CD19-BBz vector downstream of hU6 promoter. The second mU6-shRNA cassette fragments are subcloned into the MCS. To limit the blockade of multiple immune checkpoints to CAR-T cells only, a Dual Two-in-One vector is devised to express two shRNAs by mU6 and hU6 Pol III promoters to repress expression of two immune checkpoints of CAR-T cells. To find effective TGFBR2 and PD-1 targeting siRNAs, siRNAs are electroporated to CD3/CD28-stimulated T cells. Two or more effective siRNAs are selected and a Two-in-One vector for TGFBR2-PD-1 KD CAR-T cells are constructed through transformation of 21-mer siRNAs to shRNA format.

The therapeutic potential of the DUAL CAR T cells produced herein is further evaluated in in vivo mice model bearing CD19⁺PDL1⁺ blood tumor and in a solid tumor model in the example below.

### Example 8: Methods of Treatment of CD19+ Blood Cancer in Vivo Using Dual KD CAR-T Cells Targeting PD-1 and TGFBR1 or PD-1 and TGFBR2

This example describes the methods of treatment of CD19+ blood cancer in vivo using dual KD CAR-T cells targeting PD-1 and TGFBR1, or PD-1 and TGFBR2.

To establish CD19⁺ blood cancer model, NSG mice (4 to 6 weeks of age) are intravenously injected with 1×10⁶ CD19⁺ NALM-6 leukemia cells that have been engineered to express EGFP-fused firefly luciferase as well as human PDL1 (NALM6-GL-PDL1 cells). CAR-T cells are prepared from the whole blood samples of healthy donors following the procedures described in example 3 above. At day 4 after transduction, CAR⁻T cells are sorted and further expanded for 6 day prior to the injection into mice. Five days after NALM6-GL-PDL1 cell injection, 1×10⁶ CAR-T cells are intravenously infused to the mice. Bioluminescence imaging of NALM6-GL-PDL1 within mice is monitored with Xenogen IVIS Spectrum and the signals are quantified as radiance in the region of interest (photon/sec) using Living Image software (Perkin Elmer).

To examine the effect of cell-intrinsic PD-1 disruption of CAR-T cells on in vivo cytokine production, the peripheral blood of individual mice is obtained at 24 and 72 hours after CAR-T injection. Plasma is harvested from the peripheral blood by centrifugation for 5 minutes at 300×g at room temperature. In vivo cytokine levels are analyzed with Human Th1/Th2 Cytokine Kit (BD Bioscience) following the manufacturer's instructions.

To examine the effect of cell-intrinsic PD-1 disruption of CAR-T cells on in vivo expansion of CAR-T cells, the spleen of individual mice is obtained at 3 or 20 days after CAR-T injection. The percentage of CAR-T cells (Live/Dead⁻CD3⁺) or NALM-6-PDL1(Live/Dead⁻GFP⁺) is evaluated with flow cytometry.

The PD-1 KD CAR-T cells were further used in the methods in the following examples. The antitumor effect of the PD-1_TGFBR1, KD CAR-T cells in a CD19⁺B-ALL model are evaluated. Each CAR-T cells are injected at a single dose of 1×10⁶ day 5 after target cells infusion.

### Example 9: Methods of Treatment in a Solid Tumor Model Using Dual KD CAR-T Cells Targeting PD-1 and TGFBR1 or PD-1 and TGFBR2

This example describes the methods of treatment of cancer in a solid tumor model using dual KD CAR-T cells targeting PD-1 and TGFBR1 or PD-1 and TGFBR2.

To establish the solid tumor model, 4 to 6 week-aged NSG mice are subcutaneously injected with 5×10⁶ IM-9 cells (CD19⁺PD-L1⁺CD155⁺). Once the tumors reach a volume of 150˜250 mm³, CAR-T cells targeting either PD-1 and TGFBR1, or PD-1 and TGFBR2 are intravenously injected at a single dose of 3×10⁶. Tumors are monitored every week by caliper measurement and the volume is estimated by (length×width²)/2.

### Example 10: Generation of Dual Knockdown CAR T Cells

### Materials and Methods

### Cell Lines and Culture Conditions

MDA-MB-231 cells were lentivirally transduced to express Zsgreen to generate MDA-MB-231-Zsgreen. Lenti-X 293T packaging cells were obtained from Takara Bio (Japan). MDA-MB-231-Zsgreen and Lenti-X 293T cells were cultured Dulbecco's modified Eagle medium supplemented with 10% heat-inactivated FBS, 2 mM L-glutamine, 0.1 mM non-essential amino acids, 1 mM sodium pyruvate, and 1% penicillin/streptomycin.

### Plasmid Construction

### EGFR-Specific CAR_shPD-1_shTGFBR2

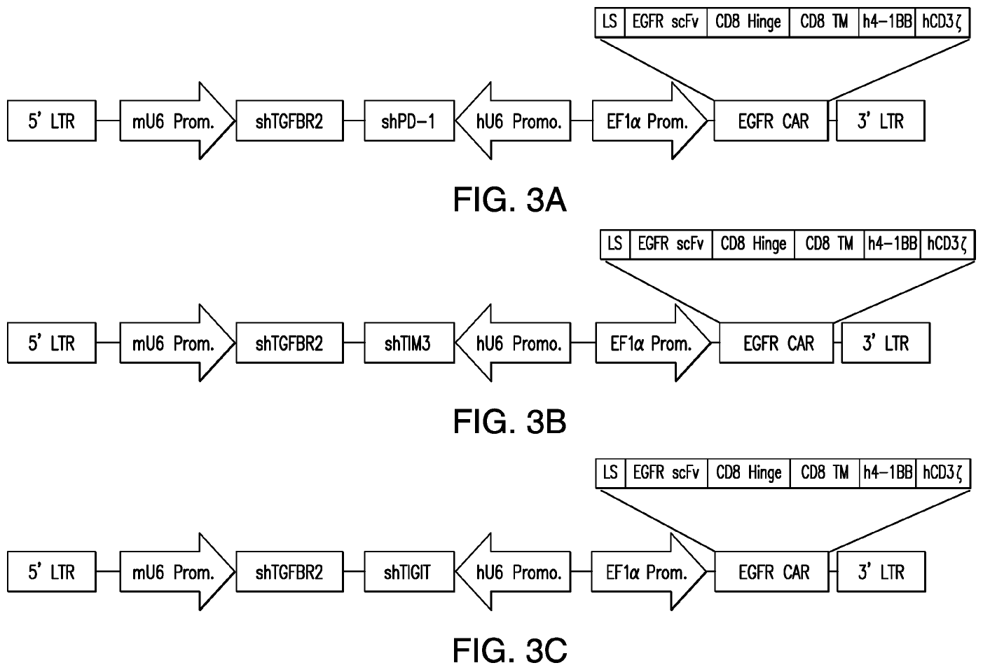

EGFR CAR was designed utilizing the variable light and heavy chains of the Cetuximab antibody sequence been previously described (Hum Vaccin Immunother. 2018; 14(4): 856-863). A DNA encoding the EGFR CAR was synthesized by Bionics (Seoul, Korea) and subcloned into a two-in-one lentiviral vector fused to the 4-1BB intracellular domain sequence and CD3z intracellular domain sequences (see for example, WO 2018/012895, WO 2019/138354). Next, TGFBR2 shRNA cassettes (shRNA cassette SEQ ID #2-7, see Table 2) were synthesized by Bionics (Seoul, Korea) including a 5′ XbaI site and a 3′ SpeI site and moved into pLV-EGFR-CAR_mU6-TIGIT shRNA→←PD-1 shRNA-hU6 (see for example, WO 2018/012895, WO 2019/138354) to replace the mU6-TIGIT shRNA (shRNA cassette SEQ ID #1) to mU6-TGFBR2 shRNA→←PD-1 shRNA-hU6. These plasmids were confirmed by DNA sequencing. The primers are listed in table 1. The resulting construct was designated pLV-EGFR-CAR_hU6-shPD-1_mU6-shTGFBR2 (FIG. 3A).

TABLE 2
shRNA Cassettes

| SEQ ID | shRNA | |
|---|---|---|
| NO | cassette | DNA sequence (5′->3′) |
| 50 | mU6- | TCTAGACATATGGCTAGCGATCCGACGCCGCCATCTCTAGGCC |
| | TIGIT | CGCGCCGGCCCCCTCGCACAGACTTGTGGGAGAAGCTCGGCT |
| | shRNA | ACTCCCCTGCCCCGGTTAATTTGCATATAATATTTCCTAGTAA |
| | | CTATAGAGGCTTAATGTGCGATAAAAGACAGATAATCTGTTCT |
| | | TTTTAATACTAGCTACATTTTACATGATAGGCTTGGATTTCTAT |
| | | AAGAGATACAAATACTAAATTATTATTTTAAAAAACAGCACA |
| | | AAAGGAAACTCACCCTAACTGTAAAGTAATTGTGTGTTTTGAG |
| | | ACTATAAATATCCCTTGGAGAAAAGCCTTGTTTGCTGCATGAC |
| | | TACTTCAATGTTTCAAGAGAACATTGAAGTAGTCATGCAGCTT |
| | | TTTGACTAGT |
| 51 | mU6- | TCTAGACATATGGCTAGCGATCCGACGCCGCCATCTCTAGGCC |
| | TGFBR2 | CGCGCCGGCCCCCTCGCACAGACTTGTGGGAGAAGCTCGGCT |
| | shRNA#1 | ACTCCCCTGCCCCGGTTAATTTGCATATAATATTTCCTAGTAA |
| | | CTATAGAGGCTTAATGTGCGATAAAAGACAGATAATCTGTTCT |
| | | TTTTAATACTAGCTACATTTTACATGATAGGCTTGGATTTCTAT |
| | | AAGAGATACAAATACTAAATTATTATTTTAAAAAACAGCACA |
| | | AAAGGAAACTCACCCTAACTGTAAAGTAATTGTGTGTTTTGAG |
| | | ACTATAAATATCCCTTGGAGAAAAGCCTTGTTTGGAGAAAGA |
| | | ATGACGAGAACATTCAAGAGATGTTCTCGTCATTCTTTCTCCT |
| | | TTTTGACTAGT |
| 52 | mU6- | TCTAGACATATGGCTAGCGATCCGACGCCGCCATCTCTAGGCC |
| | TGFBR2 | CGCGCCGGCCCCCTCGCACAGACTTGTGGGAGAAGCTCGGCT |
| | shRNA#3 | ACTCCCCTGCCCCGGTTAATTTGCATATAATATTTCCTAGTAA |
| | | CTATAGAGGCTTAATGTGCGATAAAAGACAGATAATCTGTTCT |
| | | TTTTAATACTAGCTACATTTTACATGATAGGCTTGGATTTCTAT |
| | | AAGAGATACAAATACTAAATTATTATTTTAAAAAACAGCACA |
| | | AAAGGAAACTCACCCTAACTGTAAAGTAATTGTGTGTTTTGAG |
| | | ACTATAAATATCCCTTGGAGAAAAGCCTTGTTTGGAGGAGAA |
| | | GATTCCTGAAGATTCAAGAGATCTTCAGGAATCTTCTCCTCCT |
| | | TTTTGACTAGT |
| 53 | mU6- | TCTAGACATATGGCTAGCGATCCGACGCCGCCATCTCTAGGCC |
| | TGFBR2 | CGCGCCGGCCCCCTCGCACAGACTTGTGGGAGAAGCTCGGCT |
| | shRNA#4 | ACTCCCCTGCCCCGGTTAATTTGCATATAATATTTCCTAGTAA |
| | | CTATAGAGGCTTAATGTGCGATAAAAGACAGATAATCTGTTCT |
| | | TTTTAATACTAGCTACATTTTACATGATAGGCTTGGATTTCTAT |
| | | AAGAGATACAAATACTAAATTATTATTTTAAAAAACAGCACA |
| | | AAAGGAAACTCACCCTAACTGTAAAGTAATTGTGTGTTTTGAG |
| | | ACTATAAATATCCCTTGGAGAAAAGCCTTGTTTGCATAGAGGC |
| | | GCCTAGAAATTTTCAAGAGAAATTTCTAGGCGCCTCTATGCTT |
| | | TTTGACTAGT |
| 54 | mU6- | TCTAGACATATGGCTAGCGATCCGACGCCGCCATCTCTAGGCC |
| | TGFBR2 | CGCGCCGGCCCCCTCGCACAGACTTGTGGGAGAAGCTCGGCT |
| | shRNA#5 | ACTCCCCTGCCCCGGTTAATTTGCATATAATATTTCCTAGTAA |
| | | CTATAGAGGCTTAATGTGCGATAAAAGACAGATAATCTGTTCT |
| | | TTTTAATACTAGCTACATTTTACATGATAGGCTTGGATTTCTAT |
| | | AAGAGATACAAATACTAAATTATTATTTTAAAAAACAGCACA |
| | | AAAGGAAACTCACCCTAACTGTAAAGTAATTGTGTGTTTTGAG |
| | | ACTATAAATATCCCTTGGAGAAAAGCCTTGTTTGGTTCCTGTG |
| | | TGCCCTTATTTTTCAAGAGAAAATAAGGGCACACAGGAACCT |
| | | TTTTGACTAGT |
| 55 | mU6- | TCTAGACATATGGCTAGCGATCCGACGCCGCCATCTCTAGGCC |
| | TGFBR2 | CGCGCCGGCCCCCTCGCACAGACTTGTGGGAGAAGCTCGGCT |
| | shRNA#7 | ACTCCCCTGCCCCGGTTAATTTGCATATAATATTTCCTAGTAA |
| | | CTATAGAGGCTTAATGTGCGATAAAAGACAGATAATCTGTTCT |
| | | TTTTAATACTAGCTACATTTTACATGATAGGCTTGGATTTCTAT |
| | | AAGAGATACAAATACTAAATTATTATTTTAAAAAACAGCACA |
| | | AAAGGAAACTCACCCTAACTGTAAAGTAATTGTGTGTTTTGAG |
| | | ACTATAAATATCCCTTGGAGAAAAGCCTTGTTTTCCTGCATGA |
| | | GCAACTGCATTCAAGAGATGCAGTTGCTCATGCAGGATTTTTG |
| | | ACTAGT |
| 56 | mU6- | TCTAGACATATGGCTAGCGATCCGACGCCGCCATCTCTAGGCC |
| | TGFBR2 | CGCGCCGGCCCCCTCGCACAGACTTGTGGGAGAAGCTCGGCT |
| | shRNA#8 | ACTCCCCTGCCCCGGTTAATTTGCATATAATATTTCCTAGTAA |
| | | CTATAGAGGCTTAATGTGCGATAAAAGACAGATAATCTGTTCT |
| | | TTTTAATACTAGCTACATTTTACATGATAGGCTTGGATTTCTAT |
| | | AAGAGATACAAATACTAAATTATTATTTTAAAAAACAGCACA |
| | | AAAGGAAACTCACCCTAACTGTAAAGTAATTGTGTGTTTTGAG |
| | | ACTATAAATATCCCTTGGAGAAAAGCCTTGTTTGCTTCTCCAA |
| | | AGTGCATTATTCAAGAGATAATGCACTTTGGAGAAGCTTTTTG |
| | | ACTAGT |

To generate ΔLNGFR, EGFR CAR, and two kinds of shRNA expressing lentiviral plasmid, truncated LNGFR(ΔLNGFR) was amplified from pMACS-ΔLNGFR (Miltenyi Biotec, Germany) using primer #1 and primer #2. EGFR specific CAR (CAR sequence #1) was amplified using primer #3 and primer #4. ΔLNGFR and EGFR specific CAR PCR products were joined by overlap extension PCR using primer #1 and primer #4 and inserted to BamH1 and Sal1 restriction enzyme site of shRNA expressing lentiviral plasmids

TABLE 3
Primers

| SEQ | | |
|---|---|---|
| ID | Primer | |
| NO. | # | DNA sequence (5′→3′) |
| 57 | #1 | GGGGATCCCCCCATCAGTCCGCAAAG |
| 58 | #2 | AAGTTAGTAGCTCCGCTTCCCCACCTCTTGAAGGCTATG |
| | | TAGG |
| 59 | #3 | GGAAGCGGAGCTACTAACTTCAGCCTGCTGAAGCAGGCT |
| | | GGCGACGTGGAGGAGAACCCTGGACCTGCCTTACCAGTG |
| | | ACCGCCTTG |
| 60 | #4 | GTTGATTGTCGACTTAGCGAGGG |

TABLE 4
CAR Sequences

| SEQ | | |
|---|---|---|
| ID NO. | CAR# | Protein sequence (5′→3′) |
| 61 | EGFR- | MALPVTALLLPLALLLHAARPDEVQLVESGGGLVQPGGSLRLS |
| | CAR | CAASGFSFTNYGVHWVRQAPGKGLEWVSVIWSGGNTDYNTS |
| | | VKGRFTISRDNSKNTLYLQMNSLRAEDTAVYYCARALTYYDY |
| | | EFAYWGQGTLVTVSSGGGGSGGGGSGGGGSGGGGSEIVLTQS |
| | | PATLSLSPGERATLSCRASQSIGTNIHWYQQKPGQAPRLLIYYA |
| | | SESISGIPARFSGSGSGTDFTLTISSLEPEDFAVYYCQQNNNWPT |
| | | TFGQGTKLEIKGSLETTTPAPRPPTPAPTIASQPLSLRPEACRPA |
| | | AGGAVHTRGLDFACDIYIWAPLAGTCGVLLLSLVITLYCKRGR |
| | | KKLLYIFKQPFMRPVQTTQEEDGCSCRFPEEEEGGCELRVKFSR |
| | | SADAPAYQQGQNQLYNELNLGRREEYDVLDKRRGRDPEMGG |
| | | KPRRKNPQEGLYNELQKDKMAEAYSEIGMKGERRRGKGHDG |
| | | LYQGLSTATKDTYDALHMQALPPR* |
| 62 | Cetuximab | DEVQLVESGGGLVQPGGSLRLSCAASGFSFTNYGVHWVRQAP |
| | scFV | GKGLEWVSVIWSGGNTDYNTSVKGRFTISRDNSKNTLYLQMN |
| | | SLRAEDTAVYYCARALTYYDYEFAYWGQGTLVTVSSGGGGS |
| | | GGGGSGGGGSGGGGSEIVLTQSPATLSLSPGERATLSCRASQSI |
| | | GTNIHWYQQKPGQAPRLLIYYASESISGIPARFSGSGSGTDFTLT |
| | | ISSLEPEDFAVYYCQQNNNWPTTFGQGTKLEIKGSLE |

### Generation of Human CAR T Cells

Three days before transduction, Lenti-X 293T cells were seeded at 4×10⁶ cells per 100 mm dish. Twenty-four hours later, pLV encoding shRNA and CAR (7.5 μg), pMDG.1 encoding VSV-G envelope (4.5 μg), pRSV-Rev encoding Rev (6 μg), and pMDLg/pRRE encoding Gag/Pol (6 μg) vector were transfected using Lipofectamine 2000. Forty-eight hours later, supernatants were collected, and cell debris was removed by centrifugation at 1600 rpm for 5 min. Peripheral blood mononuclear cells (PBMCs) were collected from whole blood samples of healthy donors using SepMate tubes (STEMCELL Technologies, Canada) in accordance with the manufacturer's instructions. PBMCs were stimulated with 4 μg/mL of plate-bound anti-CD3 antibody (clone OKT3; Bio X Cell), 2 μg/mL of soluble anti-CD28 antibody (clone CD28.2; Bio X Cell), and 300 IU/mL human recombinant IL-2 (BMI KOREA, Republic of Korea) in the complete T-cell medium containing 90% RPMI 1640 (Gibco, USA) supplemented with 10% heat-inactivated FBS (Gibco, USA), 0.1 mM non-essential amino acids (Gibco, USA), 2 mM GlutaMAX (Gibco, USA), 1mM sodium pyruvate (Gibco, USA) and 0.05 mM 2-mercaptoethanol (Gibco, USA) with 300 IU/mL rhIL-2. Two days after CD3/CD28 stimulation, activated T cells were transduced with lentiviral supernatants with protamine at a 10 μg/ml, followed by centrifugation for 90 min at 1000×g at 32° C. Twenty-four hours later, supernatants were removed, transduced T cells were expanded in fresh complete T-cell medium with 300 IU/mL rhlL-2. The fresh medium was added every 2 days. For isolate CAR transduced T cells, T cells were isolated using the human CD271 MicroBead kit (Cat #130-099-023, Miltenyi Biotec) following the manufacturer's instructions. CAR⁺ ΔLNGFR⁺T cells were maintained in a complete T-cell medium with 300 IU/mL rhIL-2 for 6 days.

### FACS Analysis

All FACS analyses were performed on the LSRFortessa™ X-20 cytometer (BD, USA) and evaluated with FlowJo software (Tree Star, USA). Cell surface staining for flow cytometry was performed by pelleting and resuspending in 100 μL FACS buffer (2% FBS and 2 mM EDTA in PBS) with antibodies for 15 min at RT in the dark. Fifteen minutes later, cells were washed with 2 mL FACS buffer, resuspended, and analyzed. Four days after transduction, the percentage of transduced T cells was evaluated by ΔLNGFR and EGFR-CAR expression using APC conjugated anti-LNGFR antibody (Cat #130-113-418, Miltenyi Biotec, Germany) and Biotinylated human EGFR protein (Cat #EGR-H82E3, ACRO Biosystems, USA).

To determine the PD-1, TIM-3, TIGIT protein level of CART cells, CART cells were stimulated with T Cell TransAct™ (Cat #130-111-160, Miltenyi Biotec, Germany) After 48 hours, PD-1 expression of CART cells was assessed using APC conjugated anti-LNGFR antibody (Cat #130-113-418, Miltenyi Biotec, Germany), PE-conjugated anti-PD-1 antibody (Cat #12-2799-42, Thermo, USA), PE-conjugated anti-TIM-3 antibody (Cat #FAB2365P, R&D systems, USA), PE-conjugated anti-TIGIT antibody (Cat #12-9500-42, Thermo, USA). To evaluate the expression level of EGFR or inhibitory ligands in MDA-MB-231 cells, APC-conjugated anti-human PDL1 (Cat #329707, Biolegend, USA), PE-conjugated anti-CD155 (Cat #337609, Biolegend, USA), PE-conjugated anti-CD112 (Cat #337409, Biolegend, USA), PE-conjugated anti-CD80 (Cat #305208, Biolegend, USA), or PE-conjugated anti-CD86 (Cat #562432, Biolegend, USA) or PE-conjugated anti-human EGFR (Cat #555997, BD, USA) antibodies were used.

To detect intracellular galectin-9, MDA-MB-231 cells were fixed/permeabilized using 200 μL BD Cytofix/Cytoperm solution for 20 min at 37° C. Twenty minutes later, cells were washed and stained with PerCP-Cy5.5-conjugated anti-Galectin-9 (Cat #348910, Biolegend, USA) antibody.

### Immunoblotting

CAR T cells were stimulated with T Cell TransAct™ (Cat #130-111-160, Miltenyi Biotec, Germany) After 48 hours, whole-cell protein lysates were obtained in denaturing buffer (50 mM Tris-HCl, pH 7.5, 150 mM NaCl, 1% NP-40, 0.5% deoxycholic acid, 0.1% SDS, 1 mM PMSF) with Halt™ protease & phosphatase inhibitor cocktail (Cat #1861280, Thermo, USA). Protein concentrations were estimated by BCA assay (Cat #23227, Thermo USA). Immunoblotting was performed by loading 15 μg of protein onto 4-12% Bis-TrisPAGE gels followed by transfer to PVDF membranes. Signals were detected by Enhanced Peroxidase Detection kit (Cat #EBP-1071, ELPISBIO, Korea) with the ChemiDoc XRS+ System. TGFBR2 specific antibody was purchased from Elabscience (Cat #E-AB-33088). SMAD2(D43B4) and pSMAD2(138D4) specific antibodies were purchased from Cell Signaling.

### Incucyte-Based Cytotoxicity

1×10⁵ MDA-MB-231-Zsgreen cells were seeded in 96-well flat-bottom plates. After 24 hr, CAR T cells were added at a 1:1 to 0.1:1 E:T ratios in the absence or presence of 5 ng/mL recombinant human TGF-β1 (Cat #240-B-010, R&D systems). Triplicate wells were plated for each CART group. GFP fluorescent intensity per well was detected every 2 hr using the IncuCyte S3 Live-Cell analysis system (Sartorius, Germany) and collected at each time point. Total integrated GFP intensity per well was as a quantitative measure of viable target cells. Values of total integrated GFP intensity were normalized to the starting point.

### Multi-Round Antigen Stimulation Assay

1×10⁵ MDA-MB-231-Zsgreen cells were seeded in 96-well flat-bottom plates. After 24 hr, 1×10⁵ CAR T cells were incubated in the absence or presence of 5 ng/mL recombinant human TGF-β1. The new 2×10⁵ MDA-MB-231-Zsgreen cells were added every 2 days. The final concentration of TGF-β1 was maintained at 5 ng/mL in the addition group. 8 days after the addition of CAR T cells, Total live cells were counted using the countess II Automated Cell Counter (ThermoFisher, USA) and the percentage of LNGFR⁺ or Zsgreen⁺ cells was determined by flow cytometry. Fold change of CAR-T cells was calculated using a ratio [number of live CAR T cells at day 8/numbers of live CAR-T cells at day 8]. Relative fold change of CAR T cells was calculated using a ratio [Fold change of TGF-β1 treated CAR T cells /Fold change of untreated CAR T cells].

### Statistics

Statistically significant differences for comparisons between two samples were assessed using a two-tailed paired or unpaired t-test. Comparisons between more than two groups were carried out by an ANOVA with Tukey's multiple comparisons test. GraphPad Prism 6.0 was used for statistical calculations. p<0.05 was considered statistically significant.

### Results

### Generation of Dual Knockdown CAR T Cells

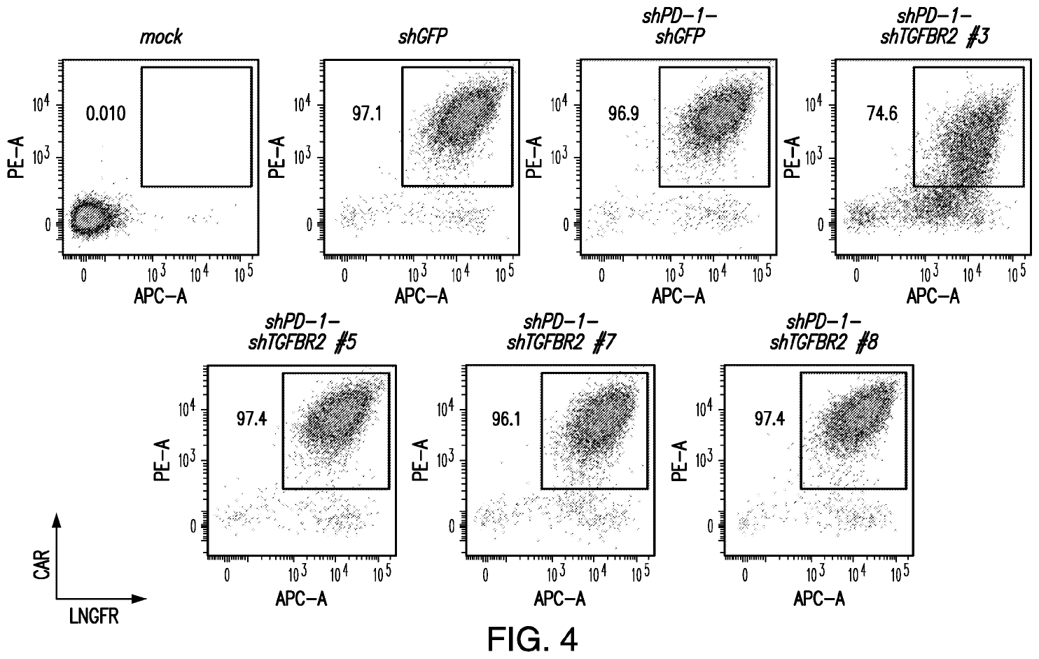

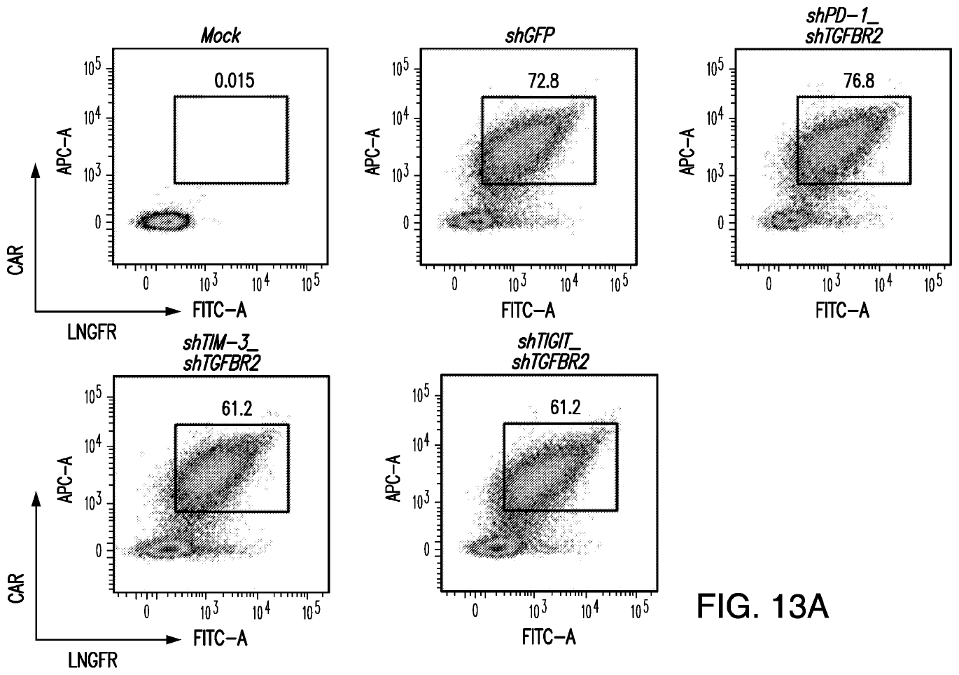

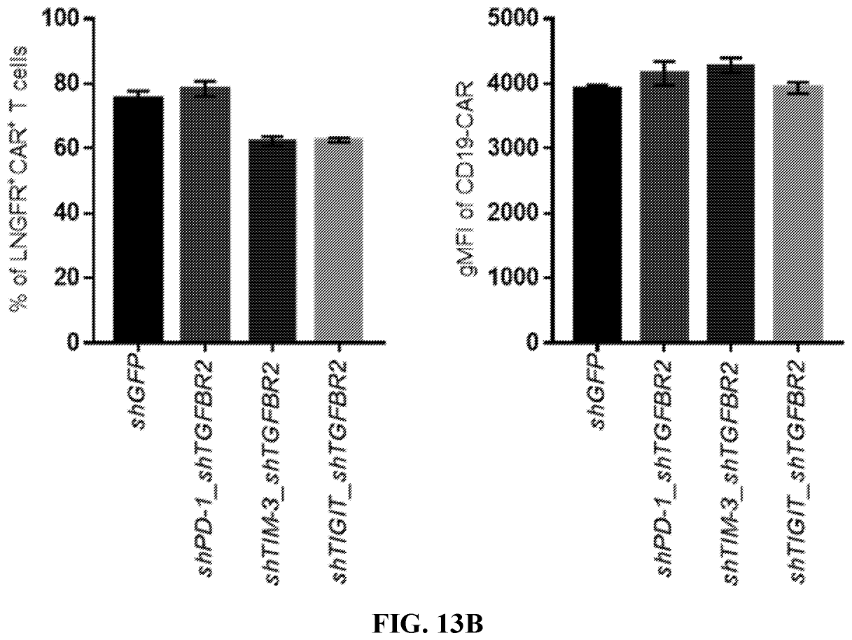

FIG. 4 shows that T cells could efficiently be transduced with a CAR in the presence of shRNAs targeting both PD-1 and TGFBR2. Most shRNA constructs against TGFBR2 showed no effect on transduction efficiency of the CAR. Similarly, FIGS. 13A and 13B show that T cells could efficiently be transduced with CARs in the presence of dual knockdown of TIM3 and TGFBR2 (shRNA #5) or TIGIT and TGFBR2 (shRNA #5).

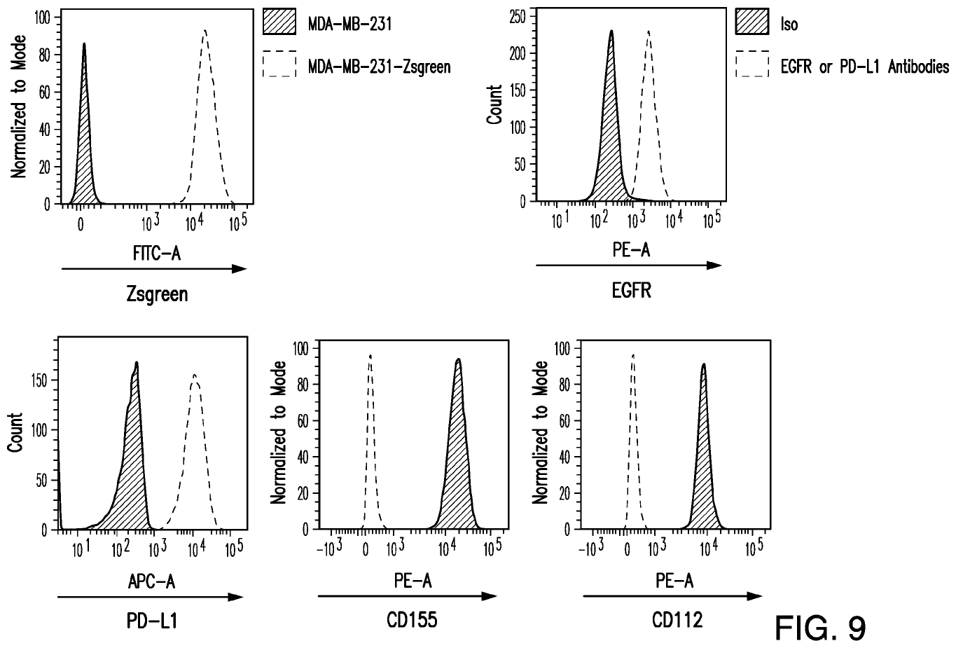

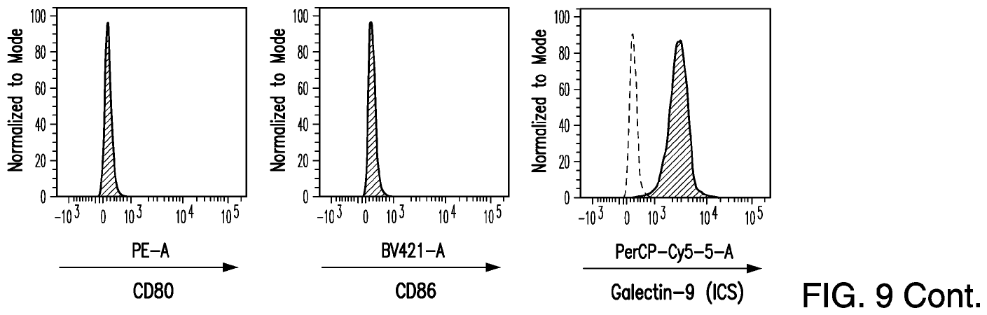

FIG. 9 shows that EGFR (the target of the CAR T cells used in the experiments described in this Example), PDL1 (ligand of PD-1), CD155 and CD112 (both ligands of TIGIT), as well as CD80 and CD86 (both ligands of CTLA-4) and Galectin-9 (ligand of TIM-3) are expressed in the MDA-MB-231-Zsgreen cell line used herein.

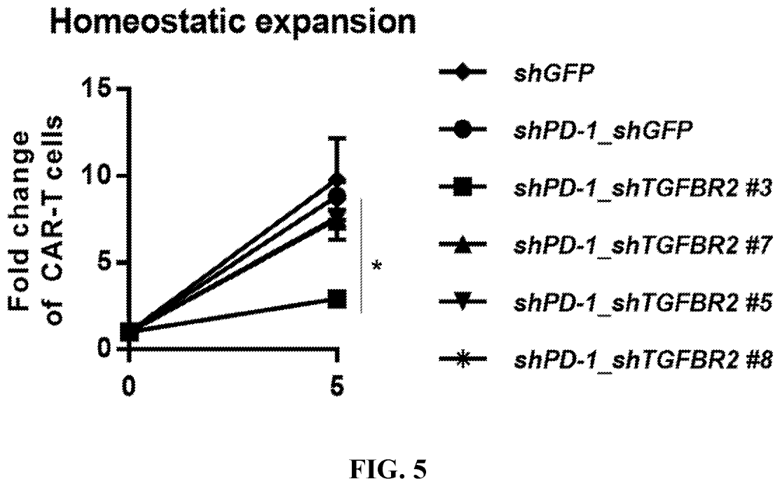

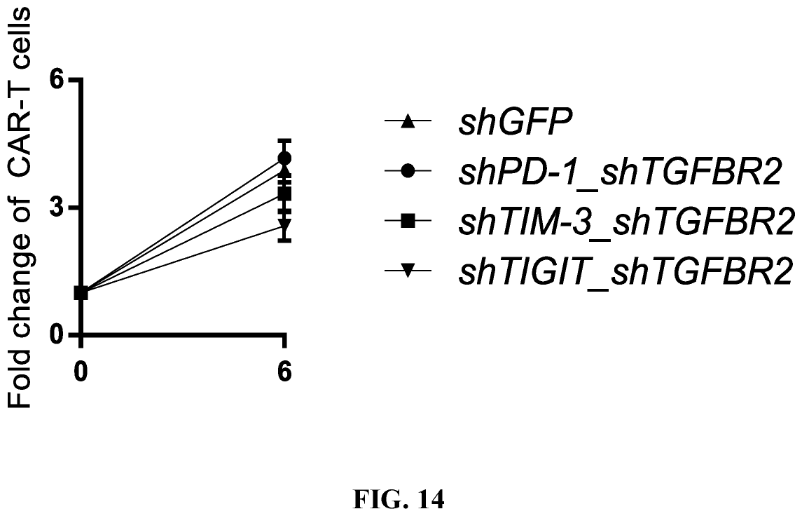

FIG. 5 and FIG. 14 show that dual knockdown CAR T cells transfected with shRNA constructs targeting shPD-1 and TGFBR2 could be expanded in vitro. Most of the shRNA constructs targeting TGFBR2 tested in combination with shRNA construct targeting shPD-1 did not have a significant effect on the homeostatic expansion of CAR T cells compared to the effect of shRNA targeting PD-1 in combination with shRNA construct targeting GFP. See FIG. 5. Similarly, there was no significant decrease in expansion of CAR T cells in the presence of shTIM3 and shTGFRB2 #5 nor in the presence of shTIGIT and shTGFBR2 #5. See FIG. 14.

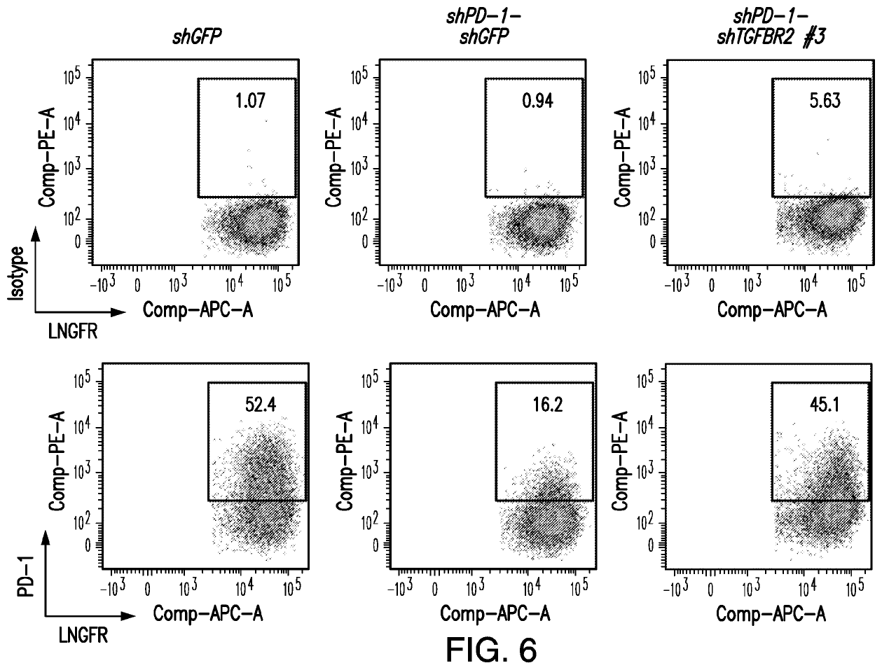

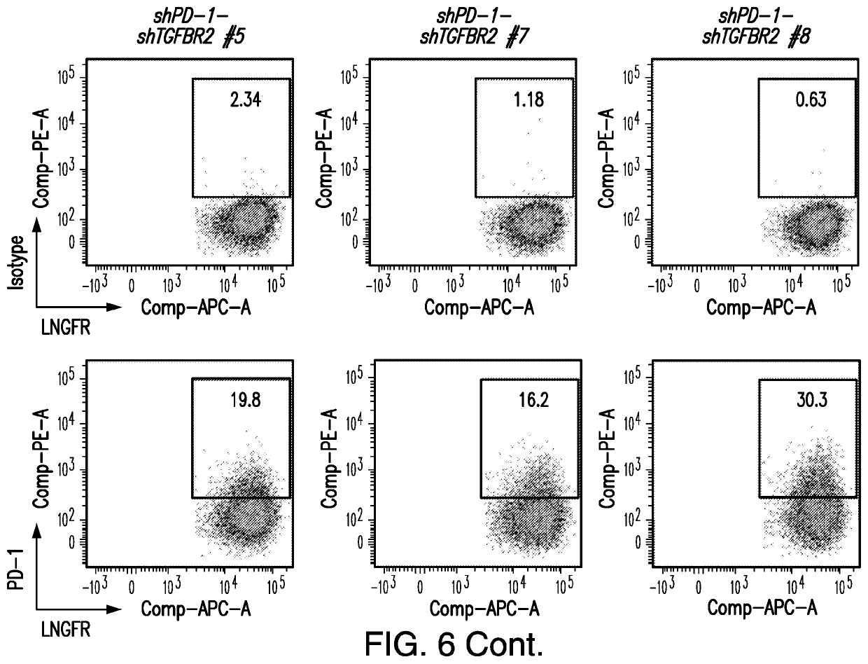

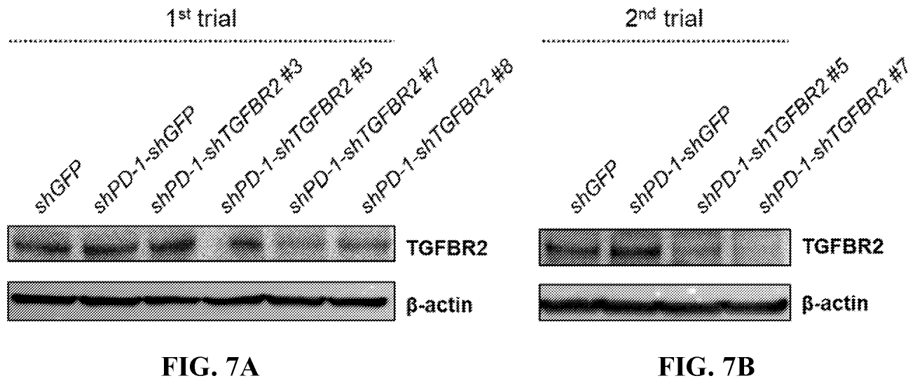

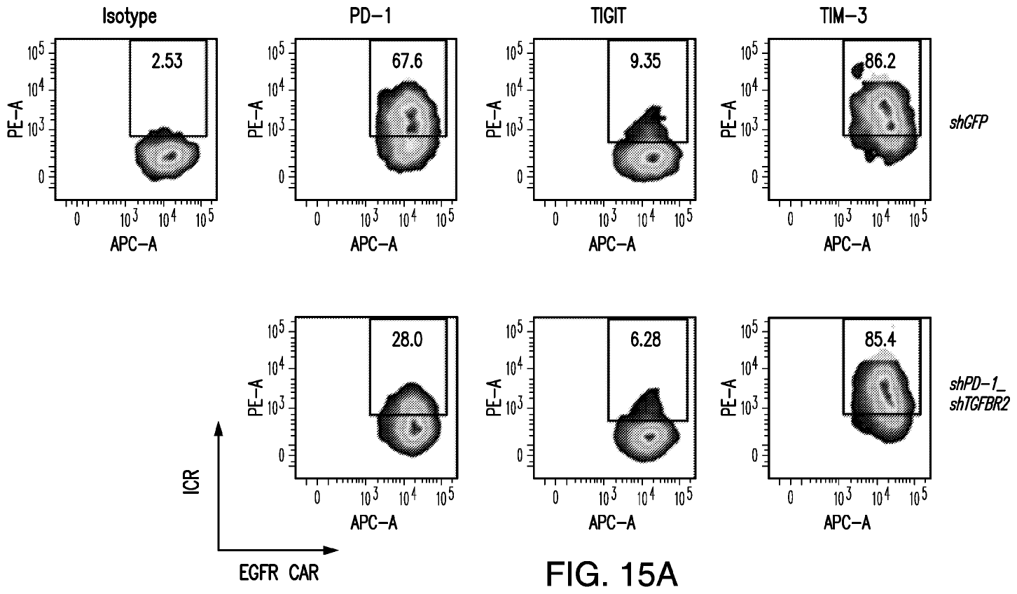

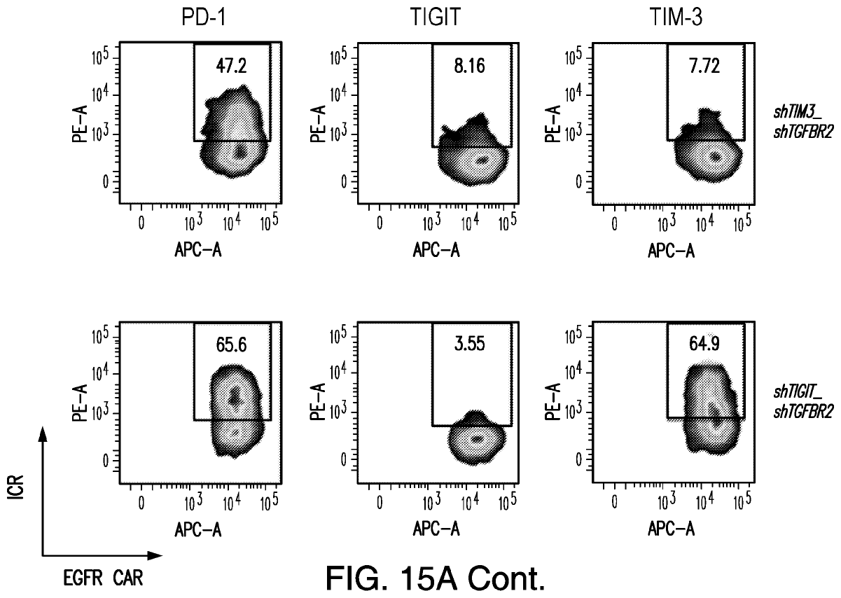

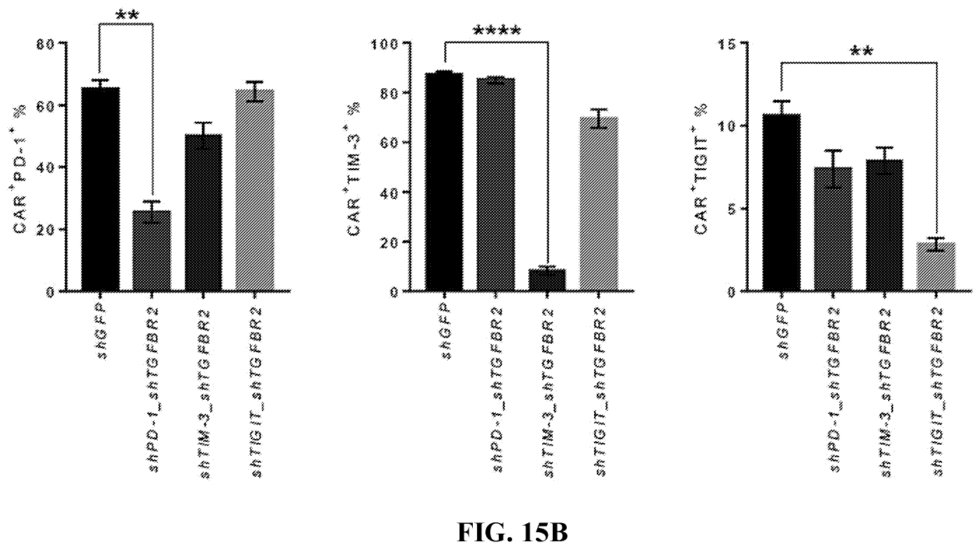

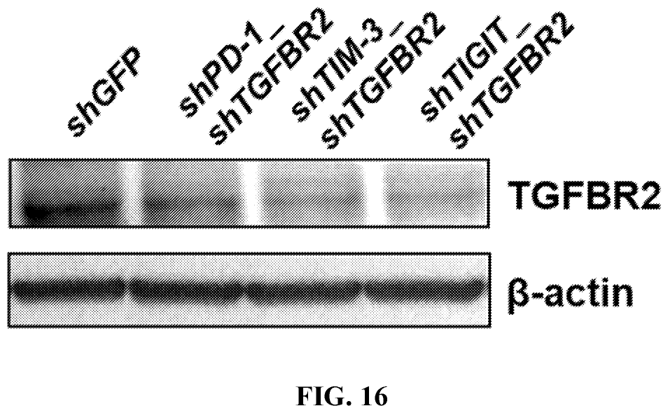

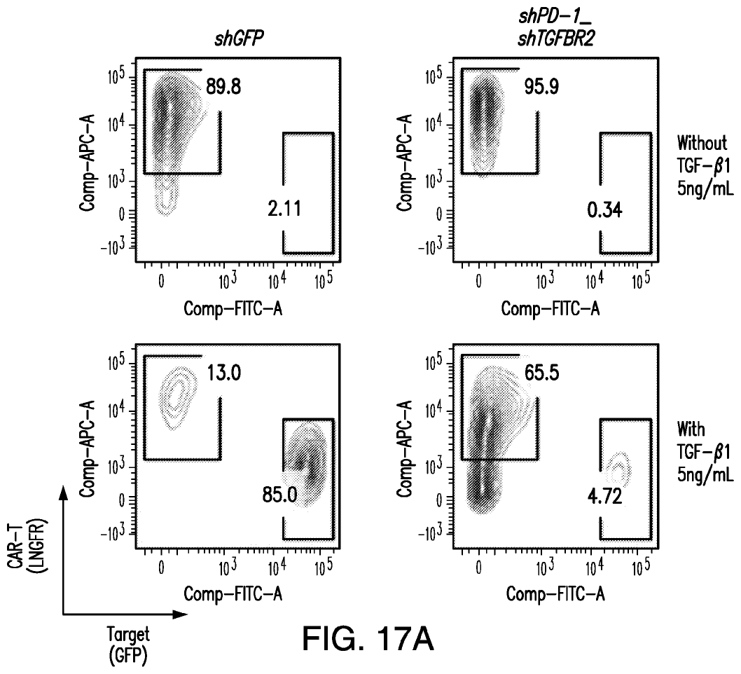

Dual knockdown of PD-1 and TGFBR2 could be achieved in CAR T cells as shown in FIG. 6. The knockdown of shPD-1 achieved with an shRNA targeting PD-1 in combination with an shRNA targeting GFP was not substantially different to the knockdown of PD-1 achieved with an shRNA targeting PD-1 in combination with an shRNA construct targeting TGFBR2 for two out of the four shTGFBR2 constructs tested (shTGFBR2 #5 and #7). Further, FIGS. 7A and 7B show that protein levels of TGFBR2 were decreased in CAR T cells transfected with shPD-1 and shTGFBR2 #5, #7 or #8. Knockdown of TIM3 and TIGIT could also be achieved in CAR T cells expressing shTIM3 and shTGFBR2 (shRNA #5) or shTIGIT and shTGFBR2 (shRNA #5), respectively. See FIGS. 15A and 15B. FIG. 16 shows that the presence of shPD-1, shTIM3 or shTIGIT did not substantially affect the knockdown efficiency of shTGFBR2 #5.

### Functional Analysis of Dual Knockdown CAR T Cells

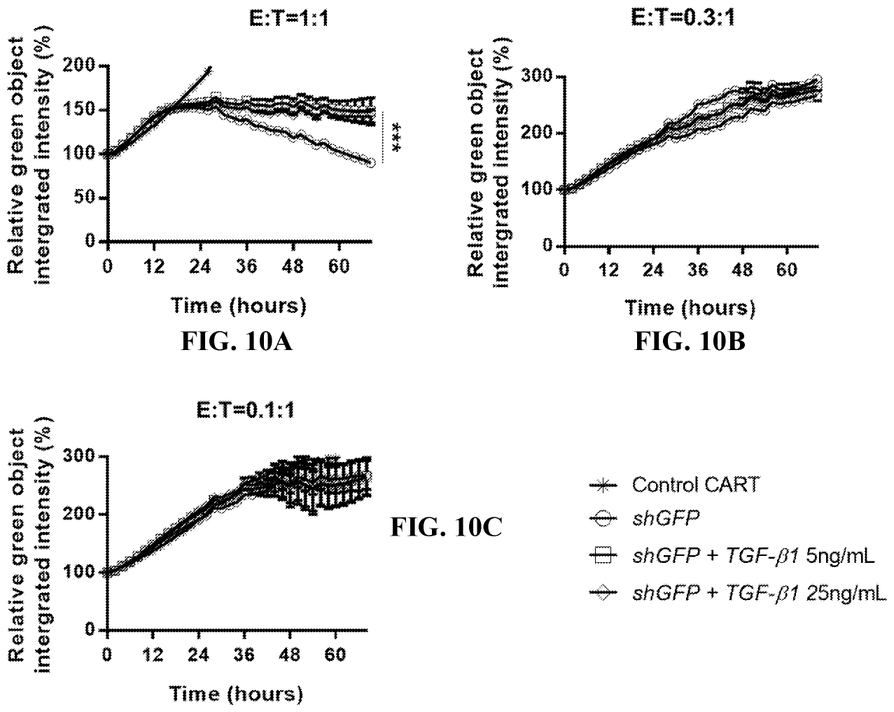

To determine the optimal concentration of TGF-β1 for in vitro analysis, the efficiency of the dual knockdown CAR T cells was assessed by co-culturing the CAR T cells with GFP-expressing target cells (MDA-MB-231-Zsgreen) in the presence of varying concentrations of TGF-β1 and the decrease in GFP intensity was measured by incucyte. FIGS. 10A-10C show that there was no significant difference in the effect of the two TGF-β1 concentrations tested. Therefore, 5 ng/mL TGF-β1 was used in subsequent experiments.

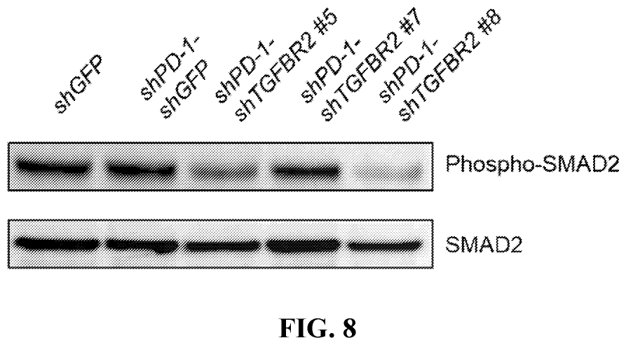

The effect of dual shRNA knockdown of PD-1 and TGFBR2 was assessed using Western Blotting. FIG. 8 shows that phosphorylation of SMAD2 decreased in CAR T cells transfected with shPD-1 and shTGFBR2 #8. SMAD2 is a major signal transduction pathway for TGFβ receptor signaling. Thus, dual knockdown of PD-1 and TGFBR2 decreases TGFβ receptor signaling.

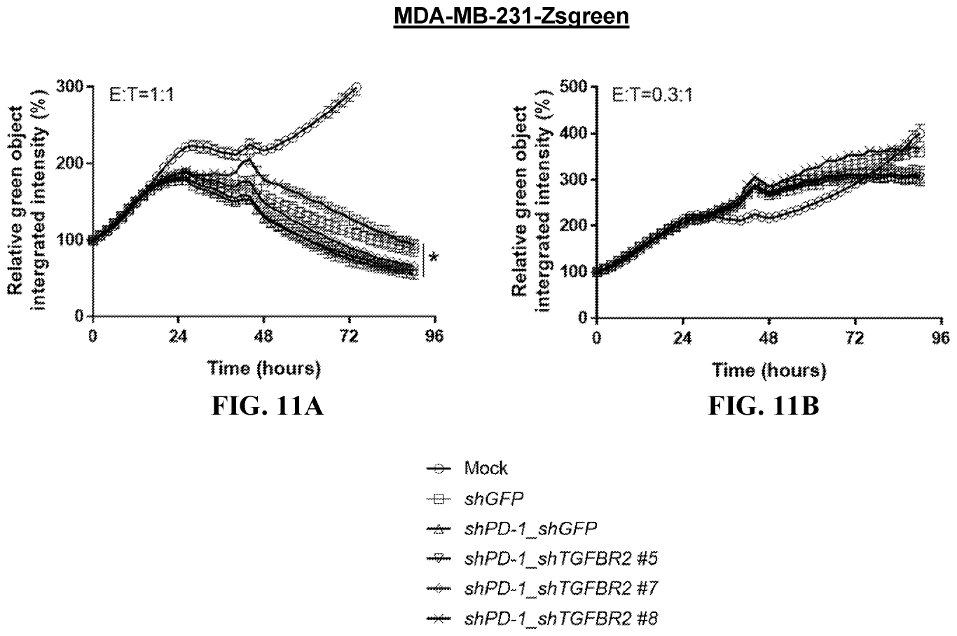

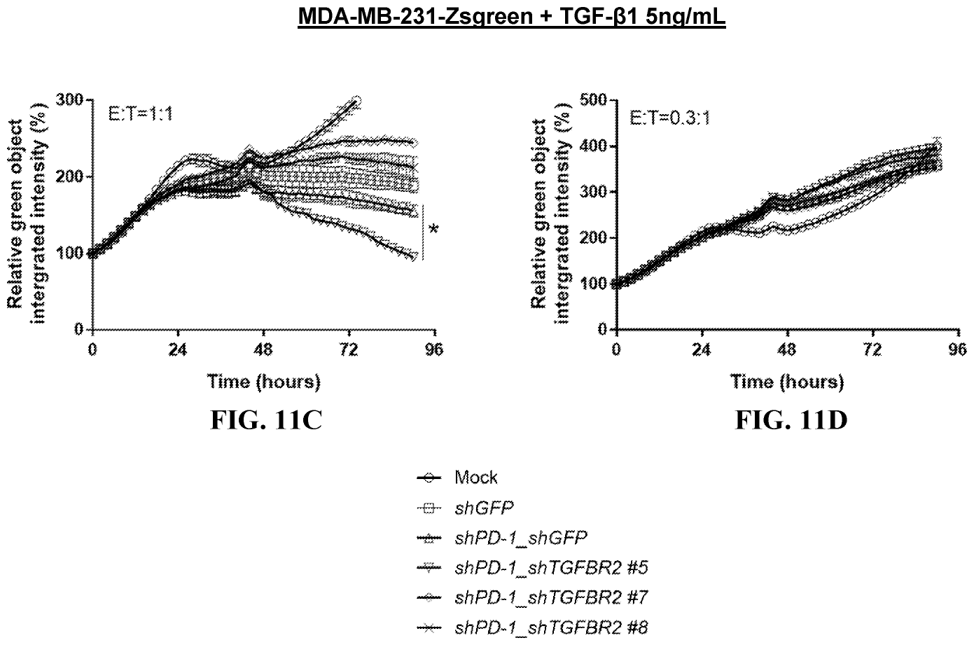

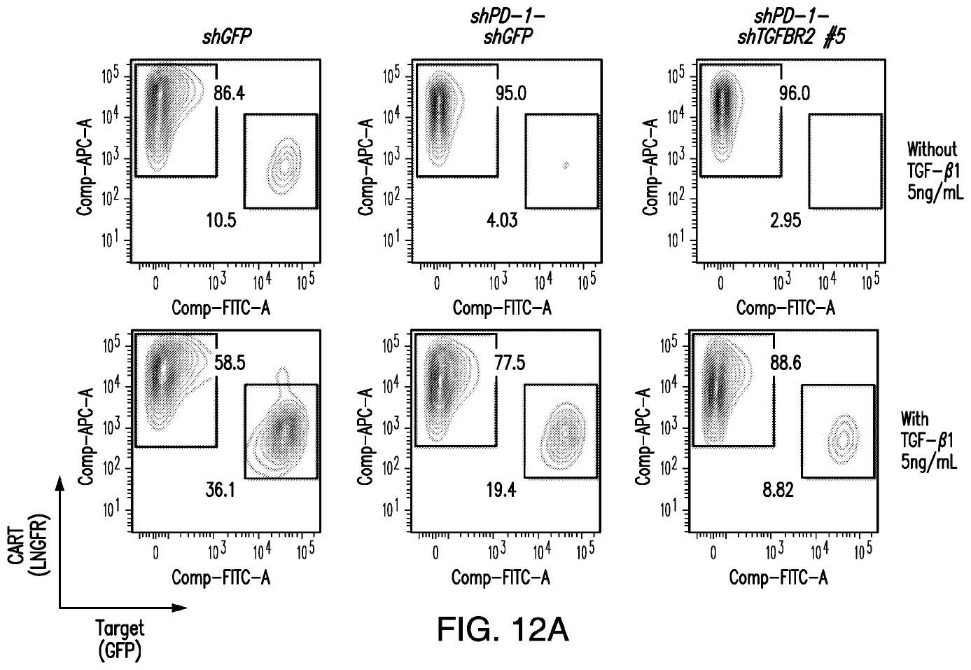

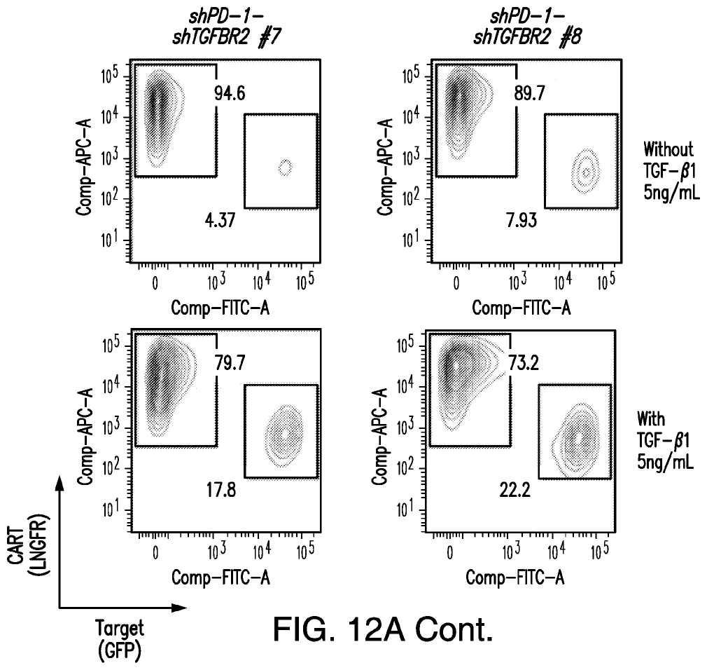

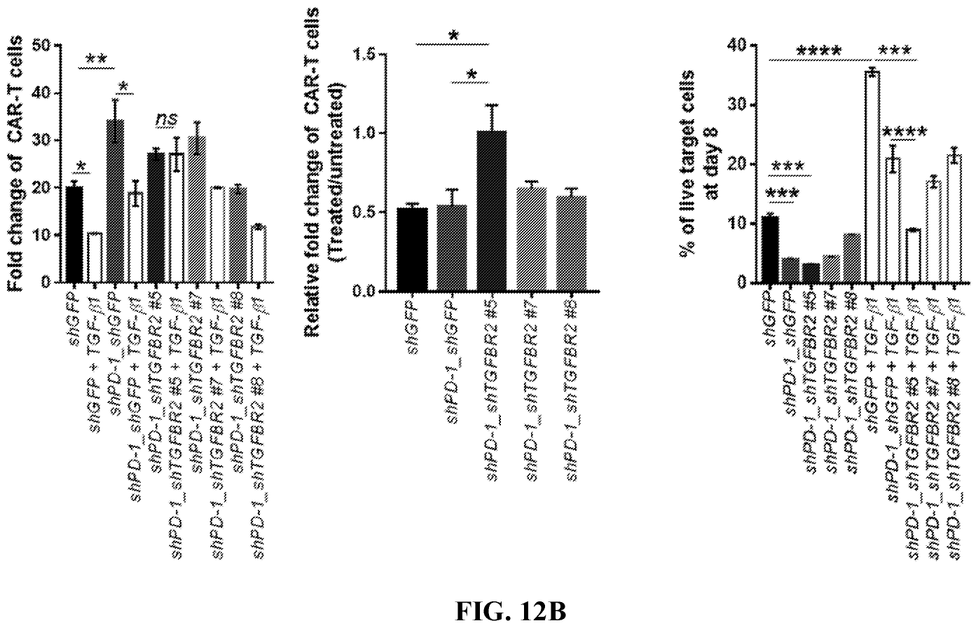

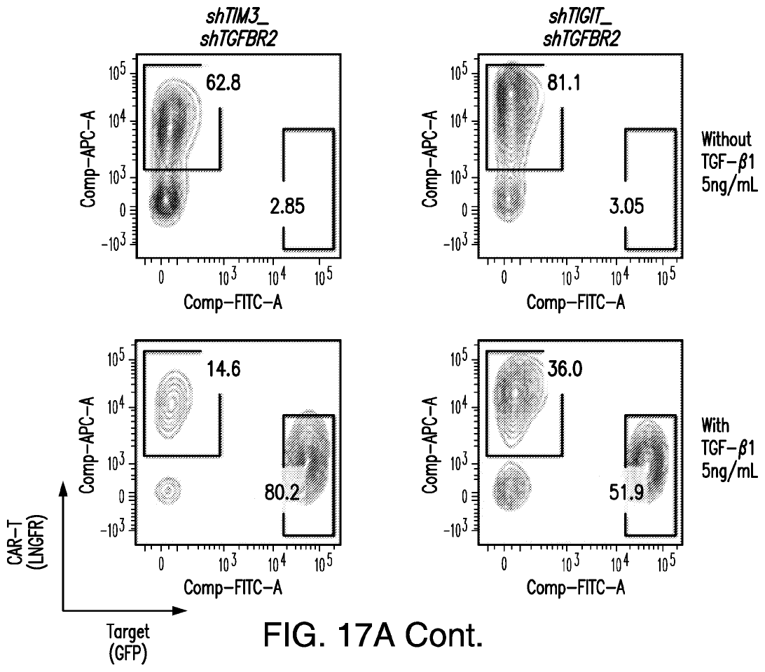

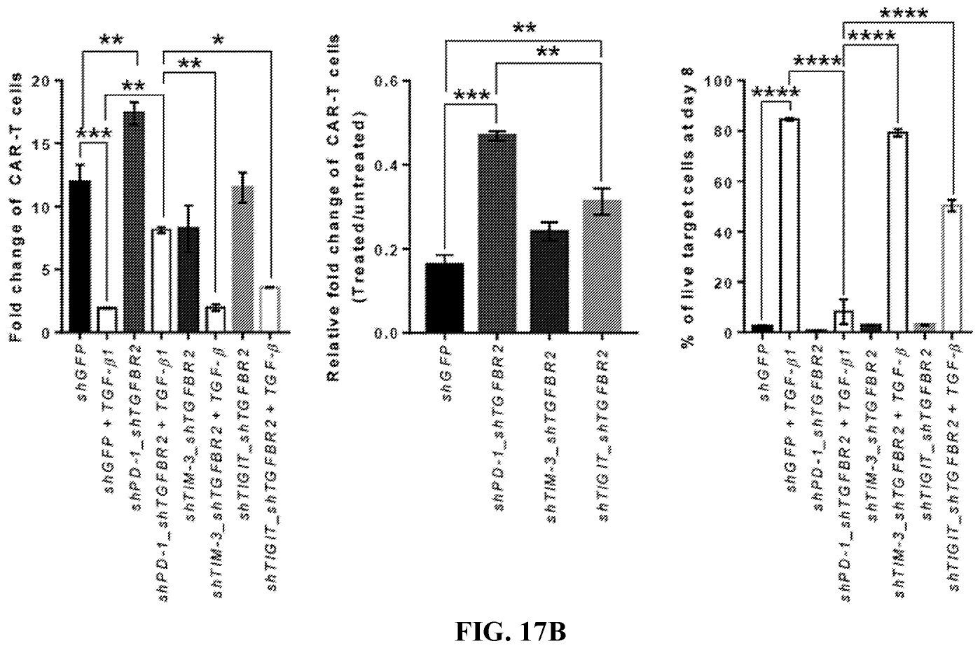

As shown in FIGS. 11A-11D, dual knock of PD-1 and TGFBR2 in CART cells does not significantly decrease the cytotoxicity of the CAR T cells in the absence of TGF-β1. FIG. 11C shows that the combination of shPD-1 and shTGFBR2 #5 significantly increased the cytotoxicity of the dual knockdown CAR T cell in the presence of TGF-β1 at an effector to target cell ratio of 1:1. Data shown in FIGS. 12A and 12B show that number of CART cells increased with dual knockdown of PD-1 and TGFBR2 using shTGFBR2 #5 with repeat stimulation by TGFβ1 for eight days, but remained unchanged relative to control (without TGFβ1) for single knockdown of PD-1 or dual knockdown using the other two shTGFBR2 constructs. Meanwhile, the percentage of viable target cells was higher in the presence of TGF-β1 compared to control without TGF-β1. The number of viable target cells significantly decreased in the presence of dual knockdown CAR T cells transfected with shPD-1 and shTGFBR2 #5, and this effect was seen both in the presence and absence of TGF-β1. FIG. 17 shows that, surprisingly, shTGFBR2 in combination with shTIM3 or TIGIT decreased CAR T cell viability compared to the combination of shTGFBR2 (shRNA #5) and shPD-1.

1. A vector, comprising:
a base sequence encoding two types of short hairpin RNA (shRNA) which inhibit the expression of at least two genes that weaken the function of immune cells, and
a base sequence encoding a chimeric antigen receptor (CAR) or a monoclonal T cell receptor (mTCR).

2. The vector according to claim 1, wherein expression of the two types of shRNA is characterized in that they are regulated by two different promoters.

3. The vector according to claim 2, wherein the two promoters are RNA polymerase III promoters.

4. The vector according to claim 2, wherein the two promoters are U6 promoters derived from different species.

5. The vector according to claim 2, wherein the two promoters are oriented in different directions from each other on the vector.

6. The vector according to claim 1, wherein at least one or two of the genes weakening the function of immune cells are PD-1, TGFBR1, or TGFBR2.

7. The vector according to claim 1, wherein the genes weakening the function of immune cells are immune checkpoint receptors or ligands.

8. The vector according to claim 7, wherein the immune checkpoint receptor is PD1.

9. The vector according to claim 1, wherein, of the two types of shRNA, one shRNA targets PD-1 and the second shRNA targets TGFBR1.

10. The vector according to claim 1, wherein, of the two types of shRNA, one shRNA targets PD-1 and the second shRNA targets TGFBR2.

11. The vector according to claim 1, wherein, of the two types of shRNA, the base sequence encoding one shRNA comprises a sequence selected from a group consisting of SEQ ID NOs: 43-47, and the base sequence encoding the second shRNA comprises a different sequence selected from the group consisting of SEQ ID Nos: 13-23.

12. The vector according to claim 1, wherein, of the two types of shRNA, the base sequence encoding one shRNA comprises a sequence selected from a group consisting of SEQ ID NOs: 1-12, and the base sequence encoding the second shRNA comprises a different sequence selected from the group consisting of SEQ ID Nos: 13-23.

13. The vector according to claim 1, wherein the target of the CAR or mTCR is a human tumor antigen that exhibits increased expression in a cancer cell, cancer tissue, and/or tumor microenvironment, or is a mutated form of antigen found in a cancer cell, cancer tissue and/or tumor microenvironment.

14. The vector according to claim 1, wherein the vector is a plasmid vector, a lentivirus vector, an adenovirus vector, an adeno-associated vector or a retrovirus vector.

15. An immune cell comprising the vector according to claim 1, wherein expression of at least one or two of the genes is reduced to 40% or less than that of expression in the absence of the shRNAs.

16. The immune cell according to claim 15, wherein the immune cell is a human-derived T cell or natural killer (NK) cell.

17. A pharmaceutical composition comprising the immune cell according to any one of claim 1-16.

18. The pharmaceutical composition according to claim 17, for treatment of a patient in need of immune therapy, wherein the immune cell is originally obtained from the patient.

19. The pharmaceutical composition according to claim 18, wherein the patient has a tumor or cancer in which the target, and/or an increase or variation in levels of the target, of the CAR or mTCR expressed in the immune cell is detected.

20. An immune cell comprising a genetically engineered antigen receptor that specifically binds to a target antigen and a genetic disruption agent reducing or capable of reducing the expression in the immune cell of at least two genes that weaken the function of the immune cell.

21. The immune cell of claim 20, wherein the genetically engineered antigen receptor is a chimeric antigen receptor (CAR) or a T cell receptor (TCR).

22. The immune cell of claim 21, wherein the genetically engineered antigen receptor is a CAR.

23. The immune cell of claim 22, wherein the CAR comprises an extracellular antigen recognition domain, a transmembrane domain, and an intracellular signal transduction domain.

24. The immune cell of claim 23, wherein the extracellular antigen recognition domain of the CAR specifically binds to the target antigen.

25. The immune cell of claim 23, wherein the intracellular signal transduction domain of the CAR comprises an intracellular domain of a CD3 zeta (CD3ζ) chain.

26. The immune cell of claim 25, wherein the intracellular signal transduction domain of the CAR further comprises a costimulatory molecule.

27. The immune cell of claim 26, wherein the costimulatory molecule is selected from the group consisting of ICOS, 0X40, CD137 (4-1BB), CD27, and CD28.

28. The immune cell of claim 27, wherein the costimulatory molecule is CD137 (4-1BB).

29. The immune cell of claim 27, wherein the costimulatory molecule is CD28.

30. The immune cell of claim 21, wherein the genetically engineered antigen receptor is a TCR.

31. The immune cell of claim 30, wherein the TCR is a monoclonal TCR (mTCR).

32. The immune cell of claim 30 or 31, wherein the target antigen is expressed in or on the surface of a cancer cell, a cancer tissue, and/or a tumor microenvironment.

33. The immune cell of claim 32, wherein the target antigen is selected from the group consisting of:
5T4 (Trophoblast glycoprotein), 707-AP, 9D7, AFP (a-fetoprotein), AlbZIP (androgen-induced bZIP), HPG1 (human prostate specific gene-1), α5β1-Integrin, α5β6-Integrin, α-methylacyl-coenzyme A racemase, ART-4 (ADPribosyltransferase-4), B7H4 (v-set domain-containing T-cell activation inhibitor 1), BAGE-1 (B melanoma antigen-1), BCL-2 (B-cell CLL/lymphoma-2), BING-4 (WD repeat domain 46), CA 15-3/CA 27-29 (mucin 1), CA 19-9 (cancer antigen 19-9), CA 72-4 (cancer antigen 72-4), CA125 (cancer antigen 125), calreticulin, CAMEL (CTL-recognized antigen on melanoma), CASP-8 (caspase 8), cathepsin B, cathepsin L, CD19 (cluster of differentiation 19), CD20, CD22, CD25, CD30, CD33, CD4, CD52, CD55, CD56, CD80, CEA (carcinoembryonic antigen SG8), CLCA2 (chloride channel accessory 2), CML28 (chronic myelogenous leukemia tumor antigen 28), Coactosin-like protein, Collagen XXIII, COX-2 (cyclooxygenase-2), CT-9/BRD6 (cancer/testis antigen 9), Cten (c-terminal tensin-like protein), cyclin B1, cyclin D1, cyp-B, CYPB1 (cytochrome p450 family 1 subfamily b member 1), DAM-10/MAGE-B1 (melanoma-associated antigen B1), DAM-6/MAGE-B2, EGFR/Her1 (epidermal growth factor receptor), EMMPRIN (basigin), EpCam, EphA2 (EPH receptor A2), EphA3, ErbB3 (Erb-B2 receptor tyrosine kinase 3), EZH2 (enhancer of zeste 2 polycomb repressive complex 2 subunit), FGF-5 (fibroblast growth factor 5), FN (fibronectin), Fra-1 (Fosrelated antigen-1), G250/CAIX (carbonic anhydrase 9), GAGE-1 (G antigen-1), GAGE-2, GAGE-3, GAGE-4, GAGE-5, GAGE-6, GAGE-7b, GAGE-8, GDEP (gene differentially expressed in prostate), GnT-V (gluconate kinase), gp100 (melanocytes lineage-specific antigen GP100), GPC3 (glypican3), HAGE (helical antigen), HAST-2 (sulfotransferase family 1A member 1), hepsin, Her2/neu/ErbB2 (Erb-B2 receptor tyrosine kinase 2), HERV-K-MEL, HNE (medullasin), homeobox NKX 3.1, HOM-TES-14/SCP-1, HOM-TES-85, HPV-E6, HPVE7, HST-2 (sirtuin-2), hTERT, iCE (caspase 1), IGF-1R (insulin like growth factor-1 receptor), IL-13Ra2 (interleukin-13 receptor subunit α2), IL-2R (interleukin-2 receptor), IL-5 (interleukin-5), immature laminin receptor, kallikrein 2, kallikrein 4, Ki67, KIAA0205 (lysophosphatidylglycerol acyltransferase 1), KK-LC-1 (kita-kyushu lung cancer antigen-1), KM-HN-1, LAGE-1 (L antigen family member-1), Livin, MAGE-A1, MAGE-A10, MAGE-A12, MAGEA2, MAGE-A3, MAGE-A4, MAGE-A6, MAGE-A9, MAGE-B1, MAGE-B10, MAGE-B16, MAGEB17, MAGE-B2, MAGE-B3, MAGE-B4, MAGE-B5, MAGE-B6, MAGE-C1, MAGE-C2, MAGE-C3, MAGE-D1, MAGE-D2, MAGE-D4, MAGE-E1, MAGE-E2, MAGE-F1, MAGE-H1, MAGEL2 (melanoma antigen family L2), mammaglobin A, MART-1/Melan-A (melanoma antigen recognized by T-cells-1), MART-2, matrix protein 22, MC1R (melanocortin 1 receptor), M-CSF (macrophage colony-stimulating factor), Mesothelin, MG50/PXDN (peroxidasin), MMP 11 (matrix metalloprotease 11), MN/CA IX-antigen (carbonic anhydrase 9), MRP-3 (multidrug resistance-associated protein-3), MUC1 (mucin 1), MUC2, NA88-A (VENT-like homeobox 2 pseudogene 1), N-acetylglucos-aminyltransferase-V, Neo-PAP (Neo-poly (A) polymerase), NGEP (new gene expressed in prostate), NMP22 (nuclear matrix protein 22), NPM/ALK (nucleophosmin), NSE (neuron-specific enolase), NY-ESO-1, NY-ESO-B, OA1 (osteoarthritis QTL 1), OFA-iLRP (oncofetal antigen immature laminin receptor protein), OGT (O-GlcNAc transferase), OS-9 (endoplasmic reticulum lectin), osteocalcin, osteopontin, p15 (CDK inhibitor 2B), p53, PAGE-4 (P antigen family member-4), PAI-1 (plasminogen activator inhibitor-1), PAI-2, PAP (prostatic acid phosphatase), PART-1 (prostate androgen-regulated transcript 1), PATE (prostate and testis expressed 1), PDEF (prostate-derived Ets factor), Pim-1-Kinase (proviral integration site 1), Pin1 (Peptidyl-prolyl cis-trans isomerase NIMA-interacting 1), POTE (expressed in prostate, ovary, testis, and placenta), PRAME (preferentially expressed antigen in melanoma), prostein, proteinase-3, PSA (prostate-specific antigen), PSCA (prostate stem cell antigen), PSGR (prostate-specific G-protein coupled receptor), PSM, PSMA (prostate specific membrane antigen), RAGE-1 (renal tumor carcinoma antigen), RHAMM/CD168, RU1 (renal ubiquitous protein 1), RU2, SAGE (sarcoma antigen), SART-1 (squamous cell carcinoma antigen recognized by T-cells-1), SART-2, SART-3, Sp17 (sperm protein 17), SSX-1 (SSX family member 1), SSX-2/HOM-MEL-40, SSX-4, STAMP-1 (STEAP2 metalloreductase), STEAP, survivin, survivin-213, TA-90 (tumor associated antigen-90), TAG-72 (tumor associated glycoprotein-72), TARP (TCRγ alternate reading frame protein), TGFb (transforming growth factor β), TGFbR11 (transforming growth factor β receptor 11), TGM-4 (transglutaminase 4), TRAG-3 (taxol resistance associated gene 3), TRG (T-cell receptor γ locus), TRP-1 (transient receptor potential-1), TRP-2/6b, TRP-2/INT2, Trp-p8, Tyrosinase, UPA (U-plasminogen activator), VEGF (vascular endothelial growth factor A), VEGFR-2/FLK-1, and WT1 (wilms tumor 1).

34. The immune cell of claim 33, wherein the target antigen is CD19 or CD22.

35. The immune cell of claim 34, wherein the target antigen is CD19.

36. The immune cell of claim 33, wherein the target antigen is EGFR.

37. The immune cell of any of claims 33-36, wherein the target antigen is a cancer antigen whose expression is increased in or on the surface of a cancer cell, a cancer tissue, and/or a tumor microenvironment.

38. The immune cell of claim 32, wherein the target antigen is selected from the group consisting of:
α-actinin-4/m, ARTC1/m, bcr/abl, beta-Catenin/m, BRCA1/m, BRCA2/m, CASP-5/m, CASP-8/m, CDC27/m, CDK4/m, CDKN2A/m, CML66, COA-1/m, DEK-CAN, EFTUD2/m, ELF2/m, ETV6-AML1, FN1/m, GPNMB/m, HLA-A*0201-R170I, HLA-A11/m, HLA-A2/m, HSP70-2M, KIAA0205/m, K-Ras/m, LDLR-FUT, MART2/m, ME1/m, MUM-1/m, MUM-2/m, MUM-3/m, Myosin class 1/m, neo-PAP/m, NFYC/m, N-Ras/m, OGT/m, OS-9/m, p53/m, Pml/RARa, PRDX5/m, PTPRX/m, RBAF600/m, SIRT2/m, SYTSSX-1, SYT-SSX-2, TEL-AML1, TGFbRII, and TPI/m; and
wherein the target antigen is a cancer antigen that is a mutated form of antigen expressed in or on the surface of a cancer cell, a cancer tissue, and/or a tumor microenvironment.

39. The immune cell of any of claims 20-38, wherein expression of genes that weaken the function of the immune cell causes one or more of the following:
i) inhibition of proliferation of the immune cell;
ii) induction of cell death of the immune cell;
iii) inhibition of the ability of the immune cell to recognize the target antigen and/or undergo activation;
iv) induction of differentiation of the immune cell into a cell that does not induce immune response to the target antigen;
v) decreased reactios of the immune cell to a molecule which promotes immune response of the immune cell; or
vi) increased reaction of the immune cell to a molecule which suppresses immune response of the immune cell.

40. The immune cell of claim 39, wherein at least one or two of the genes that weaken the function of the immune cell arePD1, TGFBR1, or TGFBR2.

41. The immune cell of claim 39, wherein at least one or two of the genes that weaken the function of the immune cell increase reaction of the immune cell to a molecule which suppresses immune response of the immune cell.

42. The immune cell of claim 41, wherein at least one or two of the genes that increase reaction of the immune cell to a molecule which suppresses immune response of the immune cell encode an immune checkpoint receptor or ligand.

43. The immune cell of claim 42, wherein the immune checkpoint receptor is PD1.

44. The immune cell of any of claims 20-43, wherein the genetic disruption agent reduces the expression of at least two genes in the immune cell that weaken the function of the immune cell by at least 30, 40, 50, 60, 70, 80, 90, or 95% as compared to the immune cell in the absence of the genetic disruption agent.

45. The immune cell of claim 44, wherein the genetic disruption agent reduces the expression of at least two genes that increases reaction of the immune cell to a molecule which suppresses immune response of the immune cell.

46. The immune cell of claim 45, wherein the genetic disruption agent reduces the expression of at least two genes that encodes an immune checkpoint receptor or ligand.

47. The immune cell of claim 46, wherein the genetic disruption agent reduces the expression of at least one of: PD1, TGFBR1 TGFBR2

48. The immune cell of any of claims 44-47, wherein the genetic disruption agent reduces the expression of genes that weaken the function of the immune cell by RNA interference (RNAi).

49. The immune cell of claim 48, wherein the immune cell comprises more than one genetic disruption agent that reduce the expression of genes that weaken the function of the immune cell in the immune cell by RNAi.

50. The immune cell of claim 49, wherein different genetic disruption agents target different genes which weaken the function of the immune cell, for example, wherein a first genetic disruption agent targets a first gene and a second genetic disruption agent targets a second gene.

51. The immune cell of any of claims 48-50, wherein the RNAi is mediated by a short hairpin RNA (shRNA).

52. The immune cell of claim 51, wherein the RNAi is mediated by more than one shRNAs.

53. The immune cell of claim 52, wherein the RNAi is mediated by two shRNAs.

54. The immune cell of any of claims 52-53, wherein a first shRNA targets PD-1 and a second shRNA targets TGFBR1.

55. The immune cell of any of claims 52-53, wherein a first shRNA targets PD-1 and a second shRNA targets TGFBR2.

56. The immune cell of any one of claims 52-55, wherein the immune cell comprises nucleotide sequences that encode more than one shRNA.

57. The immune cell of claim 56, wherein the immune cell comprises nucleotide sequences that encode two shRNAs.

58. The immune cell of claim 57, wherein the nucleotide sequence encoding the two shRNAs comprises a nucleotide sequence selected from the group consisting of SEQ ID NOs.: 1-12 and SEQ ID NOs.: 13-23, respectively.

59. The immune cell of claim 57, wherein the two nucleotide sequence encoding the shRNAs comprises a nucleotide sequence selected from the group consisting of SEQ ID NOs.: 43-47 and SEQ ID NOs.: 13-23, respectively.

60. The immune cell of any of claims 56-59, wherein the nucleotide sequence encoding the shRNA(s) is present on a vector.

61. The immune cell of claim 60, wherein the expression of two different shRNAs is regulated by two different promoters.

62. The immune cell of claim 61, wherein the two different promoters are RNA polymerase III promoters.

63. The immune cell of claim 62, wherein the two promoters are U6 promoters.

64. The immune cell of claim 63, wherein the U6 promoters derived from different species.

65. The immune cell of any of claims 61-64, wherein the two promoters are oriented in different directions from each other.

66. The immune cell of any of claims 20-65, wherein the genetically engineered antigen receptor and the genetic disruption agent(s) are each expressed from a vector.

67. The immune cell of claim 66, wherein the genetically engineered antigen receptor and the genetic disruption agent(s) are expressed from the same vector.

68. The immune cell of any of claims 66-67, wherein the vector is a plasmid vector or a viral vector.

69. The immune cell of claim 68, wherein the viral vector is a lentivirus vector, adenovirus vector or adeno-associated viral vector.

70. The immune cell of claim 69, wherein the lentivirus vector is a retrovirus vector.

71. The immune cell of any of claims 20-70, wherein the immune cell is selected from the group consisting of a T cell and a natural killer (NK) cell.

72. The immune cell of claim 71, wherein the immune cell is a T cell.

73. The immune cell of claim 72, wherein the T cell is a CD4+ T cell or a CD8+ T cell.

74. The immune cell of any of claim 72 or 73, wherein the immune cell comprises nucleotide sequences that encode two shRNAs and a CAR or mTCR on the same vector.

75. The immune cell of claim 74, wherein the two shRNA are each regulated by two different RNA polymerase III promoters oriented in different directions from each other.

76. The immune cell of claim 75, wherein the CAR targets CD19, the first shRNA targets PD-1, and the second shRNA targets TGFBR1.

77. The immune cell of claim 75, wherein the CAR targets CD19, the first shRNA targets PD-1, and the second shRNA targets TGFBR2.

78. The immune cell of claim 75, wherein the CAR targets CD19, the first shRNA targets PD-1, and the second shRNA targets TGFBR2.

79. A method of producing an immune cell comprising introducing into an immune cell, simultaneously or sequentially in any order:
(1) a gene encoding a genetically engineered antigen receptor that specifically binds to a target antigen; and
(2) a genetic disruption agent, wherein the genetic disruption agent, or expression thereof, reduces or is capable of reducing expression in the immune cell of at least two genes that weaken the function of the immune cell,
thereby producing an immune cell in which a genetically engineered antigen receptor is expressed and expression of two genes that weaken the function of the immune cell is reduced.

80. The method of claim 79, wherein the genetically engineered antigen receptor is a chimeric antigen receptor (CAR) or a T cell receptor (TCR).

81. The method of claim 80, wherein the genetically engineered antigen receptor is a CAR.

82. The method of claim 81, wherein the CAR comprises an extracellular antigen recognition domain, a transmembrane domain, and an intracellular signal transduction domain.

83. The method of claim 82, wherein the extracellular antigen recognition domain of the CAR specifically binds to the target antigen.

84. The method of claim 83, wherein the target antigen is EGFR.

85. The method of claim 82, wherein the intracellular signal transduction domain of the CAR comprises an intracellular domain of a CD3 zeta (CD3ζ) chain.

86. The method of claim 85, wherein the intracellular signal transduction domain of the CAR further comprises a costimulatory molecule.

87. The method of claim 86, wherein the costimulatory molecule is selected from the group consisting of ICOS, 0X40, CD137 (4-1BB), CD27, and CD28.

88. The method of claim 87, wherein the costimulatory molecule is CD137 (4-1BB).

89. The method of claim 87, wherein the costimulatory molecule is CD28.

90. The method of claim 80, wherein the genetically engineered antigen receptor is a TCR.

91. The method of claim 90, wherein the TCR is a monoclonal TCR (mTCR).

92. The method of any of claims 79-91, wherein the target antigen is expressed in or on the surface of a cancer cell, a cancer tissue, and/or a tumor microenvironment.

93. The method of claim 92, wherein the target antigen is selected from the group consisting of:
5T4 (Trophoblast glycoprotein), 707-AP, 9D7, AFP (a-fetoprotein), AlbZIP (androgen-induced bZIP), HPG1 (human prostate specific gene-1), α5β1-Integrin, α5β6-Integrin, α-methylacyl-coenzyme A racemase, ART-4 (ADPribosyltransferase-4), B7H4 (v-set domain-containing T-cell activation inhibitor 1), BAGE-1 (B melanoma antigen-1), BCL-2 (B-cell CLL/lymphoma-2), BING-4 (WD repeat domain 46), CA 15-3/CA 27-29 (mucin 1), CA 19-9 (cancer antigen 19-9), CA 72-4 (cancer antigen 72-4), CA125 (cancer antigen 125), calreticulin, CAMEL (CTL-recognized antigen on melanoma), CASP-8 (caspase 8), cathepsin B, cathepsin L, CD19 (cluster of differentiation 19), CD20, CD22, CD25, CD30, CD33, CD4, CD52, CD55, CD56, CD80, CEA (carcinoembryonic antigen SG8), CLCA2 (chloride channel accessory 2), CML28 (chronic myelogenous leukemia tumor antigen 28), Coactosin-like protein, Collagen XXIII, COX-2 (cyclooxygenase-2), CT-9/BRD6 (cancer/testis antigen 9), Cten (c-terminal tensin-like protein), cyclin B1, cyclin D1, cyp-B, CYPB1 (cytochrome p450 family 1 subfamily b member 1), DAM-10/MAGE-B1 (melanoma-associated antigen B1), DAM-6/MAGE-B2, EGFR/Her1 (epidermal growth factor receptor), EMMPRIN (basigin), EpCam, EphA2 (EPH receptor A2), EphA3, ErbB3 (Erb-B2 receptor tyrosine kinase 3), EZH2 (enhancer of zeste 2 polycomb repressive complex 2 subunit), FGF-5 (fibroblast growth factor 5), FN (fibronectin), Fra-1 (Fosrelated antigen-1), G250/CAIX (carbonic anhydrase 9), GAGE-1 (G antigen-1), GAGE-2, GAGE-3, GAGE-4, GAGE-5, GAGE-6, GAGE-7b, GAGE-8, GDEP (gene differentially expressed in prostate), GnT-V (gluconate kinase), gp100 (melanocytes lineage-specific antigen GP100), GPC3 (glypican3), HAGE (helical antigen), HAST-2 (sulfotransferase family 1A member 1), hepsin, Her2/neu/ErbB2 (Erb-B2 receptor tyrosine kinase 2), HERV-K-MEL, HNE (medullasin), homeobox NKX 3.1, HOM-TES-14/SCP-1, HOM-TES-85, HPV-E6, HPVE7, HST-2 (sirtuin-2), hTERT, iCE (caspase 1), IGF-1R (insulin like growth factor-1 receptor), IL-13Ra2 (interleukin-13 receptor subunit α2), IL-2R (interleukin-2 receptor), IL-5 (interleukin-5), immature laminin receptor, kallikrein 2, kallikrein 4, Ki67, KIAA0205 (lysophosphatidylglycerol acyltransferase 1), KK-LC-1 (kita-kyushu lung cancer antigen-1), KM-HN-1, LAGE-1 (L antigen family member-1), Livin, MAGE-A1, MAGE-A10, MAGE-A12, MAGEA2, MAGE-A3, MAGE-A4, MAGE-A6, MAGE-A9, MAGE-B1, MAGE-B10, MAGE-B16, MAGEB17, MAGE-B2, MAGE-B3, MAGE-B4, MAGE-B5, MAGE-B6, MAGE-C1, MAGE-C2, MAGE-C3, MAGE-D1, MAGE-D2, MAGE-D4, MAGE-E1, MAGE-E2, MAGE-F1, MAGE-H1, MAGEL2 (melanoma antigen family L2), mammaglobin A, MART-1/Melan-A (melanoma antigen recognized by T-cells-1), MART-2, matrix protein 22, MC1R (melanocortin 1 receptor), M-CSF (macrophage colony-stimulating factor), Mesothelin, MG50/PXDN (peroxidasin), MMP 11 (matrix metalloprotease 11), MN/CA IX-antigen (carbonic anhydrase 9), MRP-3 (multidrug resistance-associated protein-3), MUC1 (mucin 1), MUC2, NA88-A (VENT-like homeobox 2 pseudogene 1), N-acetylglucos-aminyltransferase-V, Neo-PAP (Neo-poly (A) polymerase), NGEP (new gene expressed in prostate), NMP22 (nuclear matrix protein 22), NPM/ALK (nucleophosmin), NSE (neuron-specific enolase), NY-ESO-1, NY-ESO-B, OA1 (osteoarthritis QTL 1), OFA-iLRP (oncofetal antigen immature laminin receptor protein), OGT (O-GlcNAc transferase), OS-9 (endoplasmic reticulum lectin), osteocalcin, osteopontin, p15 (CDK inhibitor 2B), p53, PAGE-4 (P antigen family member-4), PAI-1 (plasminogen activator inhibitor-1), PAI-2, PAP (prostatic acid phosphatase), PART-1 (prostate androgen-regulated transcript 1), PATE (prostate and testis expressed 1), PDEF (prostate-derived Ets factor), Pim-1-Kinase (proviral integration site 1), Pin1 (Peptidyl-prolyl cis-trans isomerase NIMA-interacting 1), POTE (expressed in prostate, ovary, testis, and placenta), PRAME (preferentially expressed antigen in melanoma), prostein, proteinase-3, PSA (prostate-specific antigen), PSCA (prostate stem cell antigen), PSGR (prostate-specific G-protein coupled receptor), PSM, PSMA (prostate specific membrane antigen), RAGE-1 (renal tumor carcinoma antigen), RHAMM/CD168, RU1 (renal ubiquitous protein 1), RU2, SAGE (sarcoma antigen), SART-1 (squamous cell carcinoma antigen recognized by T-cells-1), SART-2, SART-3, Sp17 (sperm protein 17), SSX-1 (SSX family member 1), SSX-2/HOM-MEL-40, SSX-4, STAMP-1 (STEAP2 metalloreductase), STEAP, survivin, survivin-213, TA-90 (tumor associated antigen-90), TAG-72 (tumor associated glycoprotein-72), TARP (TCRγ alternate reading frame protein), TGFb (transforming growth factor β), TGFbR11 (transforming growth factor β receptor 11), TGM-4(transglutaminase 4), TRAG-3 (taxol resistance associated gene 3), TRG (T-cell receptor γ locus), TRP-1 (transient receptor potential-1), TRP-2/6b, TRP-2/INT2, Trp-p8, Tyrosinase, UPA (U-plasminogen activator), VEGF (vascular endothelial growth factor A), VEGFR-2/FLK-1, and WT1 (wilms tumor 1).

94. The method of claim 93, wherein the target antigen is CD19 or CD22.

95. The method of claim 94, wherein the target antigen is CD19.

96. The method of claim 93, wherein the target antigen is EGFR.

97. The method of any of claims 93-96, wherein the target antigen is a cancer antigen, wherein the cancer antigen is an antigen whose expression is increased in or on the surface of a cancer cell, a cancer tissue, and/or a tumor microenvironment.

98. The method of claim 92, wherein the target antigen is selected from the group consisting of:
α-actinin-4/m, ARTC1/m, bcr/abl, beta-Catenin/m, BRCA1/m, BRCA2/m, CASP-5/m, CASP-8/m, CDC27/m, CDK4/m, CDKN2A/m, CML66, COA-1/m, DEK-CAN, EFTUD2/m, ELF2/m, ETV6-AML1, FN1/m, GPNMB/m, HLA-A*0201-R170I, HLA-A11/m, HLA-A2/m, HSP70-2M, KIAA0205/m, K-Ras/m, LDLR-FUT, MART2/m, ME1/m, MUM-1/m, MUM-2/m, MUM-3/m, Myosin class 1/m, neo-PAP/m, NFYC/m, N-Ras/m, OGT/m, OS-9/m, p53/m, Pml/RARa, PRDX5/m, PTPRX/m, RBAF600/m, SIRT2/m, SYTSSX-1, SYT-SSX-2, TEL-AML1, TGFbRII, and TPI/m; and
wherein the target antigen is a cancer antigen, wherein the cancer antigen is a mutated form of antigen expressed in or on the surface of a cancer cell, a cancer tissue, and/or a tumor microenvironment.

99. The method of any of claims 79-98, wherein expression of genes that weaken the function of the immune cell causes one or more of the following:
i) inhibition of proliferation of the immune cell;
ii) induction of cell death of the immune cell;
iii) inhibition of the ability of the immune cell to recognize the target antigen and/or to get activated;
iv) induction of differentiation of the immune cell into a cell that does not induce immune response to the target antigen;
v) decreased reaction of the immune cell to a molecule which promotes immune response of the immune cell; or
vi) increased reaction of the immune cell to a molecule which suppresses immune response of the immune cell.

100. The method of claim 99, wherein at least one or two of the genes that weaken the function of the immune cell are PD1, TGFBR1, or TGFBR2.

101. The method of claim 99, wherein at least one or two of the genes that weaken the function of the immune cell increase reaction of the immune cell to a molecule which suppresses immune response of the immune cell.

102. The method of claim 101, wherein at least two genes that increase reaction of the immune cell to a molecule which suppresses immune response of the immune cell encode an immune checkpoint receptor and ligand.

103. The method of claim 102, wherein the immune checkpoint receptor ligand is PD1.

104. The method of any of claims 79-103, wherein the genetic disruption agent reduces the expression of at least two genes in the immune cell that weaken the function of the immune cell by at least 30, 40, 50, 60, 70, 80, 90, or 95% as compared to the immune cell in the absence of the genetic disruption agent(s).

105. The method of claim 104, wherein the genetic disruption agent reduces the expression of at least two genes that increase reaction of the immune cell to a molecule which suppresses immune response of the immune cell.

106. The method of claim 105, wherein the genetic disruption agent reduces the expression of at least two genes that encode an immune checkpoint receptor or ligand.

107. The method of claim 106, wherein the genetic disruption agent reduces the expression of PD1 and TGFBR1.

108. The method of claim 110, wherein the genetic disruption agent reduces the expression of PD1 and TGFBR2.

109. The method of any of claims 105-108, wherein the genetic disruption agent reduces the expression of at least two genes that weaken the function of the immune cell by RNA interference (RNAi).

110. The method of claim 109, wherein more than one genetic disruption agents reduce the expression of genes that weaken the function of the immune cell in the immune cell by RNAi.

111. The method of claim 110, wherein the genetic disruption agents target different genes which weaken the function of the immune cell wherein a first genetic disruption agent targets a first gene and a second genetic disruption agent targets a second gene.

112. The method of any of claims 109-111, wherein the RNAi is mediated by a short hairpin RNA (shRNA).

113. The method of claim 112, wherein the RNAi is mediated by more than one shRNA.

114. The method of claim 113, wherein the RNAi is mediated by two shRNAs.

115. The method of claim 113 or 114, wherein a first shRNA targets PD-1 and a second shRNA targets TGFBR1.

116. The method of claim 113 or 114, wherein a first shRNA targets PD-1 and a second shRNA targets TGFBR2.

117. The method of any of claims 114-116, wherein the immune cell comprises nucleotide sequences that encode a shRNA.

118. The method of claim 117, wherein the immune cell comprises nucleotide sequences that encode more than one shRNA.

119. The method of claim 117, wherein the immune cell comprises nucleotide sequences that encode two shRNAs.

120. The method of any of claims 117-119, wherein the two nucleotide sequences encoding the shRNAs comprise sequences selected from the groups consisting of SEQ ID NOs.: 1-12 and SEQ ID NOs.: 13-23, respectively.

121. The method of any of claims 117-119, wherein the two nucleotide sequences encoding the shRNAs comprise sequences selected from the groups consisting of SEQ ID NOs.: 43-47 and SEQ ID NOs.: 13-23, respectively.

122. The method of any of claims 117-119, wherein the nucleotide sequences encoding the shRNA is present on a vector.

123. The method of claim 122, wherein the expression of different shRNAs is respectively regulated by different promoters.

124. The method of claim 123, wherein the expression of two different shRNAs is respectively regulated by two different promoters.

125. The method of claim 124, wherein the two different promoters are RNA polymerase III promoters.

126. The method of claim 125, wherein the two promoters are U6 promoters.

127. The method of claim 126, wherein the U6 promoters derive from different species.

128. The method of any of claims 124-127, wherein the two promoters are oriented in different directions from each other.

129. The method of any of claims 79-128, wherein the genetically engineered antigen receptor and the genetic disruption agent(s) are each expressed from a vector.

130. The method of claim 129, wherein the genetically engineered antigen receptor and the genetic disruption agent(s) are expressed from the same vector.

131. The method of any of claim 129 or 130, wherein the vector is a plasmid vector or a viral vector.

132. The method of claim 131, wherein the viral vector is a lentivirus vector, adenovirus vector or adeno-associated viral vector.

133. The method of claim 132, wherein the lentivirus vector is a retrovirus vector.

134. The method of any of claims 79-133, wherein the immune cell is selected from the group consisting of a T cell and a natural killer (NK) cell.

135. The method of claim 134, wherein the immune cell is a T cell.

136. The method of claim 135, wherein the T cell is a CD4+ T cell or a CD8+ T cell.

137. The method of claim 135 or 136, wherein the immune cell comprises nucleotide sequences that encode two shRNAs and a CAR on the same vector.

138. The method of claim 137, wherein the two shRNAs are each regulated by two different RNA polymerase III promoters oriented in different directions from each other.

139. The method of claim 138, wherein the CAR targets CD19, the first shRNA targets PD-1, and the second shRNA targets TGFBR1.

140. The method of claim 138, wherein the CAR targets CD19, the first shRNA targets PD-1, and the second shRNA targets TGFBR2.

141. The method of claim 138, wherein the CAR targets EGFR, the first shRNA targets PD-1, and the second shRNA targets TGFBR2

142. A composition comprising the immune cell of any of claims 20-78.

143. A pharmaceutical composition comprising the immune cell of any of claims 20-78 and a pharmaceutically acceptable carrier.

144. A method of treatment comprising administering to a subject having a disease or condition in need of immune therapy the immune cell of any of claims 20-78 or the composition of claim 142 or 143.

145. The method of claim 144, wherein the genetically engineered antigen receptor specifically binds to an antigen associated with the disease or the condition.

146. The method of claim 144 or 145, wherein the disease or the condition is a cancer, e.g., a tumor.

147. The immune cell of any of claims 20-78 or the composition of claim 142 or 143 for use in treating a disease or a condition.

148. Use of the immune cell of any of claims 21-82 or the composition of claim 125 or 126 in the manufacture of a medicament for treating a disease or a condition.

149. The immune cells or the composition of claim 147 or the use of claim 148, wherein the genetically engineered antigen receptor specifically binds to an antigen associated with the disease or the condition.

150. The use, composition, or immune cell of claim 148 or claim 149, wherein the disease or the condition is a cancer, e.g. a tumor.

151. The use, composition, or immune cell of claim 150, wherein the cancer is non-Hodgkin's lymphoma.

152. The vector according to claim 6, wherein the genes weakening the function of immune cells are PD-1 and TGFBR1.

153. The vector according to claim 6, wherein the genes weakening the function of immune cells are PD-1 and TGFBR2.

154. The immune cell of claim 40, wherein the genes that weaken the function of the immune cell are PD1 and TGFBR1.

155. The immune cell of claim 40, wherein the genes that weaken the function of the immune cell are PD1 and TGFBR2.

156. The immune cell of claim 47, wherein the genetic disruption agent reduces the expression of PD1 and TGFBR1.

157. The immune cell of claim 47, wherein the genetic disruption agent reduces the expression of PD1 and TGFBR2.

158. The method of claim 100, wherein the genes that weaken the function of the immune cell are PD1, and TGFBR1.

159. The method of claim 100, wherein the genes that weaken the function of the immune cell are PD1, and TGFBR2.
